(19) United States
(12) Patent Application Publication
(10) Pub. No.: US 2025/0345605 A1
(43) Pub. Date: Nov. 13, 2025

(54) DEVICES, SYSTEMS AND METHODS FOR ENHANCING SLEEP

(71) Applicant: Electrocore, Inc., Rockaway, NJ (US)

(72) Inventors: Bruce J. Simon, Santa Fe, NM (US); Joseph P. Errico, Palm Beach Gardens, FL (US)

(21) Appl. No.: 19/258,809

(22) Filed: Jul. 2, 2025

Related U.S. Application Data
(63) Continuation-in-part of application No. 18/754,919, filed on Jun. 26, 2024.
Application No. 18/754,919 is a continuation of application No. 17/318,824, filed on May 12, 2021, now Pat. No. 12,053,631.
Application No. 17/318,824 is a continuation of application No. 16/511,953, filed on Jul. 15, 2019, now Pat. No. 11,027,127.
Application No. 16/511,953 is a continuation of application No. 15/232,158, filed on Aug. 9, 2016, now Pat. No. 10,350,411.
Application No. 15/232,158 is a division of application No. 14/212,992, filed on Mar. 14, 2014, now Pat. No. 9,427,581.
Application No. 14/212,992 is a continuation-in-part of application No. 13/872,116, filed on Apr. 28, 2013, now Pat. No. 9,254,383.

(51) Int. Cl. A61N 1/36; A61B 5/00; A61B 5/024; A61B 5/394; A61B 90/00; A61N 1/04; A61N 1/40

(52) U.S. Cl. CPC A61N 1/36021; A61B 5/394; A61B 5/4035; A61B 5/4041; A61B 5/4803; A61B 5/4836; A61B 5/7264; A61B 5/7267; A61N 1/0456; A61N 1/36031; A61N 1/36034; A61B 5/02405; A61B 2090/3941; A61N 1/40

(57) ABSTRACT

Systems, methods and devices are provided for delivering a combination of electrical impulses (and/or fields) and various stimuli to bodily tissues for various purposes. The combined therapy increases the brain's capacity to clear metabolic and neurotoxic material (waste removal), facilitates neuroplasticity to affect brain network optimization (learning and memory consolidation), and restores brain energy and neurotransmitter levels (neurometabolic restoration). In certain aspects, the systems and methods are particularly useful for enhancing sleep quantity, quality, and/or efficiency to promote more effective sleep, including more efficient waste clearance, memory consolidation, neurotransmitter rebalancing, and maintenance of energy homeostasis, leading to enhanced neurological health and reduced required sleep duration. The systems and methods may also be useful for improving a cognitive performance of the user by maintaining cognitive function during sleep deprivation and/or temporarily or permanently improving intelligence, learning capacity, memory retention, recall, mood and/or alertness.

### CROSS REFERENCE TO RELATED APPLICATIONS

This application is a Continuation in Part of U.S. application Ser. No. 18/754,919, filed Jun. 26, 2024, which is a Continuation of U.S. application Ser. No. 17/318,824, filed May 12, 2021, now U.S. Pat. No. 12,053,631 issued Aug. 6, 2024; which is a Continuation of U.S. application Ser. No. 16/511,953, filed Jul. 15, 2019, now U.S. Pat. No. 11,027,127 issued Jun. 8, 2021; which is a Continuation of U.S. application Ser. No. 15/232,158 filed Aug. 9, 2016, now U.S. Pat. No. 10,350,411 issued Jul. 16, 2019; which is a Divisional of U.S. application Ser. No. 14/212,992 filed Mar. 14, 2014, now U.S. Pat. No. 9,427,581 issued Aug. 30, 2016; the complete disclosures of which are incorporated herein by reference for all purposes.

### FIELD

This description generally relates to devices, systems and methods for enhancing sleep quantity, quality, and/or efficiency and more particularly to systems and methods for combining various audio, visual and/or other stimuli with nerve stimulation to promote more effective sleep, including reduced neuroinflammation, more efficient waste clearance, memory consolidation, neurotransmitter rebalancing, and maintenance of energy homeostasis, leading to enhanced neurological health and reduced required sleep duration.

### BACKGROUND

Sleep serves three critical purposes: (i) clearance of metabolic and neurotoxic material (waste removal), (ii) facilitation of neuroplasticity to affect brain network optimization (learning and memory consolidation), and (iii) restoration of brain energy and neurotransmitter levels (neurometabolic restoration). Each of these functions contributes to the capacity of the brain to perform cognitively, emotionally, and physiologically.

Research has shown that sleep restriction leads to impaired immune, metabolic, and cognitive functions, and may well even result in disruptions of the gut microbiome. With reference to the graph provided in FIG. 16, taken from Belenky, Gregory, Nancy J. Wesensten, David R. Thorne, Maria L. Thomas, Helen C. Sing, Daniel P. Redmond, Michael B. Russo, and Thomas J. Balkin. “Patterns of performance degradation and restoration during sleep restriction and subsequent recovery: A sleep dose-response study.” Journal of sleep research 12, no. 1 (2003): 1-12., which is hereby incorporated by reference), and specifically with respect to loss of cognitive performance (as measured by speed of response during a psychomotor vigilance test (PVT)), restriction to five hours of sleep per night for seven nights leads to a reduction in performance of 10-20% within a period of approximately five days, after which, performance plateaus at this reduced level. Curiously, the opportunity for recovery sleep does not lead to an immediate restoration of full function, i.e., return to pre-restriction performance, even after three days. This observation suggests that repeated sleep restriction leads to a state shift in the brain that is affected by sleep loss, but is not improved by restoration of sleep alone. This phenomenon is reminiscent of the priming of the immune cells of the brain that occurs in animal models of pain sensitization see FIG. 17, wherein repeated administrations of inflammatory media to the dura of the brain leads to a permanent pain state and enhanced responsiveness to pain-triggering stimuli, even after the inflammatory media has cleared (reference Oshinsky, Michael L., Angela L. Murphy, Hugh Hekierski Jr, Marnie Cooper, and Bruce J. Simon. “Noninvasive vagus nerve stimulation as treatment for trigeminal allodynia.” Pain 155, no. 5 (2014): 1037-1042., which is hereby incorporated by reference). This permanent pain state is associated with elevated microglial activation (i.e., inflammation) and a dysregulation in neurotransmitter synthesis, neurotransmitter receptor populations, and oxidative stress.

Referring again to the graph provided in FIG. 16, extreme sleep restriction (e.g., three hours per night for seven nights) results in an even more significant onset of performance deficit that exhibits a severe progressive degradation in performance that does not plateau. Curiously, recovery sleep, after severe restriction, does restore a portion of deficit incurred, with function rebounding partially within a single night of recovery. However, this restoration only restores cognitive performance to a level comparable to a reduced plateau of 20% deficit, similar to that experienced by the five hour per night cohort.

The two defining differences between the three and five hour per day restriction of sleep, i.e., (i) the loss of performance plateauing versus a progressive decline in function; and (ii) the partial versus no restoration of function during a three-day recovery sleep period, reflect two separate mechanisms. The first, (i.e., the progressive loss of function) is explained by the fact that three hours of sleep is insufficient to clear neurotoxic waste from the brain. As a result, the build-up of waste leads to an unrelenting and progressive deficit. This incomplete glymphatic clearance (which, importantly, also becomes impaired by inflammatory processes discussed more fully hereinbelow) threatens the viability of the brain with ever-increasing severity. Permanent brain damage and even death can result from this escalating neurotoxicity. Important neurotoxic compounds that must be cleared include amyloid protein, which is found in elevated concentrations in individuals with progressive sleep deficits and may explain the correlation between a history of such sleep restriction and neurodegenerative conditions. Five hours of sleep per night, however, appears to permit sufficient clearance of neurotoxic waste that the brain can cope with (sometimes by slowing down the production of additional waste by impairing metabolic function and/or the removal of overly-active synaptic connections—an observed phenomenon that occurs in chronically sleep restricted individuals—a discussion of both is also provided hereinbelow).

With respect to the restoration of function during a short period (e.g., 3-days) of recovery sleep, the more extremely sleep restricted individuals experience partial recovery. This is likely the result of prolonged waste clearance via glymphatic flow (even if still impaired) which dominates the sleep architecture during these nights. That is, the proportion of sleep cycles dedicated to glymphatic clearance is atypically longer, leading to a deficit in REM sleep in the nights following chronic restriction. It is important to note that the recovery can only be to the level experienced by less severely sleep restricted, which is incomplete. This is believed to be the result of a priming of the immune cells of the brain (i.e., microglia) through repeated inflammatory insults associated with the sleep restriction period. These insults are attributable to incomplete or missing network optimization and neurotransmitter/receptor balancing functions required to support optimized cognitive function, much of which appears to be semi-permanently altered by prolonged sleep restriction, even of only a couple of hours per night.

Current use of hypnotics and stimulants to force sleep and to temporarily improve daytime alertness have considerable drawbacks. Hypnotics often impair the user's capacity to attain a necessary level of alertness while under the influence, which can be life-threatening under potential critical conditions (i.e., medical, military, and disaster response conditions, which are all conditions associated with chronic sleep restriction). Prolonged use of same can lead to dependency for achieving the sleep state, leading to risks of future insomnia. Most importantly, under severe sleep restriction, when neurotoxic waste clearance is impaired in time and efficiency by inflammation, hypnotics often contribute to this impairment, thereby reducing the effectiveness of the recovery of sleep. Similarly, hypnotics impair the network optimization that normally occurs during sleep (slow wave sleep, SWS, and REM), and therefore, lead to failures to consolidate memories and learn efficiently.

Similarly, the stimulants that are currently employed to enhance alertness simply mask the underlying cognitive challenges associated with reduced sleep, and do not enhance neurotoxic waste clearance or network optimization. That is, memory and learning deficiencies are not improved while using stimulants. Dependencies can also result from prolonged use, with dangerous withdrawal symptoms and associated erratic emotional behavior.

### SUMMARY

Systems, devices and methods are provided for delivering a combination of electrical impulses (and/or fields) and various stimuli to a user for various purposes. In certain aspects, the systems, devices and methods are particularly useful for enhancing sleep quantity, quality, and/or efficiency to promote faster sleep onset, deeper sleep stages and enhanced neurological health and/or to reduce the sleep duration required to maintain cognitive performance and overall neurological health. The systems, devices and methods may also be useful for improving a cognitive performance of the user by maintaining cognitive function during sleep deprivation and/or temporarily or permanently improving intelligence, learning capacity, memory retention, recall, mood and/or alertness. More particularly, these systems, devices, and methods enhance glymphatic clearance, maintain REM-like sleep periods, and regulate sleep micro-architecture to enhance cognitive performance despite sleep restriction.

In one aspect, a system for improving sleep comprises a nerve stimulator comprising an electrode configured for contacting the outer skin surface at, or near, a target location and an energy source coupled to the stimulator. The energy source is configured to generate at least one electrical impulse and to transmit the at least one electrical impulse transcutaneously from the electrode through the outer skin surface of the user to a selected nerve in the user adjacent to, or near, the target location. The system further comprises a sensory stimulator configured to deliver one or more stimuli to the sense organs or brain of a user. The sensory stimulator may comprise one or more of a visual stimulator, an auditory stimulator, an olfactory stimulator, a tactile stimulator or a combination thereof.

The combined therapy described herein increases the brain's capacity to clear metabolic and neurotoxic material (waste removal), facilitates neuroplasticity to affect brain network optimization (learning and memory consolidation), and restores brain energy and neurotransmitter levels (neurometabolic restoration). Applicant has discovered that the mechanisms underlying each of these functions of sleep can be optimized, and that the consequences of operating under extreme sleep restriction can be minimized by addressing them with a combined approach involving both neuroimmune and targeted neural entrainment approaches. More specifically, Applicants have discovered that the progressive degradation in cognitive performance experienced by individuals with sleep deprivation can be slowed or halted with the combined therapy described herein.

The nerve stimulation may be delivered to the user prior to sleep, during sleep or after the user has wakened. The sensory stimulation may be delivered to the user prior to sleep, during sleep or after the user has wakened. In an exemplary embodiment, the nerve stimulation is delivered prior to sleeping and the sensory stimulation is delivered during sleeping. The nerve stimulation functions as a brain “preconditioning” that reduces reorients microglia and astrocytes into a non-inflammatory posture to increase the effectiveness of the sensory stimulation during sleep. Specifically, the nerve stimulation reduces inflammatory signaling and facilitates efficient glymphatic flow and changes to macro- and micro-sleep architecture to facilitate memory consolidation processes. The nerve and stimuli stimulations together reinforce slow wave sleep (SWS) while providing neurotoxin-clearing gamma stimulation through the carrier tone, and restore benefits normally associated with REM sleep.

In embodiments, the sensory stimulator comprises an auditory stimulator. The auditory stimuli may include, but is not limited to, white noise, lower frequency alternatives to white noise, such as pink/brown noise, red noise, nature sounds, binaural beats, music therapy and the like.

In embodiments, the sensory stimulator comprises a visual stimulator. The visual stimuli may include, but is not limited to, light patterns, flashing patterns of specific frequencies (40 Hz pulses of 650 nm red light being of particular value), warm light, dimming lights, light therapy, sunset stimulation and the like.

In embodiments, the sensory stimulator comprises an olfactory stimulator. The olfactory stimuli may include, but is not limited to, lavender, chamomile, cedarwood, sandalwood, Ylang Ylang, essential oil diffusers and the like.

In embodiments, the sensory stimulator comprises a tactile stimulator. The tactile stimuli may include, but is not limited to, low-level vibrations (including very low frequency diffuse ultra sound), temperature stimuli (e.g., cooling mattresses or heated blankets) and/or deep pressure stimulation.

In embodiments, the sensory stimulator comprises both an auditory stimulator and a visual stimulator. In an exemplary embodiment, the auditory stimulator is configured to deliver binaural tones or beats to the brain of the user. Binaural beats (BNB) are defined as an auditory illusion created when two tones with different frequencies are delivered separately into each ear of the user. The brain perceives a third tone or a “binaural beat” which is the difference between the two frequencies.

In embodiments, the binaural tones have a carrier frequency of about 10 Hz to about 100 Hz, or about 20 Hz to about 80 Hz, or about 30 Hz to about 50 Hz, or about 40 Hz. The binaural tones have a beat frequency of about 0.1 Hz to about 10 Hz, or about 0.2 Hz to about 2 Hz, or about 0.5 Hz to about 1.5 Hz, or about 1 Hz. An example of carrier frequencies for binaural beats of 1 Hz might be a lef channel of 40 Hz and a right channel of 41 Hz, each delivered o a respective ear. Lower frequencies (e.g., 0.5 −4 Hz delta waves) promote detailed memory consolidation and glymphatic clearance, characteristic of deep sleep (NREM N3), while higher frequencies (e.g., 25 to 50 Hz gamma waves) facilitate procedural memory consolidation and emotional processing, typically associated with REM sleep.

In embodiments, the visual stimulator is configured to deliver one or more light patterns to the user. In an exemplary embodiment, the light patterns are synchronized with the binaural tones. By delivering synchronized binaural tones and red light at these distinct frequencies (e.g., 0.5 Hz delta beat with soothing carrier, including 40 Hz gamma carrier if, or 40 Hz delta carrier), the system induces concurrent brainwave activities, enabling restorative and cognitive benefits within a single sleep session.

In certain embodiments, the light patterns may comprise waves at a wavelength of 580 nm to 830 nm, or about 630 nm to about 730 nm, or about 650 nm. The patterns may be flickering pulses lasting 1 ms to about 10 ms, to about 2 ms to 5 ms, or about 2.5 ms, repeated at about 20 Hz to about 80 Hz, or about 30 Hz to about 50 Hz, or about 40 Hz.

In embodiments, the light waves may have a wavelength of about 380 nm to about 750 nm (i.e., visible light). In an exemplary embodiment, the wavelength may fall within a range of one or more specific colors, such as blue, red, green or the like. In one such embodiment, the wavelength falls in, or near, the red wavelength, or about 600 nm to about 750 nm, or about 610 nm to about 680 nm or about 620 nm to about 650 nm. The red light also causes the dissociation of nitric oxide from cytochrome c oxidase in mitochondria, supporting cellular metabolic efficiency, and also minimizes disruption of melatonin production in the body and provides circadian rhythm support.

In embodiments, the system further comprises one or more sensors coupled to the stimuli stimulator. The sensors are configured to detect one or more physiological parameters of the user. In an exemplary embodiment, the sensors comprise EEG sensors configured to measure voltage differences between pairs of electrodes positioned on a scalp of the user. These voltages may, for example, reflect summed electrical activity of neurons from the brain of the user. This summed electrical activity may represent differential electrical activity or the difference in voltage between two locations, which may, for example, represent a differential brainwave activity.

In embodiments, the system further comprises a computer readable media comprising non-transitory computer executable instructions which, when executed by at least one electronic processor, computes an effective brainwave frequency based on the differential brainwave activity detected by the sensors. In an exemplary embodiment, the computer readable media comprises non-transitory computer executable instructions which, when executed by at least one electronic processor synchronizes the binaural tones with the effective brainwave frequency.

In embodiments, the nerve stimulator comprises at least one electrode configured for contact with the user's skin on, or near, the target nerve. In an exemplary embodiment, the target nerve is the vagus nerve. The electrical impulses delivered by the electrode are sufficient to reorient microglia and astrocytes into a non-inflammatory posture prior to sleep.

In certain embodiments, the electrical impulse comprises pulses having a frequency of about 1 kHz to about 20 kHz. The electrical impulse may comprise bursts of pulses, with each burst having a frequency of about 1 to about 100 bursts per second and each pulse has a duration of about 50 to about 1000 microseconds in duration. The bursts each comprise about 2 to 20 pulses and the bursts are separated by an inter-burst period that comprises zero pulses.

In embodiments, the energy source is configured to transmit a plurality of electrical impulses to the selected nerve according to a treatment paradigm. The treatment paradigm is sufficient to reduce inflammation in the brain of the user.

In embodiments, the treatment paradigm is sufficient to alter a microglia in a central nervous system of the user from a substantially pro-inflammatory state to a substantially non-inflammatory state. The treatment paradigm may be sufficient to reduce astrocytic activation with the central nervous system of the user. The treatment paradigm may be sufficient to increase glymphatic clearance of waste products within the brain of the user. The waste products comprise beta-amyloid, tau proteins and oxidative byproducts.

In one embodiment, the treatment paradigm comprises delivering the electrical impulses for at least 30 seconds within 4 hours of a commencement of sleep by the user, or for about 30 seconds to about 5 minutes within 3 hours, or 2 hours or 1 hour prior to commencement of sleep. The electrical impulse may be applied in a single dose for a time period of about 30 seconds and about 5 minutes, preferably about 90-150 seconds, or it may be applied in a series of doses each having a time period of about 30 seconds to about 3 minutes, preferably about 90-150 seconds in each dose. The series of doses may be applied every 5 to 30 minutes, or every 10 to 20 minutes, or every 15 minutes, for a period of at least 1 hour, or at least 2 hours or about 3 hours.

In embodiments, the device further comprises a housing, such as a handheld device, that may be operated by the user. The energy source is housed within the housing and the electrodes are attached to, or incorporated into, the housing.

The housing may contain the electronic components, signal generator and energy source (not shown) that are used to generate the signals that drive electrical impulses through the electrodes. However, in other embodiments, the electronic components that generate the signals may be in a separate housing or device, such as a mobile device. Furthermore, other embodiments may contain a single electrode or more than two electrodes.

The electrical impulse may also be sufficient to maintain and/or improve a cognitive performance of the user despite sleep deprivation. Maintaining or improving cognitive performance may include, but is not limited to, temporarily or permanently improving intelligence, learning capacity, memory retention, recall, mood, alertness and/or sleep efficiency in human beings. In various embodiments, the electrical impulse is sufficient to increase a memory of the user. In various embodiments, the electrical impulses is sufficient to reduce a fatigue of the user. In various embodiments, the electrical impulse is sufficient to increase a language acquisition skill of the user. In various embodiments, the electrical impulse is sufficient to increase an attention span of the user. In various embodiments, the electrical impulse is sufficient to increase a focus of the user.

In embodiments, the system further comprises a computer readable media comprising non-transitory computer executable instructions which, when executed by at least one electronic processor, causes the pulse generator to generate at least one electrical impulse and to transmit the at least one electrical impulse transcutaneously to the electrode. The electrode is configured to transmit the electrical impulses through the outer skin surface of the user to a selected nerve in the user adjacent to, or near, the target location.

In embodiments, the non-transitory computer readable media further comprises non-transitory computer executable instructions which, when executed by the at least one electronic processor, transmits parameters of the electrical impulse to the pulse generator.

In embodiments, the non-transitory computer readable media includes data and the pulse generator is configured to receive the data from the non-transitory computer readable media the data comprising a therapy regimen for treating a disorder in the user.

In embodiments, the non-transitory computer readable media includes data and the pulse generator is configured to receive the data from the non-transitory computer readable media the data comprising a therapy regimen for improving a general wellness of the user.

In embodiments, the non-transitory computer readable media includes data and the pulse generator is configured to receive the data from the non-transitory computer readable media the data comprising a therapy regimen for improving a cognitive performance of the user.

In embodiments, the non-transitory computer readable media further comprises non-transitory computer executable instructions which, when executed by the at least one electronic processor modulates a property of the electrical impulse.

In embodiments, the non-transitory computer readable media may be embodied in a software application configured for downloading onto a user interface. The software application controls parameters of the stimulator, which may be based on a physiological parameter of the patient and/or user status information related to the effectiveness of the sleep therapy.

In other embodiments, the device comprises a patch having at least one adhesive surface for attachment to the outer skin surface of the neck of the user. The electrodes are housed within the patch. The patch may further comprise a signal generator and an energy source for applying the electrical impulses through the electrodes to the vagus nerve. Alternatively, the patch may include a wireless receiver and associated electronics for wirelessly receiving the electrical impulse and/or the energy from the energy source.

The device may further comprise a controller coupled to the energy source and configured to transmit parameters for the stimulation protocol to the energy source. The controller and/or the energy source may be wirelessly coupled to the electrodes, or each other. Alternatively, the controller and the energy source may be housed within the patch or the handheld device.

In certain embodiments, the energy source is wirelessly coupled to the one or more electrodes. In other embodiments, the energy source is coupled to the electrodes directly with electrical connectors. In yet other embodiments, the energy source and the electrodes are housing within a handheld device that can be placed or attached against the outer surface of the user's neck.

In one such embodiment, the electrodes are adhered to the outer skin surface of the user's neck with a suitable adhesive. This allows the user to be treated without direct intervention (i.e., holding a device or the electrodes against the user's neck during stimulation). The system may further comprise an outer sheath or other wearable device, such as an insulating strip, a collar, or a garment, such as a turtleneck, a scarf, neck massager, neck pillow or the like, that functions to adhere or otherwise position the electrodes to the neck of the user. The electrodes may be housed within the wearable device, or positioned between the wearable device and the neck of the user.

In another aspect, a method for enhancing sleep comprises transmitting at least one electrical impulse transcutaneously from the electrode through the outer skin surface of the user to a selected nerve in the user adjacent to, or near, the target location and delivering one or more stimuli to a brain of the user. The stimuli comprises one or more of a visual stimuli, an auditory stimuli, an olfactory stimuli, a tactile stimuli or a combination thereof.

The nerve stimulation may be delivered to the user prior to sleep, during sleep or after the user has wakened. The sensory stimulation may be delivered to the user prior to sleep, during sleep or after the user has wakened. In an exemplary embodiment, the nerve stimulation is delivered prior to sleeping and the sensory stimulation is delivered during sleeping.

In embodiments, the method comprises delivering both auditory and visual stimuli to the user during sleep. In an exemplary embodiment, binaural tones or beats are delivered to the brain of the user. In embodiments, the binaural tones have carrier frequencies of about 10 Hz to about 100 Hz, or about 20 Hz to about 80 Hz, or about 30 Hz to about 50 Hz, or about 40 Hz. The binaural tones have a beat frequency of about 0.1 Hz to about 10 Hz, or about 0.2 Hz to about 2 Hz, or about 0.5 Hz to about 1.5 Hz, or about 1 Hz.

In embodiments, the method comprises delivering one or more light patterns to the user. In an exemplary embodiment, the light patterns are synchronized with the binaural tones. In certain embodiments, the light patterns may comprise waves that flicker at about 10 Hz to about 100 Hz, or about 20 Hz to about 80 Hz, or about 30 Hz to about 50 Hz, or about 40 Hz. In embodiments, the light waves may have a wavelength of about 580 nm to about 830 nm, or about 610 nm to about 750 nm or about 620 nm to about 640 nm.

In embodiments, the method further comprises detecting differential brainwave activity in the user and computing an effective brainwave frequency based on the detected brainwave activity. In an exemplary embodiment, the binaural tones are synchronized with the effective brainwave frequency.

In embodiments, the target nerve is the vagus nerve. In certain embodiments, the electrical impulse comprises pulses having a frequency of about 1 kHz to about 20 kHz. The electrical impulse may comprise bursts of pulses, with each burst having a frequency of about 1 to about 100 bursts per second and each pulse has a duration of about 50 to about 1000 microseconds in duration. The bursts each comprise about 2 to 20 pulses and the bursts are separated by an inter-burst period that comprises zero pulses.

In embodiments, the method comprises transmitting a plurality of electrical impulses to the selected nerve according to a treatment paradigm. The treatment paradigm is sufficient to reduce inflammation in the brain of the user. In embodiments, the treatment paradigm is sufficient to alter a microglia in a central nervous system of the user from a substantially pro-inflammatory state to a substantially non-inflammatory state. The treatment paradigm may be sufficient to reduce astrocytic activation with the central nervous system of the user. The treatment paradigm may be sufficient to increase glymphatic clearance of waste products within the brain of the user. The waste products comprise beta-amyloid, tau proteins and oxidative byproducts.

In one embodiment, the treatment paradigm comprises delivering the electrical impulses for at least 30 seconds within 4 hours of a commencement of sleep by the user, or for about 30 seconds to about 5 minutes within 3 hours, or 2 hours or 1 hour prior to commencement of sleep. The electrical impulse may be applied in a single dose for a time period of about 30 seconds and about 5 minutes, preferably about 90-150 seconds, or it may be applied in a series of doses each having a time period of about 30 seconds to about 3 minutes, preferably about 90-150 seconds in each dose. The series of doses may be applied every 5 to 30 minutes, or every 10 to 20 minutes, or every 15 minutes, for a period of at least 1 hour, or at least 2 hours or about 3 hours.

Various technologies for stimulating nerves are more completely described in the following detailed description, with reference to the drawings provided herewith, and in claims appended hereto. Other aspects, features, advantages, etc. will become apparent to one skilled in the art when the description is taken in conjunction with the accompanying drawings.

### INCORPORATION BY REFERENCE

Hereby, all issued patents, published patent applications, and non-patent publications that are mentioned in this specification are herein incorporated by reference in their entirety for all purposes as if copied and pasted herein, to the same extent as if each individual issued patent, published patent application, or non-patent publication were specifically and individually indicated to be incorporated by reference and copied and pasted herein.

### DESCRIPTION OF DRAWINGS

FIGS. 1A-1B provide schematic diagrams for the operation of: (FIG. 1A) a conventional evoked potential measurement device and (FIG. 1B) a closed loop nerve stimulator, evoked potential measurement device and/or biofeedback;

FIG. 1C illustrates a schematic view of one embodiment of a nerve modulating system;

FIG. 2A shows an embodiment of an electrical voltage/current profile for stimulating and/or modulating impulses that are applied to a nerve;

FIG. 2B illustrates one burst of an electrical waveform for stimulating and/or modulating a nerve;

FIG. 2C illustrates an embodiment of two successive bursts of the waveform of FIG. 2B;

FIG. 3A is a perspective view of a stimulator;

FIG. 3B is a perspective view of the stimulator of FIG. 3A flipped upside down;

FIG. 3C is a perspective view of another embodiment of a stimulator with a cover for protecting the electrodes;

FIG. 3D is a perspective view of the stimulator of FIG. 3C with the cover positioned to expose the electrodes;

FIG. 4 is a perspective view of another embodiment of a stimulator;

FIG. 5A is a perspective view of another embodiment of a stimulator in the closed position;

FIG. 5B is a perspective view of the stimulator of FIG. 5A in an open position;

FIG. 5C is a perspective view of another side of the stimulator of FIG. 5A in the closed position;

FIG. 5D is a perspective view of another side of the stimulator of FIG. 5A in the open position;

FIG. 6 illustrates a stimulator when positioned to stimulate a vagus nerve in a patient's neck;

FIG. 7A is a front view of another embodiment of a stimulator;

FIG. 7B is a side view of the stimulator of FIG. 7A;

FIG. 8A is a front view of another embodiment of a stimulator;

FIG. 8B is a back view of the stimulator shown in FIG. 8A;

FIG. 8C is a side view of the stimulator shown in FIG. 8A;

FIG. 9 shows an expanded diagram of an embodiment of a control unit;

FIG. 10 illustrates an embodiment of an approximate position of a stimulator when used to stimulate a right vagus nerve in a neck of an adult patient;

FIG. 11 illustrates an embodiment of an approximate position of a stimulator when used to stimulate a right vagus nerve in a neck of a child who wears a collar to hold the stimulator;

FIG. 12 illustrates a system for modulating the vagus nerve;

FIG. 13 illustrates a patch stimulator device for attaching to a skin surface of a patient;

FIG. 14A is a top view of an electrode array;

FIG. 14B is an exploded side view of the electrode array of FIG. 14A;

FIGS. 15A-15I are schematic views of a user interface generated by a downloadable software program;

FIG. 16 is a graph of the loss of cognitive performance based on the number of hours of sleep per night;

FIG. 17 is a graph illustrating permanent pain state and enhanced responsiveness to pain-triggering stimuli upon repeated administrations of inflammatory media to the dura of the brain;

FIG. 18 is a front view of one embodiment of a device for applying stimuli to a user during sleep;

FIG. 19 is a side view of the device of FIG. 18;

FIG. 20 is a block diagram of a system for measuring brainwave activity to compute an effective brainwave frequency and for delivering visual and auditory stimuli to the brain that is synchronized with the effective brainwave frequency;

FIG. 21 is a flowchart of a closed-loop control algorithm for the system of FIG. 20;

FIG. 22 is an alternative embodiment of a device for applying stimuli to a user during sleep;

FIG. 23 schematically illustrates a system for detecting physiological and environmental parameters and adjusting the electrical impulse based on those parameters; and

FIGS. 24A and 24B are graphs illustrating relative voltage versus time of vagal nerve stimulation.

### DETAILED DESCRIPTION

Systems, devices and methods are provided for delivering a combination of electrical impulses (and/or fields) and various stimuli to bodily tissues for various purposes. In certain aspects, the systems and methods are particularly useful for enhancing sleep quantity, quality, and/or efficiency to promote faster sleep onset, more effective time in slow wave sleep, enhanced neurological health and reduced required sleep duration. The combined therapy described herein increases the brain's capacity to clear metabolic and neurotoxic material (waste removal), facilitates neuroplasticity to affect brain network optimization (learning and memory consolidation), and restores brain energy and neurotransmitter levels (neurometabolic restoration). The systems and methods may also be useful for improving a cognitive performance of the user by maintaining cognitive function during sleep deprivation and/or temporarily or permanently improving intelligence, learning capacity, memory retention, recall, mood and/or alertness.

In certain embodiments, the electrical impulses are delivered non-invasively to the target nerve. A procedure can be understood as being non-invasive when no break in the skin (or other surface of the body, such as a wound bed) is created through use of the method, and when there is no contact with an internal body cavity beyond a body orifice (e.g., beyond the mouth or beyond the external auditory meatus of the ear). In some ways, such non-invasive procedures can be distinguished from some invasive procedures (including minimally invasive procedures) in that the invasive procedures insert a substance or device into or through the skin (or other surface of the body, such as a wound bed) or into an internal body cavity beyond a body orifice.

In particular, the devices can transmit energy to, or in close proximity to, a selected nerve of the user in order to stimulate, block and/or modulate electrophysiological signals in that nerve. In some embodiments, one or more electrodes applied to the skin of the user generate currents within the tissue of the user. This may enable production and application of the electrical impulses so as to interact with the signals of one or more nerves, in order to achieve the therapeutic result.

In some embodiments, methods and devices are specifically designed for stimulation in or around a vagus nerve, with devices positioned non-invasively on or near a user's neck to target the cervical branch of the vagus nerve and/or in or around the auricular branch of the vagus nerve of the user (i.e., within the ear, on the surface of the ear, or on the user's head or upper neck near the auricular nerve). However, it will be recognized that some of the treatment paradigms described herein can be used with a variety of different vagal nerve stimulators, including implantable and/or percutaneous stimulation devices.

The methods and devices disclosed herein may also be used for non-medical purposes, such as reducing stress, enhancing relaxation, improving energy, concentration and mood, increasing mental or physical performance, promoting mental health, recovery and wellness and generally improving the health and wellbeing of a user.

The stimuli delivered to the user may include, but is not limited to, visual stimuli, auditory stimuli, tactile stimuli, olfactory stimuli, physical stimuli and combinations thereof. In certain embodiments, the stimuli is a combination of auditory and visual stimuli.

The electrical stimulation may be delivered to the user before sleep, during sleep or after waking. The auditory and visual stimuli may also be delivered to the user before sleep, during sleep or after waking. In certain embodiments, the electrical stimulation is delivered prior to sleep and the auditory and visual stimuli is delivered while the user is sleeping. In an exemplary embodiment, the electrical stimulation primes the brain for the auditory and visual stimuli by enhancing neurotoxic waste clearance and optimizing the neural network for the auditory and visual stimuli.

In an exemplary embodiment, the system comprises a revolutionary multi-modal sleep system that combines synergistic closed-loop intra-sleep auditory and visual sensory stimulation with potentiating pre-sleep neuromodulation (Multimodal Optimization and Restoration Platform for Homeostatic Enhancement Under Sleep-restriction or MORPHEUS). MORPHEUS achieves restorative benefit from sleep restriction by maximizing glymphatic flow while enhancing neuroplasticity and neurotransmitter synthesis. The physical form of MORPHEUS is a combination headband and portable hand-held device. The headband incorporates closed-loop, EEG-controlled, sleep stage-coupled auditory binaural beats (BNB) and frequency- and intensity-optimized photomodulation technologies. The hand-held device delivers non-invasive vagus nerve stimulation (nVNS).

The sleep-modulating interventions of MORPHEUS were selected because of their potential to synergize and thereby reduce the previously described deficits caused by sleep restriction (including extreme conditions defined by 3 hr/night for 7 days). Repeated days of sleep restriction promote an elevated drive to generate slow wave sleep (SWS)1,3 when peak glymphatic clearance is typically maximized, however, the concurrent activation of glia into inflammatory postures tends to impair glymphatic clearance efficiency10. That is, expanded SWS is a physiologic response to the critical need for clearance of neurotoxic waste, but is impaired by inflammatory cytokine expression that reduces the efficiency of the glymphatic flow. This expanded SWS typically comes at the expense of rapid eye movement (REM) sleep, which is known to play important roles in memory consolidation, and leading to poor concentration, decision-making, and emotional regulation. MORPHEUS synergistically uses auditory binaural tones and precisely synched 40 Hz gamma stimulating photomodulation in a closed-loop system that employs active monitoring of sleep stages using real-time EEG and motion sensing to restore cognitive performance. To potentiate these effects, however, there is a need to reorient microglia and astrocytes into anon-inflammatory posture. To accomplish this, MORPHEUS employs nVNS pre-conditioning, reducing inflammatory signaling, and facilitating efficient glymphatic flow and changes to macro- and micro-sleep architecture to facilitate memory consolidation processes. More specifically, together, these interventions will reinforce SWS while providing neurotoxin-clearing gamma stimulation through the carrier tone, and restore benefits normally associated with REM sleep. More specifically, with respect to each component of MORPHEUS:

The auditory intervention of MORPHEUS is binaural beats (BNB). More specifically, BNB leverage the neurological phenomenon in which a virtual tone (typically the beat that occurs through constructive and destructive interference between two sinusoidal tones of modestly different frequency) is perceived by the brain, and neurologic activity entrains to this virtual beat. It is important to note that this remarkable phenomenon occurs even when the two signals are not interacting anywhere but within the brain, i.e., the different frequencies are presented through headphones to different ears. This effect results from integrating signals from the two ears in the Medial Superior Olive (MSO) of the brain stem. It has previously been shown that BNB of the appropriate frequency can augments glymphatic flow when the virtual beat of the BNB entrains brain activity into specific sleep stages through frequency-following responses by entraining delta (1-4 Hz) activity to enhance SWS. Therefore, glymphatic system flow peaks during SWS facilitated by brain entrainment of BNB.

“[S]leep deficit causes the opening of the blood-brain barrier (BBB) to inflammatory mediators and immune cells in both humans and rodents . . . Chronic sleep restriction promotes astrocytic phagocytosis of synaptic elements and microglia activation, i.e., the brain begins to “eat” itself.” (Semyachkina-Glushkovskaya, Oxana, Ivan Fedosov, Thomas Penzel, Dongyu Li, Tingting Yu, Valeria Telnova, Elmira Kaybeleva et al. “Brain waste removal system and sleep: photobiomodulation as an innovative strategy for night therapy of brain diseases.” International journal of molecular sciences 24, no. 4 (2023): 3221.) Sleep restriction activates microglia into an inflammatory state, which has been shown to cause cognitive impairment, which impairment can be restored if inflammatory cytokine signaling is blocked. (Kincheski, G., et al., “Chronic sleep restriction promotes brain inflammation and synapse loss, and potentiates memory impairment induced by amyloid-β oligomers in mice.” Brain, behavior, and immunity 64 (2017): 140-151.) Inflammatory signaling impairs glymphatic clearance mechanisms. (Wafford, K., “Aberrant waste disposal in neurodegeneration: why improved sleep could be the solution.” Cerebral Circulation-Cognition and Behavior 2 (2021): 100025.) Thus, sleep deprivation is linked to inflammation, BBB disruption, and glymphatic dysfunction (Voumvourakis, K., et al., “The dynamic relationship between the glymphatic system, aging, memory, and sleep.” Biomedicines 11, no. 8 (2023): 2092.)

The activation of microglia has also been associated with reduced REM sleep dysfunction. This association has also been observed in conjunction with cortical cholinergic dysfunction. (Stær, Kristian, Alex Iranzo, Morten G. Stokholm, Karen Østergaard, Mónica Serradell, Marit Otto, Kristina B. Svendsen et al. “Cortical cholinergic dysfunction correlates with microglial activation in the substantia innominata in REM sleep behavior disorder.” Parkinsonism & Related Disorders 81 (2020): 89-93.) This observation suggests that enhanced cholinergic signaling may enhance REM sleep, and by extension the efficacy of interventions that seek to enhance REM sleep macro- and micro-architecture.

Photomodulation involves the use of visual signals, e.g., flashing lights of specific wavelengths and/or frequencies, to alter broader neurological activity within the brain. According to Murdock (Murdock, Mitchell H., Cheng-Yi Yang, Na Sun, Ping-Chieh Pao, Cristina Blanco-Duque, Martin C. Kahn, TaeHyun Kim et al. “Multisensory gamma stimulation promotes glymphatic clearance of amyloid.” Nature 627, no. 8002 (2024): 149-156.) “multisensory 40 Hz stimulation promotes the influx of cerebrospinal fluid and the efflux of interstitial fluid in the cortex . . . which was associated with increased aquaporin-4 polarization along astrocytic endfeet, dilated meningeal lymphatic vessels, and amyloid accumulation in cervical lymph nodes.” In 2024, Sun, et al., extended this by reporting that “brief 40 Hz light flickering robustly enhanced glymphatic influx and efflux in a frequency dependent manner . . . attributed to increased AQP4 polarized expression in astrocyte endfeet and enhanced vasomotion. Furthermore, we identified adenosine-A2AR signaling as a molecular underpinning of 40 Hz flickering-induced enhancement of glymphatic flow.” (Sun, Xiaoting, Liliana Dias, Chenlei Peng, Ziyi Zhang, Haoting Ge, Zejun Wang, Jiayi Jin et al. “40 Hz light flickering facilitates the glymphatic flow via adenosine signaling in mice.” Cell Discovery 10, no. 1 (2024): 81.)

In a recent review of photomodulation use for the enhancement of neurotoxic waste (amyloid) clearance, efficacy was observed in the range of red light (˜570-810 nm). (Semyachkina-Glushkovskaya, Oxana, Thomas Penzel, Mikhail Poluektov, Ivan Fedosov, Maria Tzoy, Andrey Terskov, Inna Blokhina, Viktor Sidorov, and Jurgen Kurths. “Phototherapy of Alzheimer's disease: photostimulation of brain lymphatics during sleep: a systematic review.” International journal of molecular sciences 24, no. 13 (2023): 10946.) More specifically, animal AD studies with photomodulation (40 Hz at 630+/−30 nm) have demonstrated attenuated Aβ and Tau pathology, with most studies pointing towards enhancement of degradation/clearance mechanisms in the brain.19 Given that 650 nm light has also been shown to enhance the function of cytochrome c oxidase (via dissociation of nitric oxide molecules that occlude the active site of the enzyme), thereby reducing oxidative stress associated with sleep restriction, MORPHEUS utilizes 650 nm light flickering at 40 Hz.

In addition to its waste clearance potentiation effects, Chan, et al. demonstrated that 40 Hz sensory stimulation reliably induces EEG-verified gamma oscillations in Alzheimer's patients, supporting its potential as a neuromodulatory frequency17. More specifically, with respect to sleep architecture and brain network optimization, gamma (40 Hz) photomodulation induces occipital gamma spikes (25-50 Hz)18 to stimulate REM characteristics in the visual cortex and yield REM-associated benefits. Interestingly, the effects of elevated inflammatory markers has been correlated with inhibited gamma activity in individuals with chronic neurosensory conditions, e.g., tinnitus. “We found a significant negative correlation between CRP and gamma power in the orbitofrontal cortex in tinnitus patients (p<0.001), pointing to a deactivation of the orbitofrontal cortex when CRP was high. (Becker, Linda, Antonia Keck, Nicolas Rohleder, and Nadia Müller-Voggel. “Higher peripheral inflammation is associated with lower orbitofrontal gamma power in chronic tinnitus.” Frontiers in Behavioral Neuroscience 16 (2022): 883926.)

”[S]leep (and its disturbance/loss) modulates the immune system by modulating norepinephrine levels in the brain (NE is a key modulator of microglia) and vice versa (Ma, Chenyan, Bing Li, Daniel Silverman, Xinlu Ding, Anan Li, Chi Xiao, Ganghua Huang et al. “Microglia regulate sleep through calcium-dependent modulation of norepinephrine transmission.” Nature Neuroscience 27, no. 2 (2024): 249-258.) In fact, REM sleep requires a reduction in noradrenergic activity, while, conversely, increases in norepinephrine levels upon REMS loss is responsible for many of the symptoms of sleep disruption. The increased levels of NA during REMS loss may cause neuroinflammation possibly by glial activation.” (Mehta, Rachna, Rohosen Bhattacharya, and Birendra Nath Mallick. “Sleep and neuroimmunomodulation for maintenance of optimum brain function: role of noradrenaline.” Brain sciences 12, no. 12 (2022): 1725.)

Similarly, “inflammation-driven SWA correlates with delirium severity in humans . . . [and] delirium, cognitive decline, and dementia are profound cognitive disorders associated with inflammation and changes in SWA.” (Sultan, Ziyad W., Elizabeth R. Jaeckel, Bryan M. Krause, Sean M. Grady, Caitlin A. Murphy, Robert D. Sanders, and Matthew I. Banks. “Electrophysiological signatures of acute systemic lipopolysaccharide-induced inflammation: potential implications for delirium science.” British Journal of Anaesthesia 126, no. 5 (2021): 996-1008.)

Microglial activity is also associated with micro-sleep architecture disruption. “CSF measures of glial activation were associated with frontal decreases in fast spindle activity.” (Neylan, Thomas C., and Christine M. Walsh. “Sleep spindles, tau, and neurodegeneration.” Sleep 45, no. 9 (2022): zsac161.)

Activated microglia express inflammatory cytokines, several of which have been shown to disrupt enzymes (tryptophan and tyrosine hydrolases) associated with the synthesis of key neurotransmitters (norepinephrine, serotonin, and dopamine). (Mehta, R., et al., “Sleep and neuroimmunomodulation for maintenance of optimum brain function: role of noradrenaline.” Brain sciences 12, no. 12 (2022): 1725.) Tumor necrosis factor-alpha (TNF-α) has been shown to upregulate transporters that remove extracellular neurotransmitters, like serotonin, reducing the influence of these important neurotransmitters influencing mood, motivation, pain perception, and stress resilience. (Lichtblau, Nicole, Frank M. Schmidt, Robert Schumann, Kenneth C. Kirkby, and Hubertus Himmerich. “Cytokines as biomarkers in depressive disorder: current standing and prospects.” International review of psychiatry 25, no. 5 (2013): 592-603.) TNF-α and interleukin 1- beta (IL-1β) have been shown to promote excitotoxicity through inhibition (and even reversal) of glutamate transport in astrocytes, and decrease GABA receptor density while enhancing AMPA and NMDA receptor populations on neurons. (Olmos, G., et al., “Tumor necrosis factor alpha: a link between neuroinflammation and excitotoxicity.” Mediators of inflammation 2014, no. 1 (2014): 861231.)

Sleep restriction-activated glia, especially astrocytes, can damage existing neural connections in high use, in an attempt to reduce the metabolic waste being generated. In a mouse model of chronic sleep restriction, cognitive deficits were evidenced by a decline in learning and memory of 12% and an 18% reduction in novel object recognition, which were observed to be associated with increases in astrocyte-mediated synapse elimination. (Zhai, Q., et al., “Reducing complement activation during sleep deprivation yields cognitive improvement by dexmedetomidine.” British journal of anesthesia 131, no. 3 (2023): 542-555.) Astrocytic phagocytosis, mainly of presynaptic components of large synapses, increased after both acute and chronic sleep loss suggesting that it may represent the brain's response to the increase in synaptic activity associated with prolonged wake, clearing worn components of heavily used synapses. (Bellesi, M., et al., “Sleep loss promotes astrocytic phagocytosis and microglial activation in mouse cerebral cortex.” Journal of Neuroscience 37, no. 21 (2017): 5263-5273).

If microglial and astrocytic activation can disrupt the efficiency of glymphatic clearance, sleep stages, neurotransmitter levels and functions, macro- and micro-sleep architecture, and even trigger degradation of neural connectivity, then preventing that activation may be a critical underlying function to be incorporated into an effective sleep modulating intervention.

VNS activates the release of norepinephrine from the locus coeruleus. The elevated norepinephrine levels, however, is very brief, lasting only as long as the stimulation is active. During this period, the dorsal Raphe nucleus is activated, triggering elevations in serotonin synthesis and release which can last for at least 30 minutes, and likely substantially longer. Similarly, the activation of the locus coeruleus leads to an activation of the nucleus basalis of Meynert, which leads to a widespread upregulation in acetylcholine release (through synaptic release, but primarily through volume transmission into the ISF via the 86-93% of cholinergic boutons that do not make synaptic contact) (Sfera, Adonis, and Carolina Osorio. “Water for thought: is there a role for aquaporin channels in delirium?” Frontiers in Psychiatry 5 (2014): 57.) which lasts for hours. This resolves the surface level paradox of why a mechanism that elevates norepinephrine levels, which are associated with the waking state and are believed to result in significantly impaired glymphatic activity, including reduced glymphatic channels, reduced AQP-4 expression on astrocytes, and diminished interstitial volume, would enhance glymphatic flow.

In fact, Sfera, et al. wrote, “[s]ince VT represents a large proportion of ACh signaling, it was hypothesized that it may support the sustained and widespread neural functions such as cognition, attention, awareness, and sleep.” This acetylcholine release has multiple effects that are relevant to sleep.

For example, elevated acetylcholine levels have been shown to enhance glymphatic flow and the clearance of neurotoxic waste, and have been implicated in the observed efficacy of 40 Hz gamma entrainment modalities. As reported by Lavoie, e al. In 2024 “Gamma Entrainment Using Sensory Stimuli (GENUS) promotes amyloid clearance via increased perivascular cerebral spinal fluid (CSF) flux . . . GENUS promoted cortical acetylcholine release, vascular dilation, vasomotion and perivascular clearance. Inhibiting cholinergic signaling abolished the effects of GENUS, including the promotion of arterial pulsatility, periarterial CSF influx, and the reduction of cortical amyloid levels. Our findings establish cholinergic signaling as an essential component of the brain's ability to promote perivascular amyloid clearance” (Lavoie, Nicolas, Cristina Blanco-Duque, Martin Kahn, Hiba Nawaid, Anjanet Loon, Alexander Seguin, Ravikiran Raju, Alexis Davison, Cheng-yi Yang, and Li-Huei Tsai. “The role of cholinergic signaling in multisensory gamma stimulation induced perivascular clearance of amyloid.” bioRxiv (2024): 2024-11.)

In 2003, Armitage, et al., reported on the effects of VNS on sleep macro-architecture among depression patients who received implanted vagus nerve simulators for the treatment of MDD, in whom significant sleep dysfunction was also apparent. The authors reported, “effects of VNS on strength or amplitude of sleep EEG rhythms were dramatic and significant. The average amplitude of all rhythms, except for interhemispheric q, more than doubled on VNS—a finding supported by the large overall treatment effect . . . The most dramatic effects were found for the amplitude of rhythms with more than a 200% increase in power after VNS.” (Armitage, Roseanne, Mustafa Husain, Robert Hoffmann, and A. John Rush. “The effects of vagus nerve stimulation on sleep EEG in depression: a preliminary report.” Journal of psychosomatic research 54, no. 5 (2003): 475-482.) This observation was also confirmed in 2005 by Hallbrook, et al., who found that among children with epilepsy who received vagus nerve stimulators, “VNS induces a significant increase in slow wave sleep (SWS) and a decrease in sleep latency and in stage 1 sleep.” (Hallböök, Tove, Johan Lundgren, Sven Köhler, Gösta Blennow, Lars-Göran Strömblad, and Ingmar Rosén. “Beneficial effects on sleep of vagus nerve stimulation in children with therapy resistant epilepsy.” European Journal of Paediatric Neurology 9, no. 6 (2005): 399-407.)

Early observations of cognitive and memory enhancing benefits of vagus nerve stimulation arose from the treatment of individuals with severe refractory epilepsy. These reports were, however, inconsistent, and small case series and pilot studies produced limited evidence of an effect. However, with the expansion of the indication for implanted VNS into the field of refractory depression, more consistent reports began to populate the literature. As one group found, “vagus nerve stimulation patients significantly improved on cognitive and clinical measures. Learning and memory improved rapidly after 1 month of stimulation, and other cognitive functions improved gradually over time. Cognitive improvements were sustained up to 2 years of treatment.” (Jodoin, Véronique Desbeaumes, Frangois Richer, Jean-Philippe Miron, Marie-Pierre Fournier-Gosselin, and Paul Lespérance. “Long-term sustained cognitive benefits of vagus nerve stimulation in refractory depression.” The journal of ECT 34, no. 4 (2018): 283-290.) For nearly two decades, the extreme expense of VNS surgery relegated the use of the procedure to the most severely affected patients, clouding the ability to discern the true potential of VNS with respect to cognitive enhancement.

The advancement of non-invasive VNS technologies (auricular and cervical) dramatically expanded the opportunity to study the cognitive enhancing potential of VNS among relatively healthy individuals. Over the past several years, several important findings have been reported that suggest VNS can provide memory and learning benefits to otherwise healthy individuals. In one study, the use of cervical non-invasive VNS among individuals who had been sleep deprived for 34 hours found that VNS treated individuals “performed significantly better on arousal, multi-tasking, and reported significantly lower fatigue ratings compared to sham for the duration of the study.” (McIntire, Lindsey K., R. Andy McKinley, Chuck Goodyear, John P. McIntire, and Rebecca D. Brown. “Cervical transcutaneous vagal nerve stimulation (ctVNS) improves human cognitive performance under sleep deprivation stress.” Communications biology 4, no. 1 (2021): 634.). The same investigators wrote: “Vagal Nerve Stimulation (VNS) stimulation has been shown to significantly improve memory and performance of cognitive tasks in both rats and humans . . . VNS has been shown to activate . . . the hippocampus and amygdala. These regions of the brain are also known to be critically important for learning and comprehension, as well as cognition generally. Human subjects receiving VNS have shown specific enhancements in comprehension, leading to enhanced decision making. VNS has also been shown to increase neuronal plasticity in humans.” (McIntire, Lindsey, Chuck Goodyear, and Andy McKinley. “Peripheral nerve stimulation to augment human analyst performance.” In 2019 IEEE Research and Applications of Photonics in Defense Conference (RAPID), pp. 1-3. IEEE, 2019.).

Subsequent findings further support the assertion that VNS can enhance learning and memory. “Along with neurological and psychiatric indications, clinical and preclinical studies suggest that VNS can improve memory . . . VNS-induced memory improvements [are] related to the hippocampus, the main area implicated in memory acquisition.” (Olsen, Laura K., Ernesto Solis Jr, Lindsey K. McIntire, and Candice N. Hatcher-Solis. “Vagus nerve stimulation: mechanisms and factors involved in memory enhancement.” Frontiers in human neuroscience 17 (2023): 1152064.). Most recently, improvement in cognitive performance was revealed at the Defense Language Institute, where researchers found non-invasive VNS “[I]n a 5 day second-language vocabulary acquisition protocol among highly selected career linguists at the US Department of Defense's premier language school . . . tcVNS produced accelerated recall performance . . . maintained across a 24 h retention interval . . . tcVNS also produced fatigue-mitigating and focus-promoting effects . . . [T]he current and the previous findings supporting tVNS' efficacy on performance, training enhancement, and fatigue mitigation . . . tcVNS to be an effective learning acceleration tool that can be utilized at language-teaching and other institutions focused on intensive training of cognitive skills.” (Miyatsu, Toshiya, Vanessa Oviedo, Jajaira Reynaga, Valerie P. Karuzis, David Martinez, Polly O'Rourke, Melissa Key et al. “Transcutaneous cervical vagus nerve stimulation enhances second-language vocabulary acquisition while simultaneously mitigating fatigue and promoting focus.” Scientific Reports 14, no. 1 (2024): 17177.)

The use of nVNS as described herein is intended to reduce microglial and astrocytic activation (i.e., inflammation), potentiating the effects of the intra-sleep interventions of the MORPHEUS system (i.e., the closed-loop EEG coordinated neuromodulatory auditory and visual signals). These effects are expected to optimize glymphatic clearance and facilitate macro- and micro-sleep architecture changes to enhance help memory consolidation. for the above reasons, however, expected that the direct benefits of nVNS pre-conditioning will also accrue to users, enhancing cognitive performance.

Finally, one of the more concerning aspects of the original sleep restriction study is the finding that individuals subjected to chronic sleep restriction (whether 3, 5, or even 7 hours) do not experience a complete recovery to their baseline psychomotovigilance (PVT) performance level, even after 3 nights of recovery sleep. This is reminiscent of the semi-permanent (and in some cases fully permanent) transitioning of animals to a chronic pain state using repeated inflammatory insults. (Oshinsky, Michael L., Angela L. Murphy, Hugh Hekierski Jr, Marnie Cooper, and Bruce J. Simon. “Noninvasive vagus nerve stimulation as treatment for trigeminal allodynia.” Pain 155, no. 5 (2014): 1037-1042., Hawkins, Jordan L., Lauren E. Cornelison, Brian A. Blankenship, and Paul L. Durham. “Vagus nerve stimulation inhibits trigeminal nociception in a rodent model of episodic migraine.” Pain reports 2, no. 6 (2017): e628.) The use of periodic vagus nerve stimulation throughout the sensitization period (e.g., during an 8-day period of sensitization caused by injection of complete Freund's adjuvant into the trapezius muscles) prevented this sensitization from occurring, effectively shielding the animal from the long-term transition to chronic pain. (Cornelison, Lauren E., Jordan L. Hawkins, Sara E. Woodman, and Paul L. Durham. “Noninvasive vagus nerve stimulation and morphine transiently inhibit trigeminal pain signaling in a chronic headache model.” Pain Reports 5, no. 6 (2020): e881.) The mechanism by which this shielding is believed to occur is the inhibition of microglial priming, which may be the underlying reason for the impaired recovery observed after chronic sleep restriction. It is our intent to measure whether daily nVNS preconditioning may have the ability to prevent this priming and thus permit subjects to experience a full return to baseline PVT performance within the 3-day recovery period.

The systems and methods described herein may also be configured to increase a user's cognitive performance, skill proficiency, judgement, vigilance, attention and memory by inducing neuroplasticity, improving neurobehavioral outcomes, enhancing focus and mitigating fatigue. In some aspects, improving sleep efficiency will result in such improvements. In other aspects, the systems and methods may provide these improvements independently of the improved sleep efficiency. Examples include enhancing learning and skill acquisition, such as second language learning vocabulary acquisition and/or enhancing cognitive performance in extreme environments, such as extreme stressors, multiday transoceanic operational and logistic flights, long duration remotely piloted aircraft missions and the like.

In other embodiments, the systems and methods are particularly useful for temporarily or permanently improving intelligence, learning capacity, memory retention, recall, mood, alertness and/or sleep efficiency in human beings. In some aspects, improving sleep efficiency will result in such improvements. In other aspects, the systems and methods may provide these improvements independently of the improved sleep efficiency. In certain embodiments, the methods and devices enhance neurostructural development over a period of time by increasing neurogenesis, neuronal plasticity and/or neural connectivity efficiency, and/or by improving the chemical microenvironment of the evolving neural network. In some embodiments, these enhancements may provide temporary improvement to enable an individual to, for example, accomplish a particular task while under duress, sleep deprivation, stress, anxiety or the like. In other embodiments, these changes permanently enhance the intelligence, emotional stability, and overall brain health of the treated individual.

In one example of the above embodiments, the systems and methods may be used to sense, assess and augment cognitive performance in operational environments, including reducing or mitigated stress/fatigue in extreme environments to augment performance. For example, the system and methods may increase cognitive performance in aerospace environments such as multi-day transoceanic operational and logistic flights as well as long duration remotely piloted aircraft missions. Fatigue resulting from these extreme stressors can evolve into chronic health problems, and cause decrements in judgement and vigilance resulting in severe aviation mishaps.

The effectiveness of the neural network may be increased through neurogenesis, or the creation of more neurons in the brain. Alternatively, or in addition, and depending on the timing thereof within the framework of development, the effectiveness may be enhanced by increasing a connectivity of neurons within the brain of the individual and/or increasing the effective pruning of connections, or enhancing a neuronal plasticity within the brain of the individual. Neuronal plasticity is generally defined as the ability of the brain to change its structure and/or function in response to previous experience. It is essential for the establishment and refinement of neural networks during development and the formation of memory traces, the acquisition of specific skills and the storage of information.

In certain embodiments, the effectiveness of the neural network is increased sufficiently to temporarily or permanently improve the intelligence, learning capacity, memory retention, recall, mood, alertness and/or sleep efficiency in the individual. These enhancements may provide temporary improvement to enable an individual to, for example, accomplish a particular task while under duress, sleep deprivation or the like. In other embodiments, these changes permanently enhance the intelligence, emotional stability, and overall brain health of the treated individual.

In other embodiments, the systems and methods are useful for directly or indirectly increasing an activity of telomerase within the user. Increasing telomerase activity protects the end of the chromosome from DNA damage or from fusion with neighboring chromosomes, thereby maintaining the length of the telomeres or at least inhibiting the natural reduction of telomeres. Inhibiting the reduction of telomere lengths may potentially increase the lifespan or health span on an individual. Lifespan as defined herein as the duration of life of an individual. Health span is defined herein as the period of one's life that one is substantially free from serious disease. A disease is “serious” if it is a leading cause of death, which includes heart disease, lung, colorectal, breast or prostate cancer, COPD, stroke, lower respiratory infections, Alzheimer's disease and Type 2 diabetes. A more complete description of these embodiments can be found in U.S. patent application Ser. No. 17/731,393, filed Apr. 28, 2022, the complete disclosure of which is incorporated herein by reference for all purposes.

In other embodiments, the systems and methods are useful for treating user's suffering from systemic inflammation or chronic stress, anxiety or other disorders that invoke a chronic stress response resulting from elevated cortisol levels. These elevated cortisol levels may contribute to accelerated shortening of telomere lengths in cellular DNA, such as chronic pain, tumors or other disorders of the pituitary gland, depression, mood disorders, fear, perceived threats to safety, status or well-being, fatigue, irritability, headache, intestinal problems, increased blood pressure, poor sleep and the like. Reduction of these telomere lengths may potentially shorten the overall longevity and/or health span of the user.

Cortisol is a potent anti-inflammatory that functions to mobilize glucose reserves for energy and modulate inflammation. Cortisol also may facilitate the consolidation of fear-based memories for future survival and avoidance of danger. Although short-term stress may be adaptive, maladaptive responses (e.g., magnification, rumination, helplessness) to pain or non-pain-related stressors may intensify cortisol secretion and condition a sensitized physiologic stress response that is readily recruited. Ultimately, a prolonged or exaggerated stress response may perpetuate cortisol dysfunction, widespread inflammation, and pain. Stress may be unavoidable in life, and challenges are inherent to success; however, humans have the capability to modify what they perceive as stressful and how they respond to it. Exaggerated psychological responses (ego, catastrophizing) following maladaptive cognitive appraisals of potential stressors as threatening may exacerbate cortisol secretion and facilitate the consolidation of fear-based memories of pain or non-pain-related stressors; however, coping, cognitive reappraisal, or confrontation of stressors may minimize cortisol secretion and prevent chronic, recurrent pain.

Studies have shown that heightened cortisol responsivity to psychological stress is associated with accelerated cellular aging as indexed by leukocyte telomere length. This indicates that heightened cortisol responsivity is not simply a consequence of more advanced cellular aging but may contribute to the cellular aging process. Cortisol also suppresses telomerase activation in immune system cells so that telomeres are no longer protected during cell division and become progressively shorter. This leads to early cell aging and distorted replicas of the original cell that could lead to cancer and other diseases.

Applying vagal nerve stimulation to a user with a specific treatment protocols described herein may reduce the level of circulating cortisol in the user. This reduction in chronic cortisol levels may alter the chronic stress response, alleviating the many symptoms associated with such response. In addition, vagal nerve stimulation with the specific stimulation methods and devices described herein may increase an activity of telomerase within the user. Increasing telomerase activity protects the end of the chromosome from DNA damage or from fusion with neighboring chromosomes, thereby maintaining the length of the telomeres or at least inhibiting the natural reduction of telomeres.

In certain embodiments, the electrical impulse and the stimulation protocol are sufficient to modulate the vagus nerve to reduce a cortisol level within the user, particularly the amount of circulating cortisol within the user. Thus, devices and methods provided herein increase the ability of the parasympathetic nerve to adapt to upward regulation and stress. This not only provides a mechanism for stress control, but mitigates the impact of chronic stress by reducing the levels of circulating cortisol. Reducing these levels of cortisol deaccelerates telomere shortening and thus the ageing process. A more complete description of these embodiments can be found in U.S. patent application Ser. No. 17/844,368, filed Jun. 21, 2022, the complete disclosure of which is incorporated herein by reference for all purposes.

In some embodiments, various methods can use vagal nerve stimulation to suppress inflammation, thereby increasing the effectiveness of telomerase to maintain telomere lengths in cellular DNA. In some embodiments, some methods and devices involve the inhibition of pro-inflammatory cytokines, or more specifically, stimulation of the vagus nerve to inhibit and/or block the release of such pro-inflammatory cytokines. In some embodiments, some methods and devices use vagal nerve stimulation to increase the concentration or effectiveness of anti-inflammatory cytokines. A more complete description of these embodiments can be found in U.S. patent application Ser. No. 17/731,393, filed Apr. 28, 2022, the complete disclosure of which is incorporated herein by reference for all purposes.

The methods and devices disclosed herein can be used to prevent, diagnose, monitor, ameliorate, or treat a medical condition, a disease, or a disorder of a user, such as a mammal, such as an animal, such as a human, whether male or female, whether infant, child, adult, or elderly, or others.

For example, the devices can be configured to prevent, diagnose, monitor, ameliorate, or treat a neurological condition, such as epilepsy, headache, whether primary headaches, such as cluster, migraine or tension, or secondary headaches, caused by, for example, acute sinusitis, arterial tears, blood clots, aneurysms, glaucoma, tumors, medication overuse headaches, thunderclap headaches, concussion (e.g., post-concussion syndrome), trigeminal neuralgia and the like, seizures, vertigo, dizziness, aneurysm, palsy, Parkinson's disease, Alzheimer's disease, post-traumatic stress disorder (PTSD) or others, as understood to skilled artisans and which are only omitted here for brevity.

For example, the medical devices can be configured to prevent, diagnose, monitor, ameliorate, or treat conditions associated with replicating pathogens. The replicating pathogen may include a bacteria, fungi, protozoa, worm, infectious protein (e.g., prion) or a virus, such as an RNA virus. In one particular embodiment, the virus comprises a virus that contains a sensitizing and/or allergenic protein or other molecule that triggers an allergic or inflammatory response in the user, such as a virus in the coronaviridae or coronavirus family (e.g., COVID 19). The methods and systems of the present invention reduce the expression of inflammatory mediators that are elevated in ARDS and other inflammatory disorders, thereby ameliorating the overactivity of the immune reaction in user's suffering from certain disorders associated with replicating pathogen. This therapy provides potent anti-inflammatory activity without the negative side effect of conventional immune suppression techniques and drugs, such as steroids. In addition, the methods and systems of the present invention decrease the magnitude of constriction of bronchial smooth muscle, thereby improving the user's breathing in situations involving shortness of breath and impaired oxygen saturation, such as ARDS caused by certain replicating pathogens (e.g., COVID 19). A more complete description of these embodiments can be found in US Pat. No. 16,838,953, filed Apr. 2, 2020 and Ser. No. 17/472,962, filed Sep. 10, 2021, the complete disclosure of which is incorporated herein by reference for all purposes.

A more complete description of some of the applications for non-invasive nerve stimulation can be found in U.S. Pat. Nos. 9,037,247, 8,972,004, 8,868,177, 9,089,719, 8,874,205, 8,676,330, 10,279,163, 8,874,227, 9,174,066, 9,566,426, 9,174,045, 10,252,074, 9,126,050, 11,517,742, 10,232,178, 9,403,001, 9,174,049, 10,512,769, 11,432,760, 10,537,728, 11,590,341 and 11,027,127, the complete disclosures of which are incorporated herein by reference.

The fact that electrical stimulation of a vagus nerve can be used to treat many disorders and conditions may be understood as follows. The vagus nerve is composed of motor and sensory fibers. The vagus nerve leaves the cranium, passes down the neck within the carotid sheath to the root of the neck, then passes to the chest and abdomen, where it contributes to the innervation of the viscera. A human vagus nerve (tenth cranial nerve, paired left and right) comprises of over 100,000 nerve fibers (axons), mostly organized into groups. The groups are contained within fascicles of varying sizes, which branch and converge along the nerve. Under normal physiological conditions, each fiber conducts electrical impulses only in one direction, which is defined to be the orthodromic direction, and which is opposite the antidromic direction. However, external electrical stimulation of the nerve may produce action potentials that propagate in orthodromic and antidromic directions. Besides efferent output fibers that convey signals to the various organs in the body from the central nervous system, the vagus nerve conveys sensory (afferent) information about the state of the body's organs back to the central nervous system. Some 80-90% of the nerve fibers in the vagus nerve are afferent (sensory) nerves, communicating the state of the viscera to the central nervous system.

The largest nerve fibers within a left or right vagus nerve are approximately 20 μm in diameter and are heavily myelinated, whereas only the smallest nerve fibers of less than about 1 μm in diameter are completely unmyelinated. When the distal part of a nerve is electrically stimulated, a compound action potential may be recorded by an electrode located more proximally. A compound action potential contains several peaks or waves of activity that represent the summated response of multiple fibers having similar conduction velocities. The waves in a compound action potential represent different types of nerve fibers that are classified into corresponding functional categories, with approximate diameters as follows: A-alpha fibers (afferent or efferent fibers, 12-20 μm diameter), A-beta fibers (afferent or efferent fibers, 5-12 μm), A-gamma fibers (efferent fibers, 3-7 μm), A-delta fibers (afferent fibers, 2-5 μm), B fibers (1-3 μm) and C fibers (unmyelinated, 0.4-1.2 μm). The diameters of group A and group B fibers include the thickness of the myelin sheaths.

The vagus (or vagal) afferent nerve fibers arise from cell bodies located in the vagal sensory ganglia, which take the form of swellings near the base of the skull. Vagal afferents traverse the brainstem in the solitary tract, with some eighty percent of the terminating synapses being located in the nucleus of the tractus solitarius (or nucleus tractus solitarii, nucleus tractus solitarius, or NTS). The NTS projects to a wide variety of structures in the central nervous system, such as the amygdala, raphe nuclei, periaqueductal gray, nucleus paragigantocellurlais, olfactory tubercule, locus ceruleus, nucleus ambiguous and the hypothalamus. The NTS also projects to the parabrachial nucleus, which in turn projects to the hypothalamus, the thalamus, the amygdala, the anterior insula, and infralimbic cortex, lateral prefrontal cortex, and other cortical regions [JEAN A. The nucleus tractus solitarius: neuroanatomic, neurochemical and functional aspects. Arch Int Physiol Biochim Biophys 99(5,1991): A3-A52 the disclosure of which is incorporated herein by reference for all purposes as if copied and pasted herein]. Thus, stimulation of vagal afferents can modulate the activity of many structures of the brain and brainstem through these projections.

With regard to vagal efferent nerve fibers, two vagal components have evolved in the brainstem to regulate peripheral parasympathetic functions. The dorsal vagal complex, consisting of the dorsal motor nucleus and its connections controls parasympathetic function primarily below the level of the diaphragm, while the ventral vagal complex, comprised of nucleus ambiguous and nucleus retrofacial, controls functions primarily above the diaphragm in organs such as the heart, thymus and lungs, as well as other glands and tissues of the neck and upper chest, and specialized muscles such as those of the esophageal complex. For example, the cell bodies for the preganglionic parasympathetic vagal neurons that innervate the heart reside in the nucleus ambiguus, which is relevant to potential cardiovascular side effects that may be produced by vagus nerve stimulation.

The vagus efferent fibers innervate parasympathetic ganglionic neurons that are located in or adjacent to each target organ. The vagal parasympathetic tone resulting from the activity of these fibers is balanced reflexively in part by sympathetic innervations. Consequently, electrical stimulation of a vagus nerve may result not only in modulation of parasympathetic activity in postganglionic nerve fibers, but also a reflex modulation of sympathetic activity. The ability of a vagus nerve to bring about widespread changes in autonomic activity, either directly through modulation of vagal efferent nerves, or indirectly via activation of brainstem and brain functions that are brought about by electrical stimulation of vagal afferent nerves, accounts for the fact that vagus nerve stimulation can treat many different medical conditions in many end organs. Selective treatment of particular conditions is possible because the parameters of the electrical stimulation (e.g. frequency, amplitude, pulse width, etc.) may selectively activate or modulate the activity of particular afferent or efferent A, B, and/or C fibers that result in a particular physiological response in each individual.

### Treatment Paradigms

Applicant has discovered that it is not necessary to “continuously stimulate” the vagus nerve or to in order to provide efficacious benefits to users. The term “continuously stimulate” as defined herein means stimulation that follows a certain On/Off pattern continuously 24 hours/day. For example, existing implantable vagal nerve stimulators “continuously stimulate” the vagus nerve with a pattern of 30 seconds ON/5 minutes OFF (or the like) for 24 hours/day and seven days/week. Applicant has determined that this continuous stimulation is not necessary to provide the desired benefits of the device.

A vagus nerve stimulation treatment is conducted prior to sleep for a continuous period of thirty seconds to five minutes, preferably about 90 seconds to about three minutes and more preferably about two minutes (each defined as a single dose). After a dose has been completed, the therapy is stopped for a period of time (depending on the treatment as described below).

In embodiments, the treatment paradigm is sufficient to alter a microglia in a central nervous system of the user from a substantially pro-inflammatory state to a substantially non-inflammatory state. The treatment paradigm may be sufficient to reduce astrocytic activation with the central nervous system of the user. The treatment paradigm may be sufficient to increase glymphatic clearance of waste products within the brain of the user. The waste products comprise beta-amyloid, tau proteins and oxidative byproducts.

In one embodiment, the treatment paradigm comprises delivering the electrical impulses for at least 30 seconds within 4 hours of a commencement of sleep by the user, or for about 30 seconds to about 5 minutes within 3 hours, or 2 hours or 1 hour prior to commencement of sleep. The electrical impulse may be applied in a single dose for a time period of about 30 seconds and about 5 minutes, preferably about 90-150 seconds, or it may be applied in a series of doses each having a time period of about 30 seconds to about 3 minutes, preferably about 90-150 seconds in each dose. The series of doses may be applied every 5 to 30 minutes, or every 10 to 20 minutes, or every 15 minutes, for a period of at least 1 hour, or at least 2 hours or about 3 hours.

For certain treatments, the time of day can be more important than the time interval between treatments. For example, the locus correleus has periods of time during a 24 hour day wherein it has inactive periods and active periods. Typically, the inactive periods can occur in the late afternoon or in the middle of the night when the user is asleep. It is during the inactive periods that the levels of inhibitory neurotransmitters in the brain that are generated by the locus correleus are reduced. This may have an impact on certain applications.

In these embodiments, the prophylactic treatment may comprise multiple doses/day timed for periods of inactivity of the locus correleus. In one embodiment, a treatment comprises one or more doses administered 2-3 times per day or 2-3 “treatment sessions” per day. The treatment sessions preferably occur during the late afternoon or late evening, in the middle of the night and again in the morning when the user wakes up. In an exemplary embodiment, each treatment session comprises 1-4 doses, preferably 2-3 doses, with each dose lasting for about 60 seconds to about 5 minutes, preferably about 90 seconds to about three minutes.

For other applications, the intervals between treatment sessions may be the most important as applicant has determined that stimulation of the vagus nerve can have a prolonged effect on the inhibitor neurotransmitters levels in the brain, e.g., at least one hour, up to 3 hours and sometimes up to 8 hours. In one embodiment, a treatment comprises one or more doses (i.e., treatment sessions) administered at intervals during a 24 hour period. In a preferred embodiment, there are 1-5 such treatment sessions, preferably 2-4 treatment sessions. Each treatment session preferably comprises 1-3 doses, each “dose” lasting between about 60 seconds to about five minutes, preferably about 90 seconds to about 150 seconds, more preferably about 2 minutes.

For all of the treatments listed above, one may alternate treatment between left and right sides, or one may treat ipsilateral or contralateral to one side of the brain, respectively. Or for a single treatment, one may treat one minute on one side followed by one minute on the opposite side. Variations of these treatment paradigms may be chosen on a user-by-user basis. However, it is understood that parameters of the stimulation protocol may be varied in response to heterogeneity in the symptoms of users. Different stimulation parameters may also be selected as the course of the user's condition changes. In preferred embodiments, the disclosed methods and devices do not produce clinically significant side effects, such as agitation or anxiety, or changes in heart rate or blood pressure. A more complete description of some of these embodiments can be found in U.S. Pat. No. 10,441,780, the complete disclosure of which is incorporated herein by reference.

### Description Of Various Nerve Stimulating/Modulating Devices

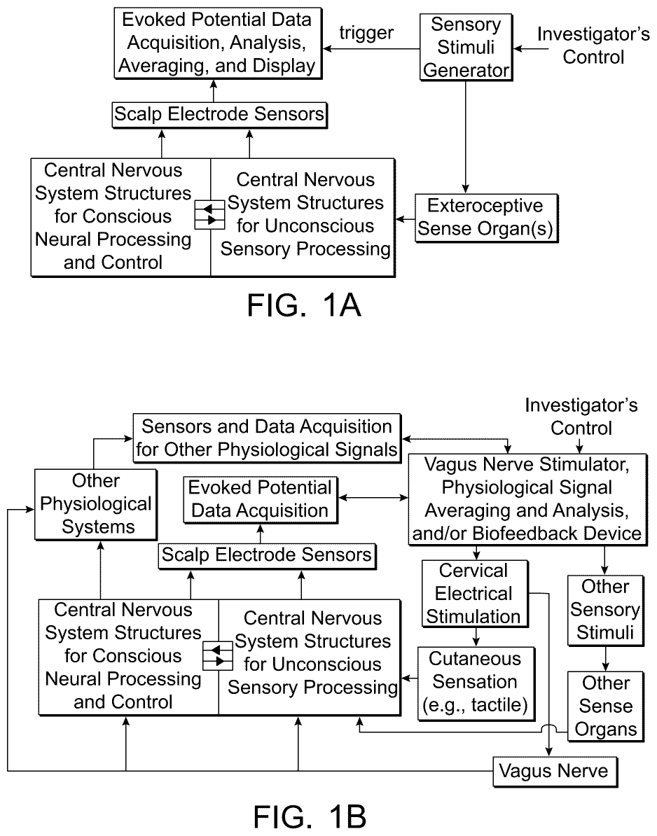

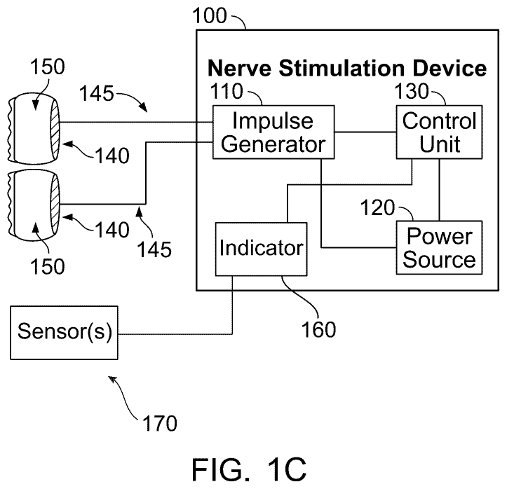

Referring now to FIG. 1, an electrode-based nerve stimulating and/or modulating device 100 is provided for delivering impulses of energy to nerves. As shown, device 100 may include an impulse or signal generator 110, an energy or power source 120 coupled to the impulse generator 110 and/or a control unit 130 in communication with the impulse generator 110 and coupled to the energy source 120. Device 100 further includes one or more electrodes 140 coupled via wires 145 (or wirelessly) to impulse generator 110. Alternatively, electrodes 140 may be housed within, or on the outer surface of, device 100.

In some embodiments, the same impulse generator 110, energy source 120, and control unit 130 may be used for either a magnetic stimulator or an electrode-based stimulator, allowing the user to change parameter settings depending on whether magnetic coils or the electrodes 140 are attached.

Although a pair of electrodes 140 is shown in FIG. 1, in practice the electrodes may also comprise one electrode, or three or more distinct electrode elements, each of which is connected in series or in parallel to the impulse generator 110. Thus, the electrodes 140 that are shown in FIG. 1 represent some, most, many, or all electrodes of the device collectively.

Electrodes 140 may include a suitable adhesive that secured them to a skin surface. Suitable adhesive electrodes for use herein may include electrode pads, self-adhesive electrodes or the like. In this embodiment, electrodes 140 may be placed in a suitable location on the user's neck and adhered thereto. Electrodes 140 receive electrical impulses from pulse generator 110. The duration, amplitude, frequency and treatment paradigm for the electrical impulses may be controlled by controller 130, a mobile device, a remote computer, processor or server, or via another electronic device coupled to pulse generator 110. Suitable mobile devices include a wearable computing devices, such as a smartwatch, Whoop®, Fitbit®, Garmin® or the like, a mobile phone, a mobile processing device (e.g., laptop computers or tablets) and the like. This embodiment allows, for example, a physician to secure electrodes 140 to the user's neck such that the treatment paradigm may be followed without user involvement. This is particularly useful for treating users that are unable or unwilling to self-treat. For example, in some cases, users recovering from surgery, such as major colorectal surgery may be either incapable of self-treatment, or their compliance with the treatment protocol may not be complete. In another example, older users may not have suitable mental faculties for self-treatment.

Stimulator 100, or certain components of stimulator, may be housed in an outer housing, or a covering or patch 330 to protect stimulator from the environment. The patch may include a suitable adhesive strip or pad on one surface for adhering the patch and stimulator to the outer skin surface of the user.

The stimulator in this embodiment includes one or more electrodes. The stimulator may also include a power source such as a battery, and a signal generator for applying the electrical impulses to the electrodes. In one such embodiment, the power source, e.g., a battery, is disposed within the patch and coupled to the electrodes. In another embodiment, the signal generator includes flexible circuitry within the patch and coupled to the energy source and the electrodes. Alternatively, the power source and/or the signal generator may be remote from the patch and wirelessly coupled, or directly connected to the electrodes, as discussed above. An external controller may be wirelessly coupled to the stimulator to provide a stimulation protocol to the signal generator and to control other key functions of the signal, such as power, amplitude, duration frequency and the like.

The stimulator may reside in a housing that is removably coupled to the patch via a snap-fitting, Velcro, or other suitable attachment means. In this embodiment, the patch may be adhered to the user and the stimulator may be removed and reattached without removing the patch. This allows the healthcare professions to, for example, recharge the battery, troubleshoot the device and/or control the stimulation therapy on the device.

The stimulator may also include a conductive fluid, such as a gel pad, disposed between the electrode(s) and the user's outer skin surface to enhance conductivity of the electrical impulses through the outer skin surface to the nerve.

Alternatively, the outer covering may comprise any wearable material that may include the stimulator. For example, depending on the location of the target nerve on the user's body, the stimulator may be attached to, or embedded within, a wearable garment, such as a shirt, scarf, watch, hat, gloves, pants, shoes, boots, socks, underwear, belt, dress, jacket, sweater, ear muffs, or the like. The wearable garment may also comprise an accessory, such as a wristband, ankle or wrist bracelet, necklace, earrings, a compression garment, an ankle or knee brace or the like.

In yet another embodiment, the garment itself is the stimulator. For example, the garment may comprise an electronic textile or e-textile that includes fabrics that enable digital components, such as electrodes, pulse generators, batteries wireless receivers and other electronic components to be embedded therein. Electronic textiles are distinct from wearable garments because the emphasis is placed on the seamless integration of textiles with electronic elements like microcontrollers, sensors, and actuators. In one embodiment, the electronic textile may comprise an organic electronics material that is conducting and has insulated electrical components that allows the garment to be washed without damaging the electronic components.

The stimulator may also include an array of electrodes. The electrode array may include multiple sets of electrodes with each set of electrodes configured to apply electrical impulses through the outer skin surface of the user, as discussed above. Each of the sets of electrodes may be individually coupled to the pulse generator, either directly, through wires, or wireless as described above. The electrode array may have multiple patterns. For example, the array may be linear, square, circular or any other suitable shape.

In certain embodiments, the electrode array comprises two or more sets of electrodes, each spaced apart from each other between about 2 mm to about 25 mm, preferably between about 4 mm to about 10 mm. The electrode array preferably comprises a shape that substantially corresponds to a target area of the user's neck. In one embodiment, the target area is the area on the neck that allows for electrical impulses to be passed through the skin to the vagus nerve (discussed in detail below).

The electrode sets may each be individually coupled to the pulse generator and/or the controller such that electrical impulses can be applied to all of the electrode sets, some of the electrode sets or only one of the electrode sets. In certain embodiments, the controller is configured to apply electrode impulses to only those electrodes positioned optimally for stimulating the nerve. In addition, the selection of electrodes may be dynamic and change over time.

In one such embodiment, the electrodes are arranged in an array or matrix that may contain tens to hundreds of microelectrodes. The microelectrodes may each be independently coupled to the pulse generator 110 such that the pulse generator can apply current to any one or a plurality of the microelectrodes. In some embodiments, groups of the microelectrodes are coupled together and then coupled to the pulse generator 110 such that electric current can be applied independently to each group. In an exemplary embodiment, the electrodes have a size of about 0.5 to 2.0 mm, preferably about 1.0 mm, and are spaced from each other a distance of about 0.5 to about 10 mm, preferably between about 2.0 mm and 5.0 mm (e.g., 3.0 mm).

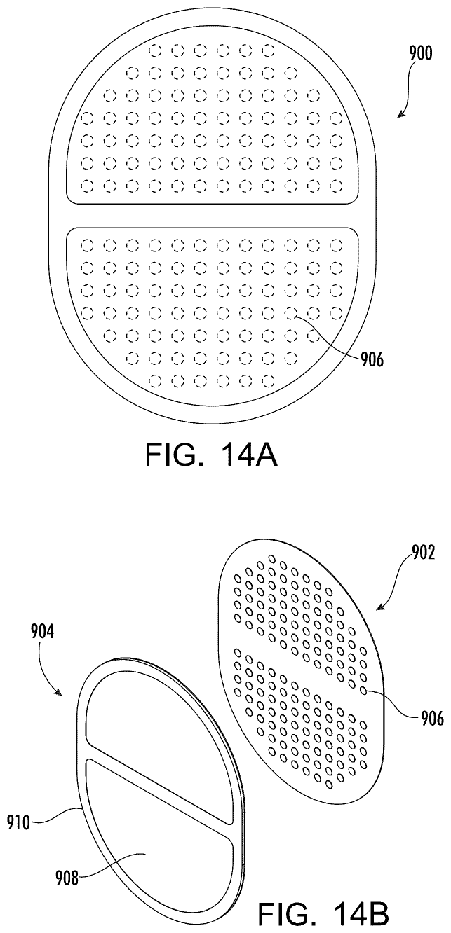

FIGS. 14A and 14B illustrate one example of an electrode array 900 that includes a flexible PCB board 902 and a cover 904. The PCB board 902 may comprise any number of microelectrodes 906 that are coupled to pulse generator 110 as described above. In the representative embodiment, the PCB board 902 includes about 20 to about 1,000 microelectrodes 906, or between about 40 to about 200 microelectrodes 906. In certain embodiments, cover 904 may include a conductive surface 908 overlying electrodes 906 and a non-conductive surface 910 overlying the remaining portions of the PCB board 902. Conductive surface 908 may include a conductive gel (not shown) and non-conductive surface may include a non-conductive gel.

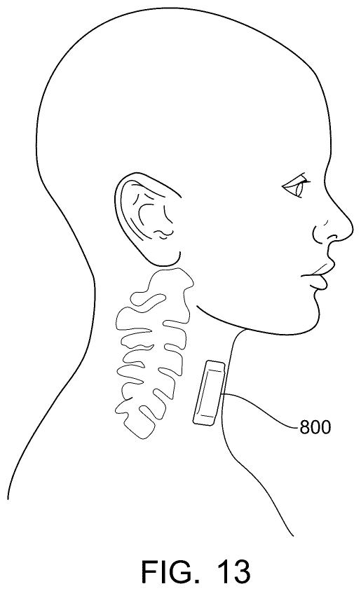

Electrode array 900 may be included on the housing of a stimulator device, such as those described below. Alternatively, array 900 may be included as part of a patch, such as the patch 800 shown in FIG. 13 and discussed below. The dimensions of PCB board 902 will largely depend on the dimensions of the target region of the skin surface on the patient. In certain embodiments, the dimensions will be selected to encompass a region of the outer skin surface of the neck that is near the carotid sheath of the patient.

Stimulator 100 further comprises one or more sensors 170 coupled to stimulator 100 and/or electrodes 140 (or the microelectrodes in the array) for detecting whether the nerve has been stimulated, the amplitude of the stimulation, or whether the nerve has been stimulated with sufficient amplitude and other parameters to fire an action potential. The sensors 170 may detect a physiological parameter of the user. Physiological parameters may include, for example, blood flow associated with a nerve, such as vagal artery or cerebral blood flow, heart rate or variability, ECG, respiration status, depth and rate, core temperature, hydration, blood pressure, brain function, oxygenation, skin impedance, and skin temperature, pupil diameter (e.g., pupil dilation), galvanic skin response, selected biomarkers or other chemicals, a property of a voice of the user, a laryngeal electromyographic signal, an electroglottographic signal, a property of the autonomic nervous system and the like. Alternatively, the sensors 170 may be coupled to the electrodes 140 and may sense one or more parameters of the electrodes, such as impedance, amplitude, voltage or the like.

The sensors 170 may also be coupled to the controller 130. In this embodiment, the controller 130 is configured to receive input from the sensors and to direct the pulse generator 110 to apply electrical impulses to one or more sets of the electrodes 140 based on this input. For example, the sensors 170 may provide data that suggests that one or more of the sets of electrodes is not positioned properly to stimulate the nerve, or to stimulate the nerve at the optimal signal strength to cause the nerve to fire an action potential. The controller 130 is configured to shift the electrical impulse to the set or sets of electrodes that provide a sufficient electrical impulse to the nerve to cause it to fire an action potential. In this manner, the controller 130 can optimize the application of the electrical impulses to the nerve.

Sensor(s) 170 may be coupled to electrodes 140, or they may be formed as part of the electrodes 140. Alternatively, sensor(s) 140 may be only coupled to stimulator 100, or they may be coupled to a separate device, such as a mobile device (discussed below). In certain embodiments, stimulator 100 will comprise a housing that includes both electrodes 140 and sensors 170, as discussed in more detail below.

In certain embodiments, sensor(s) 170 are configured to detect a target position for stimulating a selected nerve within a user. The target position may, for example, be located on an outer skin surface of the user and the selected nerve may be located within the user under the skin surface. In some cases, the selected nerve may be located deep within the user, i.e., greater than about 5 mm below the outer skin surface, greater than about 10 mm, or even greater than about 20 mm below. In one such embodiment, the selected nerve is the vagus nerve and the target location is a position on the outer skin surface of the neck and/or the ear of the user suitable for passing an electrical impulse through the skin sufficient to modulate the vagus nerve.

In one embodiment, sensor 170 comprises a heart pulse sensor configured to detect a heart pulse in the user. The heart pulse sensor may be any suitable sensor known in the art for detecting the heart pulse of a user, such as an infrared sensor, optical sensor, tactile sensor, a photoplethysmography (PPG) sensor or the like. The heart pulse sensor may, for example, detect the change in volume of a blood vessel that occurs when the heart pumps blood. Alternatively, the heart pulse sensor may detect vibrations, sounds or other indications that the sensor 170 is located adjacent to, or near, the user's heart pulse.

The heart pulse sensor is preferably designed to contact the user's outer skin surface and detect a pulse adjacent to, or near the sensor. However, in certain embodiments, the heart pulse sensor may be designed to detect the heart pulse without contacting the skin surface, e.g., through vibration, sound or other detection mechanisms. In these embodiments, sensor 170 may, for example, be located within stimulator 100, or within a separate device.

Sensors 170 may be coupled to an indicator 160 within stimulator 100, or within a separate device, such as a mobile device (discussed in more detail below). Indicator 160 is configured to generate an alert when sensors 170 have detected the target nerve. The alert may be, for example, a visual, tactile or audial alert, that provides the user with an indication that the sensor 170 has detected the target location.

In one such embodiment, sensor 170 comprises a heart pulse sensor that is configured to detect a heart pulse emanating from a blood vessel in the user, such as the carotid artery in the user's neck, the temporal artery at the temple above and to the outer side of the eye, the radial artery in the user's wrist, the elbow of the top of the foot. In an exemplary embodiment, the heart pulse sensor is configured to detect a heart pulse in the carotid artery. The vagus nerve is situated within the carotid sheath, near the carotid artery and the interior jugular vein. The carotid sheath is located at the lateral boundary of the retropharyngeal space on each side of the neck and deep to the sternocleidomastoid muscle. A more complete description of some of these embodiments can be found in U.S. patent application Ser. No. 17/744,557, filed May 13, 2022, the complete disclosure of which is incorporated herein by reference for all purposes.

The three major structures within the carotid sheath are the common carotid artery, the internal jugular vein and the vagus nerve. The carotid artery lies medial to the internal jugular vein, and the vagus nerve is situated posteriorly between the two vessels. Proceeding from the skin of the neck above the sternocleidomastoid muscle to the vagus nerve, a line may pass successively through the sternocleidomastoid muscle, the carotid sheath and the internal jugular vein, unless the position on the skin is immediately to either side of the external jugular vein. In the latter case, the line may pass successively through only the sternocleidomastoid muscle and the carotid sheath before encountering the vagus nerve, missing the interior jugular vein. Accordingly, a point on the neck adjacent to the external jugular vein might be preferred for non-invasive stimulation of the vagus nerve.

Sensors 170 are configured to detect the heart pulse emanating from the carotid artery to provide an indication that electrodes 150 are located adjacent to, or near the carotid sheath and/or the external jugular vein and thus near the vagus nerve. This provides confirmation to the user that the device is positioned optimally for stimulating the vagus nerve.

In certain embodiments, sensors 170 may be configured to detect a magnitude of the heart pulse emanating from the carotid artery. In these embodiments, the sensors 170 may be configured, for example, to only provide an indication that the heart pulse has been detected when the magnitude of heart pulse reaches a threshold level, indicating that the sensor is close to the carotid artery. Alternatively, the sensors 170 may transmit the magnitude of heart pulse detected to a controller or suitable electronics within stimulator, or a separate mobile device.

In certain embodiments, indicator 160 is configured to transmit an alert that is associated with the magnitude of the heart pulse. For example, the alert may comprise an audible sound that increases in decibel level as the magnitude increase. In another example, the alert may comprise a vibration that increases in intensity or frequency as the magnitude of the heart pulse increases. In yet another example, the alert may comprise a visual signal, such as a blinking light that increases in intensity with heart pulse magnitude, different colored lights associated with threshold magnitudes of heart pulse, or another visual signal, such as bars, lines or other shapes that increase in size (e.g., length or width) with increasing heart pulse magnitude.

The indicator 160 may further be configured to provide a second alert when the magnitude of the heart pulse reaches a threshold level associated with optimal positioning of the sensor 170 and/or the electrodes 150. For example, if the indicator is providing a blinking light that increases in intensity with heart pulse magnitude, the second alert may be that the blinking light stops blinking and becomes constant, or it changes color (e.g., from yellow to green), or a separate alert is produced, such as a sound, vibration or the like.

In this embodiment, the sensor 160 may comprise a heart pulse sensor configured to contact the outer skin surface of the user and directly detect the pulse within the carotid sheath, such as an infrared sensor, optical sensor, tactile sensor, a photoplethysmography (PPG) sensor or the like.

Alternatively, the sensor 160 may comprise an ultrasound transducer or probe configured to detect the location of the vagus nerve underlying stimulator 100. The probe may be housed within stimulator 100, or it may be a separate device. The probe may be connected to an ultrasound machine that displays the anatomical structures that lie under the probe. Alternatively, the probe may be coupled to a controller or other device that is configured to provide an indication or alert when the probe has illustrated the carotid sheath.

The control unit 130 controls the impulse generator 110 to generate a signal for each of the device's electrodes (or magnetic coils). The signals are selected to be suitable for amelioration of a particular medical condition when the signals are applied non-invasively to a target nerve or tissue via the electrodes 140. It is noted that nerve stimulating/modulating device 100 may be referred to by its function as a pulse generator. Patent application publications US2005/0075701 and US2005/0075702, both to SHAFER, the disclosure of which is incorporated herein by reference for all purposes as if copied and pasted herein, contain descriptions of pulse generators that may be applicable to this disclosure. By way of example, a pulse generator is also commercially available, such as Agilent 33522A Function/Arbitrary Waveform Generator, Agilent Technologies, Inc., 5301 Stevens Creek Blvd Santa Clara CA 95051.

The control unit 130 may comprise a general purpose computer, comprising one or more CPU, computer memories for the storage of executable computer programs (including the system's operating system) and the storage and retrieval of data, disk storage devices, communication devices (such as serial and USB ports) for accepting external signals from a keyboard, computer mouse, and touchscreen, as well as any externally supplied physiological signals, analog-to-digital converters for digitizing externally supplied analog signals, communication devices for the transmission and receipt of data to and from external devices such as printers and modems that comprise part of the system, hardware for generating the display of information on monitors or display screens that comprise part of the system, and busses to interconnect the above-mentioned components. Thus, the user may operate the system by typing or otherwise providing instructions for the control unit 130 at a device such as a keyboard or touchscreen and view the results on a device such as the system's computer monitor or display screen, or direct the results to a printer, modem, and/or storage disk. Control of the system may be based upon feedback measured from externally supplied physiological or environmental signals. Alternatively, the control unit 130 may have a compact and simple structure, for example, wherein the user may operate the system using only an on/off switch and energy control wheel or knob, or their touchscreen equivalent. In a section below, an embodiment is also described wherein the stimulator housing has a simple structure, but other components of the control unit 130 are distributed into other devices.

Parameters for the nerve or tissue stimulation include energy level, frequency and train duration (or pulse number). The stimulation characteristics of each pulse, such as depth of penetration, strength and selectivity, depend on the rise time and peak electrical energy transferred to the electrodes, as well as the spatial distribution of the electric field that is produced by the electrodes. The rise time and peak energy are governed by the electrical characteristics of the stimulator and electrodes, as well as by the anatomy of the region of current flow within the user. In some embodiments, pulse parameters are set in such a way as to account for the detailed anatomy surrounding the nerve that is being stimulated [Bartosz SAWICKI, Robert Szmurlo, Przemyslaw Plonecki, Jacek Starzynski, Stanislaw Wincenciak, Andrzej Rysz. Mathematical Modelling of Vagus Nerve Stimulation. pp. 92-97 in: Krawczyk, A. Electromagnetic Field, Health and Environment: Proceedings of EHE'07. Amsterdam, IOS Press, 2008, the disclosure of which is incorporated herein by reference for all purposes as if copied and pasted herein]. Pulses may be monophasic, biphasic or polyphasic. In some embodiments, some devices include those that are fixed frequency, where each pulse in a train has the same inter-stimulus interval, and those that have modulated frequency, where the intervals between each pulse in a train can be varied.

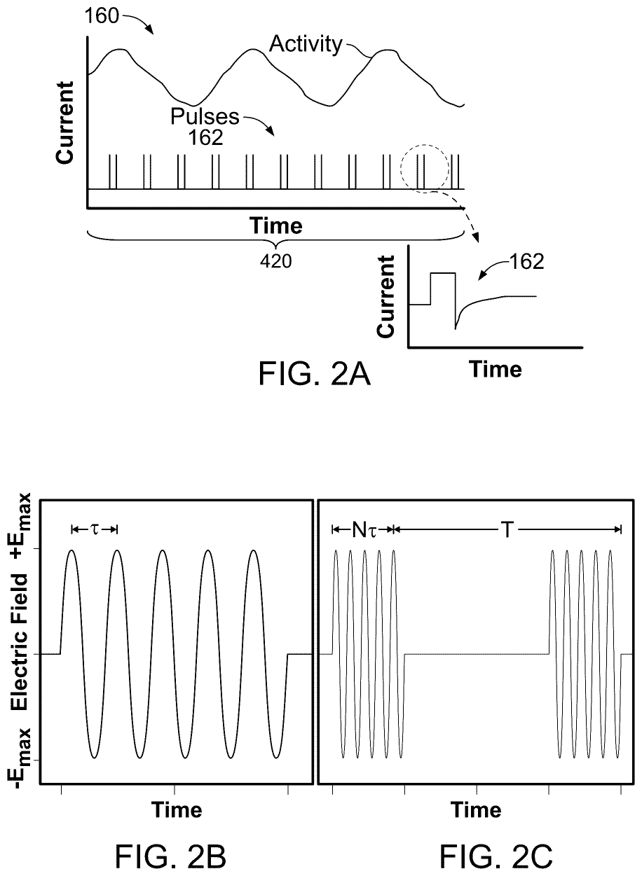

FIG. 2A illustrates an example of an electrical voltage/current profile for a stimulating, blocking and/or modulating impulse applied to a portion or portions of selected nerves in accordance with an embodiment of this disclosure. For some embodiments, the voltage and current refer to those that are non-invasively produced within the user by the electrodes (or magnetic coils). As shown, a suitable electrical voltage/current profile 160 for the blocking and/or modulating impulse 162 to the portion or portions of a nerve may be achieved using pulse generator 110. In some embodiments, the pulse generator 100 may be implemented using an energy source 120 and a control unit 130 having, for instance, a processor, a clock, a memory, etc., to produce a pulse train 164 to the electrodes 140 that deliver the stimulating, blocking and/or modulating impulse 162 to the nerve. Nerve stimulating/modulating device 100 may be externally energized and/or recharged or may have its own energy source 120. The parameters of the modulation signal 160, such as the frequency, amplitude, duty cycle, pulse width, pulse shape, etc., can be programmable, non-programmable, modifiable, locally or remotely updateable, or others. An external communication device may modify the pulse generator programming to improve treatment.

In addition, or as an alternative to some of the devices to implement the modulation unit for producing the electrical voltage/current profile of the stimulating, blocking and/or modulating impulse to the electrodes, the device disclosed in US Patent Application Publication No. US2005/0216062, the disclosure of which is incorporated herein by reference for all purposes as if copied and pasted herein, may be employed. That patent publication discloses a multifunctional electrical stimulation (ES) system adapted to yield output signals for effecting electromagnetic or other forms of electrical stimulation for a broad spectrum of different biological and biomedical applications, which produce an electric field pulse in order to non-invasively stimulate nerves. The system includes an ES signal stage having a selector coupled to a plurality of different signal generators, each producing a signal having a distinct shape, such as a sine wave, a square or a saw-tooth wave, or simple or complex pulse, the parameters of which are adjustable in regard to amplitude, duration, repetition rate and other variables. Examples of the signals that may be generated by such a system are described in a publication by LIBOFF [A. R. LIBOFF. Signal shapes in electromagnetic therapies: a primer. pp. 17-37 in: Bioelectromagnetic Medicine (Paul J. Rosch and Marko S. Markov, eds.). New York: Marcel Dekker (2004), the disclosure of which is incorporated herein by reference for all purposes as if copied and pasted herein]. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated, as well as the outputs of various sensors which sense prevailing conditions prevailing in this substance, whereby the user of the system can manually adjust the signal, or have it automatically adjusted by feedback, to provide an electrical stimulation signal of whatever type the user wishes, who can then observe the effect of this signal on a substance being treated.

The stimulating and/or modulating impulse signal 160 preferably has a frequency, an amplitude, a duty cycle, a pulse width, a pulse shape, etc. selected to influence the therapeutic result, namely, stimulating and/or modulating some or all of the transmission of the selected nerve. For example, the frequency may be about 1 Hz or greater, such as between about 15 Hz to 100 Hz, preferably between about 15-50 Hz and more preferably between about 15-35 Hz. In some embodiments, the frequency is 25 Hz. The modulation signal may have a pulse width selected to influence the therapeutic result, such as about 1 microseconds to about 1000 microseconds, preferably about 100-400 microseconds and more preferably about 200-400 microseconds. For example, the electric field induced or produced by the device within tissue in the vicinity of a nerve may be about 5 to 600 V/m, preferably less than 100 V/m, and even more preferably less than 30 V/m. The gradient of the electric field may be greater than 2 V/m/mm. More generally, the stimulation device produces an electric field in the vicinity of the nerve that is sufficient to cause the nerve to depolarize and reach a threshold for action potential propagation, which is approximately 8 V/m at 1000 Hz. The modulation signal may have a peak voltage amplitude selected to influence the therapeutic result, such as about 0.2 volts or greater, such as about 0.2 volts to about 40 volts, preferably between about 1-20 volts and more preferably between about 2-12 volts.

In an exemplary embodiment, the waveform comprises bursts of sinusoidal pulses, as shown in FIGS. 2B and 2C. As seen there, individual sinusoidal pulses have a period oft, and a burst consists of N such pulses. This is followed by a period with no signal (the inter-burst period). The pattern of a burst followed by silent inter-burst period repeats itself with a period of T. For example, the sinusoidal period τ may be between about 50-1000 microseconds with a frequency of about 1-20 kHz), preferably between about 100-400 microseconds with a frequency of about 2.5-10 kHz, more preferably about 133-400 microseconds with a frequency of about 2.5-7.5 kHz and even more preferably about 200 microseconds with a frequency of about 5 kHz; the number of pulses per burst may be N=1-20, preferably about 2-10 and more preferably about 5; and the whole pattern of burst followed by silent inter-burst period may have a period T comparable to about 5-100 Hz, preferably about 15-50 Hz, more preferably about 25-35 Hz and even more preferably about 25 Hz (a much smaller value of T is shown in FIG. 2E to make the bursts discernable). When these exemplary values are used for T and τ, the waveform contains significant Fourier components at higher frequencies ( 1/200 microseconds=5000/sec), as compared with those contained in transcutaneous nerve stimulation waveforms, as currently practiced.

The above waveform is essentially a 1-20 kHz signal that includes bursts of pulses with each burst having a frequency of about 5-100 Hz and each pulse having a frequency of about 1-20 kHz. Another way of thinking about the waveform is that it is a 1-20 kHz waveform that repeats itself at a frequency of about 5-100 Hz.

Invasive nerve stimulation typically uses square wave pulse signals. However, Applicant found that square waveforms are not ideal for non-invasive stimulation, as they produce excessive pain, but still can be used. Prepulses and similar waveform modifications have been suggested as methods to improve selectivity of nerve stimulation waveforms, but Applicant also did not find them ideal, although they still can be used [Aleksandra VUCKOVIC, Marco Tosato and Johannes J Struijk. A comparative study of three techniques for diameter selective fiber activation in the vagal nerve: anodal block, depolarizing prepulses and slowly rising pulses. J. Neural Eng. 5 (2008): 275-286, the disclosure of which is incorporated herein by reference for all purposes as if copied and pasted herein; Aleksandra VUCKOVIC, Nico J. M. Rijkhoff, and Johannes J. Struijk. Different Pulse Shapes to Obtain Small Fiber Selective Activation by Anodal Blocking—A Simulation Study. IEEE Transactions on Biomedical Engineering 51(5,2004):698-706, the disclosure of which is incorporated herein by reference for all purposes as if copied and pasted herein; Kristian HENNINGS. Selective Electrical Stimulation of Peripheral Nerve Fibers: Accommodation Based Methods. Ph.D. Thesis, Center for Sensory-Motor Interaction, Aalborg University, Aalborg, Denmark, 2004, the disclosure of which is incorporated herein by reference for all purposes as if copied and pasted herein].

In some embodiments, the use of feedback to generate the modulation signal 160 may result in a signal that is not periodic, particularly if the feedback is produced from sensors that measure naturally occurring, time-varying aperiodic physiological signals from the user. In fact, the absence of significant fluctuation in naturally occurring physiological signals from a user is ordinarily considered to be an indication that the user is in ill health. This is because a pathological control system that regulates the user's physiological variables may have become trapped around only one of two or more possible steady states and is therefore unable to respond normally to external and internal stresses. Accordingly, even if feedback is not used to generate the modulation signal 160, it may be useful to artificially modulate the signal in an aperiodic fashion, in such a way as to simulate fluctuations that would occur naturally in a healthy individual. Thus, the noisy modulation of the stimulation signal may cause a pathological physiological control system to be reset or undergo a non-linear phase transition, through a mechanism known as stochastic resonance [B. SUKI, A. Alencar, M. K. Sujeer, K. R. Lutchen, J. J. Collins, J. S. Andrade, E. P. Ingenito, S. Zapperi, H. E. Stanley, Life-support system benefits from noise, Nature 393 (1998) 127-128, the disclosure of which is incorporated herein by reference for all purposes as if copied and pasted herein; W Alan C MUTCH, M Ruth Graham, Linda G Girling and John F Brewster. Fractal ventilation enhances respiratory sinus arrhythmia. Respiratory Research 2005, 6:41, pp. 1-9, the disclosure of which is incorporated herein by reference for all purposes as if copied and pasted herein].

In some embodiments, the modulation signal 160, with or without feedback, will stimulate the selected nerve fibers in such a way that one or more of the stimulation parameters (e.g., energy, frequency, and others mentioned herein) are varied by sampling a statistical distribution having a mean corresponding to a selected, or to a most recent running-averaged value of the parameter, and then setting the value of the parameter to the randomly sampled value. The sampled statistical distributions will comprise Gaussian and 1/f, obtained from recorded naturally occurring random time series or by calculated formula. Parameter values will be so changed periodically, or at time intervals that are themselves selected randomly by sampling another statistical distribution, having a selected mean and coefficient of variation, where the sampled distributions comprise Gaussian and exponential, obtained from recorded naturally occurring random time series or by calculated formula.

In some embodiments, some devices, as disclosed herein, are provided in a “pacemaker” type form, in which electrical impulses 162 are generated to a selected region of the nerve by a stimulator device on an intermittent basis, to create in the user a lower reactivity of the nerve.

### Embodiments Of The Nerve Stimulators

The electrodes of the some of the devices, as disclosed herein, are applied to the surface of the neck, or to some other surface of the body, and are used to deliver electrical energy non-invasively to a nerve. Embodiments may differ with regard to the number of electrodes that are used, the distance between electrodes, and whether disk, ring or other shapes of electrodes are used. In some embodiments, one selects the electrode configuration for individual users, in such a way as to optimally focus electric fields and currents onto the selected nerve, without generating excessive currents on the surface of the skin.

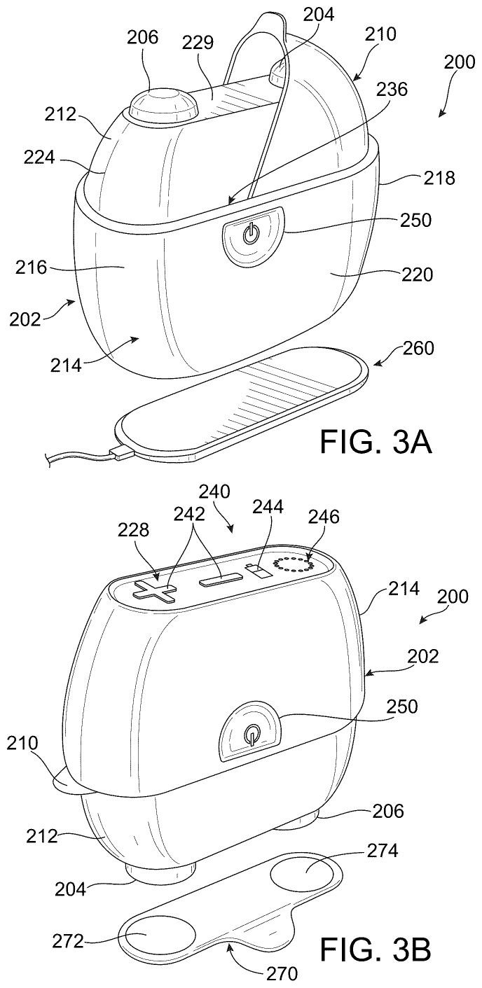

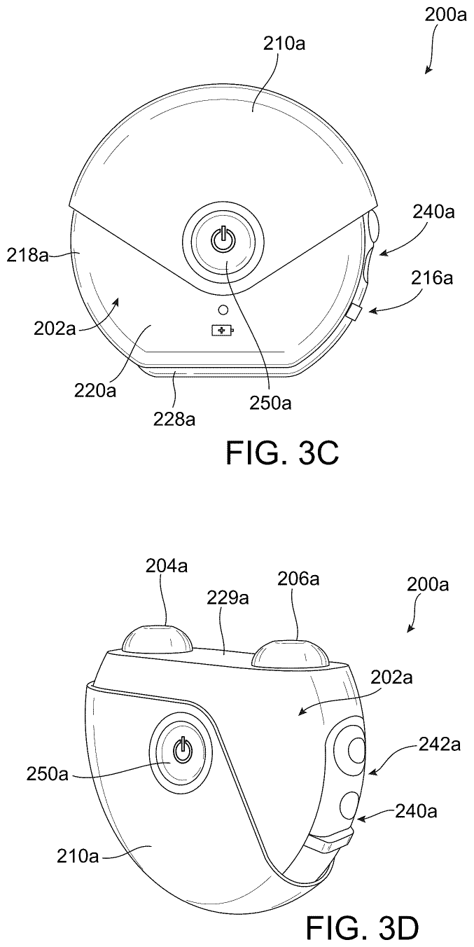

Referring now to FIGS. 3A and 3B, one embodiment of a stimulator 200 comprises a housing 202 and first and second electrodes 204, 206 extending from one surface of housing 202. Electrodes 204, 206 are applied to a surface of the user's body, during which time stimulator 200 may be held in place by straps, frames, collars or the like, or the stimulator 200 may be held against the user's body by hand.

Housing 202 contains the electronic components, signal generator and energy source (not shown) that are used to generate the signals that drive electrical impulses through electrodes 204, 206. However, in other embodiments, the electronic components that generate the signals may be in a separate housing or device, such as a mobile device. Furthermore, other embodiments may contain a single electrode or more than two electrodes.

Housing 202 comprises upper and lower portions 212, 214 and a cover 210 disposed between upper and lower portions 212, 214 for protecting electrodes 204, 206 from the external environment. Cover 210 also ensures that electrodes 204, 206 will not contact a user's tissue when the device is not intended to be used (e.g., in the event that the device is accidently turned ON and electric current is passed through electrodes when not in use). In certain embodiments, cover 210 is rotatably coupled to housing 202 such that it can be moved between a first position, wherein the electrodes 204, 206 are exposed for stimulation, and a second position, wherein the electrodes are housed and protected within the cover 210. Cover 210 may comprise any suitable material, such as polyphenylene ether (PPE), plastic, or other polymers.

Lower portion 214 of housing 202 preferably includes curved side surfaces 216, 218 with substantially planar surfaces 220 therebetween to form an overall disc-like shape that is cut off on the upper and lower portions of the disc. Lower portion 214 also includes a substantially planar bottom surface 228 that includes a control panel 240 (discussed below).

Similarly, upper portion 212 of housing preferably comprises curved side surfaces 224 and a substantially planar upper surface 229. Electrodes 204, 206 extend outward from upper surface 229. Upper portion 212 has a smaller width and length as lower portion 214 to form a groove 236 therebetween. Cover 210 rotates within groove 236. Upper and lower portions 212, 214 are preferably coupled to each other within housing 202. Alternatively, they may be molded together and formed as an integral component.

Control panel 240 may include a number of user controls and/or device status indicators. In alternative embodiments, the controls and status indicators are located on a separate device, such as a mobile device, that is wirelessly (e.g. Bluetooth or the like) coupled to stimulator 200.

In a preferred embodiment, control panel 240 includes intensity controls 242 for controlling the level of intensity or amplitude of the electrical impulses generated by stimulation 200. Intensity controls 242 extend outward from lower surface 228 so that the user be tactically identify and control intensity controls 242.

Control panel 240 may further comprise a battery life indicator 244 and/or a dose duration indicator 246. These indicators may include, for example, LEDs or other light sources, to facilitate identification by the user. The dose duration indicator 246 provides an indication of the time remaining on a single dose of electrical stimulation. In certain embodiments, stimulation 200 is configured to automatically cease the generation of the electrical impulse when the duration of the single dose has been completed.

Stimulator 200 may further include a power control 250 for turning ON the device. Power control 250 may also include an LED or other light source for illuminating power control 250 when the device has been turned ON. In one embodiment, power control 250 is located on side surface 200, although it will be understood that power control 250 may be located on control panel 240 or elsewhere on stimulator 200.

In certain embodiments, stimulator 200 further includes a charging pad 260 coupled to a suitable connector for providing power to stimulator 200 and/or recharging the battery within stimulator 200. Charging pad 260 may comprise any suitable charging source, such as an inductive charging source that provides power via inductive transmission through the lower surface 228 of housing 202.

Stimulator 200 may further include a gel pad 270 that includes a conductive gel 272, 274 positioned to contact electrodes 204, 206 when gel pad 270 is positioned over upper surface 229. Gel pad 270 is configured to apply a coating of electrically conductive gel to the surfaces of electrodes 204, 206 to facilitate conduction of the electrical impulses through an outer skin surface of the user.

The housing 202 may comprise plastic, metal, rubber, or other materials. The housing 202 may be rigid, elastic, resilient, or flexible. The housing 202 may be included in, or embodied as, a phone, a tablet, a laptop, a phone/tablet/laptop case, a patch, an adhesive bandage, a strip, an anklet, a belt, a bracelet, a necklace, a garment, a pad, a ring, a mattress, a pillow, a blanket, a robot, a surgical instrument, a stimulator, an infusion device, or others. The housing 202 may be embodied as described in US Patent Application Publication 20140330336 and U.S. Pat. Nos. 8,874,205, 9,174,066, 9,205,258, 9,375,571, and 9,427,581, all of which are incorporated entirely herein by reference for all purposes as if copied and pasted herein.

Electrodes 204, 206 may comprise a substantially solid conducting material (e.g., metal such as stainless steel, platinum, or a platinum-iridium alloy), which is possibly flexible in some embodiments. However, in other embodiments, the electrodes may have many other sizes and shapes, and they may be made of other materials. The electrodes preferably have a dome-shape with a rounded distal surface, although they may have the shape of a screw that is flattened on its tip. Pointing of the tip would make the electrode more of a point source, such that the equations for the electrical potential may have a solution corresponding more closely to a far-field approximation. Rounding of the electrode surface or making the surface with another shape will likewise affect the boundary conditions that determine the electric field.

In other embodiments, electrodes 204, 206 may be housed within housing 200. In these embodiments, housing includes an outer contact surface, such as a fluid permeable material that allows for passage of current through the permeable portions of the material. In these embodiments, a conductive medium (such as a gel) is preferably situated between the electrode(s) and the permeable interface. The conductive medium provides a conductive pathway for electrons to pass through the permeable interface to the outer surface of the interface and to the user's skin.

In other embodiments, the interface is made from a very thin material with a high dielectric constant, such as material used to make capacitors. For example, it may be Mylar having a submicron thickness (preferably in the range about 0.5 to about 1.5 microns) having a dielectric constant of about 3. Because one side of Mylar is slick, and the other side is microscopically rough, two different configurations are contemplated: one in which the slick side is oriented towards the user's skin, and the other in which the rough side is so oriented. Thus, at stimulation Fourier frequencies of several kilohertz or greater, the dielectric interface will capacitively couple the signal through itself, because it will have an impedance comparable to that of the skin. Thus, the dielectric interface will isolate the stimulator's electrode from the tissue, yet allow current to pass. In one embodiment, non-invasive electrical stimulation of a nerve is accomplished essentially substantially capacitively, which reduces the amount of ohmic stimulation, thereby reducing the sensation the user feels on the tissue surface. This would correspond to a situation, for example, in which at least 30%, preferably at least 50%, of the energy stimulating the nerve comes from capacitive coupling through the stimulator interface, rather than from ohmic coupling. In other words, a substantial portion (e.g., 50%) of the voltage drop is across the dielectric interface, while the remaining portion is through the tissue.

In certain embodiments, stimulator 200 includes an electronic filter, such as a low-pass filter that filters out or eliminates high frequency components from the signal to smooth out the signal before it reaches the electrodes 204, 206. The low-pass filter may comprise a digital or analog filter or simply a capacitor placed in series between the signal generator and the electrode/interface. When the signal is generated, energy switching and electrical noise typically add unwanted high frequency spikes back into the signal. In addition, the pulsing of the sinusoidal bursts may induce high frequency components in the signal. By filtering the signal just before it reaches the electrodes, a smoother, cleaner signal is applied to the user, thereby reducing the pain and discomfort felt by the user and allowing a higher amplitude to be applied to the user. This allows a sufficiently strong signal to be applied to reach a deeper nerve, such as the vagus nerve, without causing too much pain and discomfort to the user at the surface of their skin.

Referring now to FIGS. 3C and 3D, another embodiment of a stimulator 200a

comprises a housing 202a and first and second electrodes 204a, 206a extending from one surface of housing 202a. As in the previous embodiment, housing 202a contains the electronic components, signal generator and energy source (not shown) that are used to generate the signals that drive electrical impulses through electrodes 204a, 206a.

Stimulator 200a comprises a cover 210a for protecting electrodes 204a, 206a from the external environment. Cover 210 also ensures that electrodes 204a, 206a will not contact a user's tissue when the device is not intended to be used (e.g., in the event that the device is accidently turned ON and electric current is passed through electrodes when not in use). In certain embodiments, cover 210a is rotatably coupled to housing 202a such that it can be moved between a first position (FIG. 3D), wherein the electrodes 204a, 206a are exposed for stimulation, and a second position (FIG. 3C), wherein the electrodes are housed and protected within the cover 210. Cover 210a may comprise any suitable material, such as polyphenylene ether (PPE), plastic, or other polymers.

Housing 202a preferably includes curved side surfaces 216a, 218a with substantially planar surfaces 220a therebetween to form an overall disc-like shape that is cut off on the upper portion of the disc. A control panel 240a may be included on one of the side surfaces 216a. Control panel 240a includes a number of user controls and/or device status indicators. In alternative embodiments, the controls and status indicators are located on a separate device, such as a mobile device, that is wirelessly (e.g. Bluetooth or the like) coupled to stimulator 200a.

In a preferred embodiment, control panel 240a includes intensity controls 242a for controlling the level of intensity or amplitude of the electrical impulses generated by stimulator 200a. Intensity controls 242a extend outward from side surfaces 216a so that the user may tactically identify and control intensity controls 242a.

Control panel 240a may further comprise a battery life indicator and/or a dose duration indicator (not shown). These indicators may include, for example, LEDs or other light sources, to facilitate identification by the user.

Stimulator 200a may further include a power control 250a for turning ON the device. Power control 250a may also include an LED or other light source for illuminating power control 250a when the device has been turned ON. In one embodiment, power control 250a is located on planar surfaces 220a, although it will be understood that power control 250a may be located on control panel 240a or elsewhere on stimulator 200a.

In certain embodiments, stimulator 200a further includes a charging pad (not shown) coupled to a suitable connector for providing power to stimulator 200a and/or recharging the battery within stimulator 200a. The charging pad may comprise any suitable charging source, such as an inductive charging source that provides power via inductive transmission through the lower surface 228a of housing 202a. Stimulator 200 may further include a gel pad (not shown) that includes a conductive gel positioned to contact electrodes 204a, 206a when gel pad is positioned over upper surface 229a.

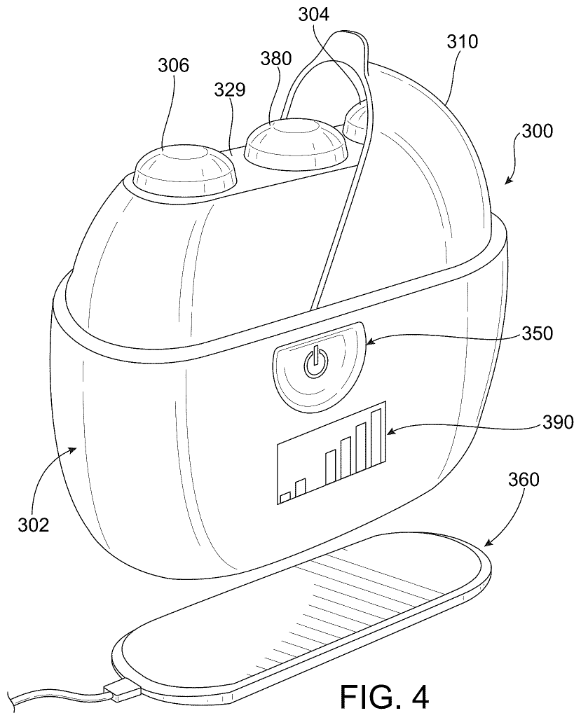

Referring now to FIG. 4, another embodiment of a stimulator 300 has a similar construction as stimulator 200 described above. In addition, stimulator 300 includes a sensor 380 extending from upper surface 329 of housing 302. Sensor 380 is preferably located between electrodes 304, 306, although it will be recognized that sensor 380 may be positioned in other locations on housing 302. For example, sensor 380 may be positioned on one of the side surfaces of housing 202, on the bottom surface 328 of housing, or electrodes 304, 306 may be positioned closer together such that sensor 380 is positioned on either side of electrodes 304, 306.

In certain embodiments, sensor 380 comprises a heart pulse sensor that detects the heart pulse of the user when the sensor 380 is placed in contact with, or near, the outer skin surface of the user. As discussed above, the heart pulse sensor detects that the sensor is close to, or adjacent, a source of heart pulse, such as the carotid artery in the user's neck or the radial artery in the wrist. The heart pulse sensor may be any suitable sensor known in the art, for detecting the heart pulse of a user, such as an infrared sensor, optical sensor, tactile sensor, a photoplethysmography (PPG) sensor or the like. The heart pulse sensor may, for example, detect the change in volume of a blood vessel that occurs when the heart pumps blood. Alternatively, the heart pulse sensor may detect vibrations, sounds or other indications that the sensor 380 is located adjacent to, or near, the user's heart pulse.

Sensor 380 is configured to generate an output that indicates the proximity of a heart pulse in the user. The output may be generated and transmitted via wire, wirelessly, or waveguide, to a control unit within stimulator 300, a mobile device, processor, server, or other logic or computing device. This output provides an indication that electrodes 304, 306 are positioned optimally to modulate the target nerve, e.g., the vagus nerve.

Stimulator 300 further includes a position indicator 390 coupled to sensor 380, the control until within stimulator 300, or a separate device, and configured to provide indication of the position of the stimulator relative to the heart pulse within the user. As discussed above, position indicator is configured to generate an alert when sensor 380 has detected the target nerve. The alert may be, for example, a visual, tactile or audial alert, that provides the user with an indication that the sensor 380 has detected the target location.

In certain embodiments, position indicator 380 is configured to transmit an alert that is associated with the magnitude of the heart pulse. In other embodiments, position indicator 390 is further be configured to provide a second alert when the magnitude of the heart pulse reaches a threshold level associated with optimal positioning of the sensor 380 and/or electrodes 304, 306.

Stimulators 200, 200a and 300 may include additional sensors, such as, for example, biosensors, feedback sensors, chemical sensors, optical sensors, acoustic sensors, vibration sensors, motion sensors, fluid sensors, radiation sensors, temperature sensors, motion sensors, proximity sensors, fluid sensors, or others. The sensors may generate an output, such as one or more outputs, which are communicated, via wire, wirelessly or waveguide, to the stimulator 200, a mobile device, processor, server, or other logic or computing device. The output may be used as an input to one or more of the foregoing devices to forecast or avert an imminent onset or predicted upcoming onset of a symptom, episode, condition or disease. For example, as disclosed in U.S. Patent App. Pub. No. 2017/0120052, which is incorporated herein by reference in its entirety for at least these purposes as if copied and pasted herein, as disclosed herein, and for all purposes as if copied and pasted herein, such as all structures, all functions, and all methods of manufacture and use, as disclosed therein.

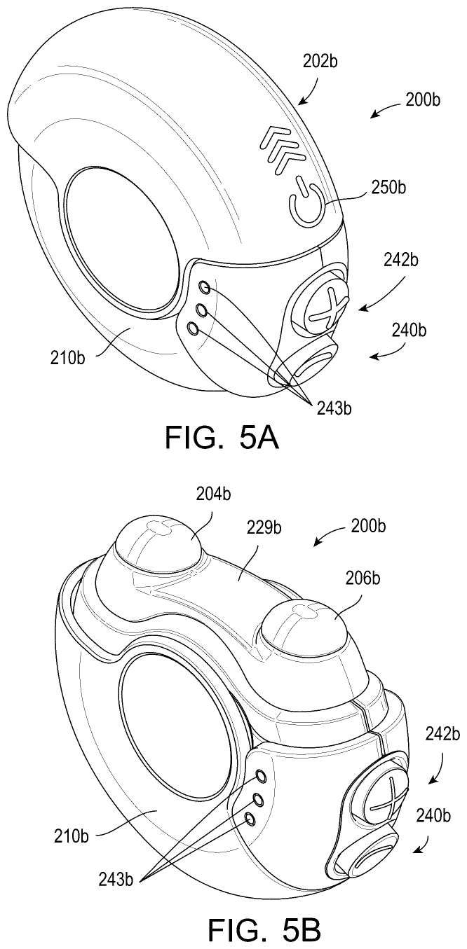

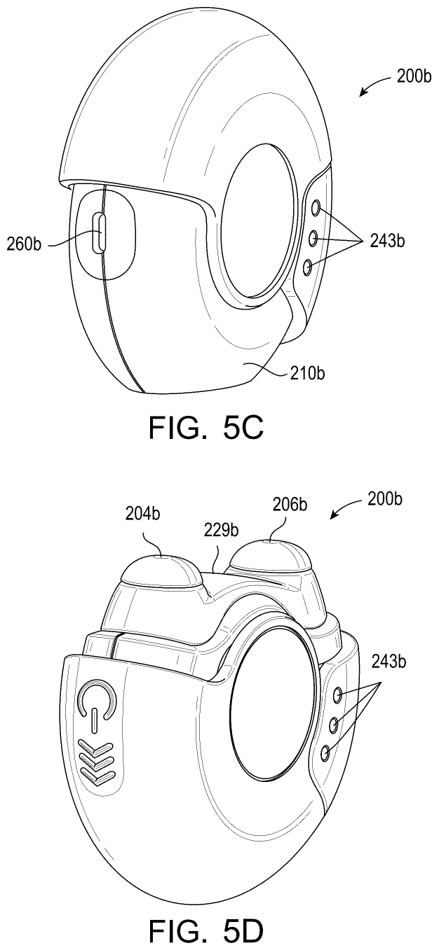

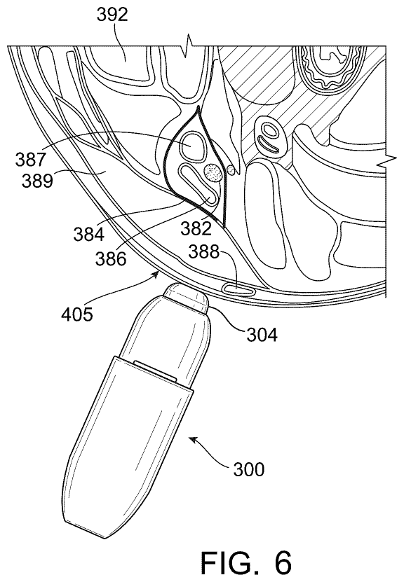

FIG. 6 provides a more detailed view of use of stimulator 300 when positioned to stimulate the vagus nerve at the neck location. The anatomy shown in FIG. 6 is a cross-section of half of the neck at vertebra level C6. The vagus nerve 382 is identified in FIG. 5, along with the carotid sheath 384 that is identified there in bold peripheral outline. The carotid sheath encloses not only the vagus nerve, but also the internal jugular vein 386 and the common carotid artery 387. Structures that may be identified near the surface of the neck include the external jugular vein 388 and the sternocleidomastoid muscle 389, which protrudes when the patient turns his or her head. Additional organs in the vicinity of the vagus nerve include the trachea 392, thyroid gland 393, esophagus 394, scalenus anterior muscle 395, scalenus medius muscle 396, levator scapulae muscle 397, splenius colli muscle 398, semispinalis capitis muscle 399, semispinalis colli muscle 401, longus colli muscle and longus capitis muscle 402. The sixth cervical vertebra 403 is shown with bony structure indicated by hatching marks.

In use, upper surface 399 of stimulator 300 is positioned near the outer skin surface 405 of the neck of the patient such that electrodes 304, 306 are in contact with surface 405. In certain embodiments, sensor 380 (not shown in FIG. 5) will also be in contact with skin surface 405. The user turns the device ON and moves the stimulator 300 along skin surface 405 until sensor 380 detects the heart pulse within carotid artery 387. Once the heart pulse has been detected, stimulator 300 is in the optimal position to transmit electrical impulses through electrodes 304, 306 to vagus nerve 382.

In certain embodiments, sensor 380 detects the magnitude of the heart pulse and generates a signal associated with such magnitude. In these embodiments, the user may elect to continue to reposition stimulator 300 until the magnitude of the heart pulse reaches a threshold level. Indicator 390 may be configured to provide a second alert to the user that such position has been reached.

Stimulation may be performed on the left or right vagus nerve or on both of them simultaneously and alternately. The position and angular orientation of the device are adjusted about that location until the user perceives stimulation when current is passed through the stimulator electrodes. The applied current is increased gradually, first to a level wherein the user feels sensation from the stimulation. The energy is then increased, but is set to a level that is less than one at which the user first indicates any discomfort. Straps, harnesses, or frames may be used to maintain the stimulator in position. The stimulator signal may have a frequency and other parameters that are selected to produce a therapeutic result in the user, i.e., stimulation parameters for each user are adjusted on an individualized basis. Ordinarily, the amplitude of the stimulation signal is set to the maximum that is comfortable for the user, and then the other stimulation parameters are adjusted.

The stimulation is then performed with a sinusoidal burst waveform like that shown in FIG. 2. As seen there, individual sinusoidal pulses have a period of t, and a burst consists of N such pulses. This is followed by a period with no signal (the inter-burst period). The pattern of a burst followed by silent inter-burst period repeats itself with a period of T. For example, the sinusoidal period τ may be between about 50-1000 microseconds and a frequency of about 1-20 kHz, preferably between about 100-400 microseconds and a frequency of about 2.5-10 kHz, more preferably about 133-400 microseconds and a frequency of about 2.5-7.5 kHz and even more preferably about 200 microseconds and a frequency of about 5 kHz; the number of pulses per burst may be N=1-20, preferably about 2-10 and more preferably about 5; and the whole pattern of burst followed by silent inter-burst period may have a period T comparable to about 5-100 Hz, preferably about 15-50 Hz, more preferably about 25-35 Hz and even more preferably about 25 Hz (a much smaller value of T is shown in FIG. 2C to make the bursts discernable). When these example values are used for T and τ, the waveform contains significant Fourier components at higher frequencies ( 1/200 microseconds=5000/sec), as compared with those contained in transcutaneous nerve stimulation waveforms.

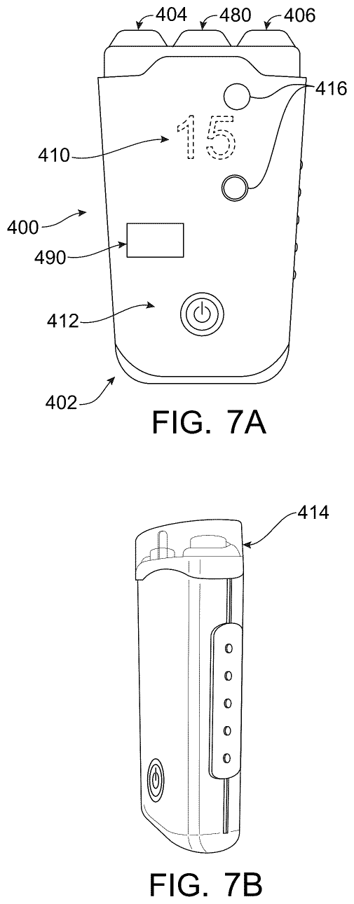

Referring now to FIGS. 7A and 7B, another embodiment of a stimulator 400 that includes a housing 402, a display 410, electrodes 404, 406, a power button 412 a cap 414 and a control button 416. In some embodiments, the neurostimulator 400 includes a speaker housed via the housing 400 and powered via the battery. In some embodiments, the neurostimulator 400 includes a microphone housed via the housing 402 and powered via the battery. The housing 402 houses a signal generator and a battery. The housing 402 is opaque, but can be transparent. The battery powers the signal generator and the display. The power button 408 turns the neurostimulator 400 on and off. The button 408 can be a mechanical button or a touch-enabled surface, which can be haptic or configured to receive a touch input, a slide input, a gesture input, or others. The electrodes 404, 406 contact a skin of a patient and conduct a stimulation energy, such as an electrical current, an electrical impulse, an actuation, or others, from the signal generator to the skin of the patient.

The display 410, which can present in monochrome, grayscale, or color, indicates a status of the neurostimulator 400, such as on, off, charging, dosage amount total, dosage amount remaining, stimulation time total, stimulation time remaining, or others. The display 410 can be of any type, such as a segment display, a liquid crystal display (LCD), an electrophoretic display, a field emission display (FED), or others, whether rigid, elastic, resilient, bendable, or flexible. The display 410 can be configured to receive a touch-input, including a gesture, a slide, or others.

The cap 414 is mounted to the housing 402, such as via snug fit, friction, fastening, mating, adhering, or others. The cap 414 is transparent, but can be opaque. The cap 414 covers and protects the electrodes 404, 406 from mechanical damage, interference, moisture, or others. The control button(s) 416 are operably coupled to the signal generator and is thereby configured to increase or decrease an intensity of the stimulation by controlling the signal generator. The control button(s) 416 can be a mechanical buttons or a touch-enabled surfaces, which can be haptic or configured to receive a touch input, a slide input, a gesture input, or others. The neurostimulator 400 can be charged via a charging station (not shown), whether in a wired, wireless, or waveguide manner.

Stimulator 400 further includes a sensor 480 preferably located between electrodes 404, 406, although it will be recognized that sensor 480 may be positioned in other locations on housing 402. For example, sensor 480 may be positioned on one of the side surfaces of housing 402, on the bottom surface of housing 402, or electrodes 404, 406 may be positioned closer together such that sensor 480 is positioned on either side of electrodes 404, 406.

In certain embodiments, sensor 480 comprises a heart pulse sensor that detects the heart pulse of the patient when the sensor 480 is placed in contact with, or near, the outer skin surface of the patient. As discussed above, the heart pulse sensor detects that the sensor is close to, or adjacent, a source of heart pulse, such as the carotid artery in the patient's neck or the radial artery in the wrist.

Stimulator 400 further includes a position indicator 490 coupled to sensor 480, the control until within stimulator 400, or a separate device, and configured to provide indication of the position of the stimulator relative to the heart pulse within the patient. As discussed above, position indicator is configured to generate an alert when sensor 480 has detected the target nerve. The alert may be, for example, a visual, tactile or audial alert, that provides the user with an indication that the sensor 380 has detected the target location.

Neurostimulator 400 can be a multi-use, hand-held, rechargeable, portable device comprising of a rechargeable battery, a set of signal-generating and amplifying electronics, and a control button for operator control of a signal amplitude. The device provides visible (display) and audible (beep) feedback on the device and stimulation status. A pair of stainless steel surfaces, which are a set of skin contact surfaces, allows a delivery of an electrical signal. The patient applies an electrode gel to the contact surfaces to maintain an uninterrupted conductive path from the contact surfaces to the skin on the neck of the patient. The stimulation surfaces are capped when not in use. The neurostimulator 400 can produce a low voltage electric signal including about five 5,000 Hz electric pulses (or less or more) that are repeated at a rate of 25 Hz (or less or more). A waveform of the electric pulses is approximately a sine wave with a peak voltage limited to about 24 volts (or less or more) when placed on the skin of the neck of the patient and a maximum output current of 60 mA (or less or more). The signal is transmitted through the skin of the neck to the vagus nerve. The neurostimulator 400 allows the patient to appropriately position and adjust a stimulation intensity as instructed a healthcare provider. Further details of appropriate waveforms and electrical signals and how to generate and transmit such signals to a desired nerve can be found in U.S. Pat. Nos. 8,874,205; 9,333,347; 9,174,066; 8,914,122 and 9,566,426, which are incorporated herein in their entireties by reference for at least these purposes as if copied and pasted herein, as disclosed herein, and for all purposes as if copied and pasted herein, such as all structures, all functions, and all methods of manufacture and use, as disclosed therein. Each dose can be applied for two minutes, after which the neurostimulator automatically stops delivering the neurostimulation. The neurostimulator 400 can allow for single or multiple uses or sessions. The neurostimulator can deliver a fixed number of treatments within a 24-hour period (or less or more). Once a maximum daily number of treatments has been reached, the neurostimulator 400 will not deliver any more treatments until a following 24-hour period expires. The neurostimulator can be charged via a charging station. The neurostimulator can allow for a fixed number of treatments within a defined time period, such as thirty one days or ninety three days, or some other period of time. A more complete description of systems for initially provisioning and refilling stimulator 400 can be found in U.S. patent application Ser. No. 16/229,299, filed Dec. 22, 2017, the complete disclosure of which is incorporated herein by reference for all purposes.

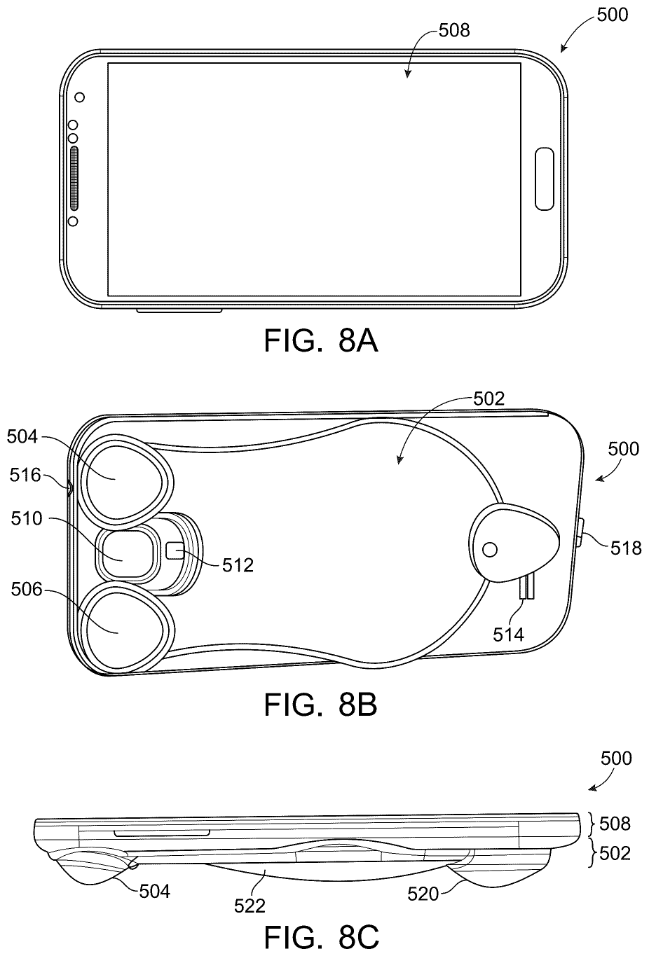

Another embodiment of an electrode-based stimulator 500 is shown in FIGS. 8A-8C. As shown, the stimulator comprises a smartphone with its back cover removed and and joined to a housing 502 that comprises a pair of electrode surfaces 504, 506 along with circuitry to control and energy the electrodes and interconnect with the smartphone. FIG. 8A shows the side of the smartphone 508 with a touch-screen. FIG. 8B shows the housing of the stimulator 502 joined to the back of the smartphone. Portions of the housing lie flush with the back of the smartphone, with windows to accommodate smartphone components that are found on the original back of the smartphone. Such components may also be used with the stimulator, e.g., the smartphone's rear camera 510, flash 512 and speaker 514. Other original components of the smartphone may also be used, such as the audio headset jack socket 516 and multi-purpose jack 518. Note that the original components of the smartphone shown in FIGS. 8A-8C correspond to a Samsung Galaxy smartphone, and their locations may be different for embodiments that use different smartphone models by different smartphone manufacturers. Note that tablets can be used as well.

FIG. 8C shows that several portions of the housing 502 protrude towards the back. The two electrode surfaces 504, 506 protrude so that they may be applied to the skin of the patient. The stimulator may be held in place by straps or frames or collars, or the stimulator may be held against the patient's body by hand. In some embodiments, the neurostimulator may comprise a single such electrode surface or more than two electrode surfaces.

A dome 520 also protrudes from the housing, so as to allow the device to lie more or less flat on a table when supported also by the electrode surfaces. The dome also accommodates a relatively tall component that may lie underneath it, such as a battery. Alternatively, the stimulation device may be emerged by the smartphone's battery. The belly 522 of the housing protrudes to a lesser extent than the electrodes and dome. The belly accommodates a printed circuit board that contains electronic components within the housing (not shown), as described below.

Stimulator 500 may also comprise a position sensor (not shown), such as one of the sensors describe above. The position sensor may, for example, be located in dome 520, or belly 522 of the housing. A more complete description of a stimulator for use with a mobile device can be found in commonly-assigned U.S. Pat. No. 9,375,571, the complete disclosure of which is incorporated herein by reference for all purposes.

### Embodiments Of The Stimuli Generating Devices

FIG. 1A, illustrates components involved in a conventional evoked potential (EP) measurement. The investigator initiates the generation of one or more sensory stimuli from a stimulator generator, such as a flash of light, an audio click, or bipolar transcutaneous electrical stimulation applied on the skin over the median, ulnar, peroneal, or posterior tibial nerve. The stimulus then activates visual, auditory, somatosensory, or pain exteroceptive sense organ receptors, respectively, in the subject of the measurement. The neural responses of the sensory receptors are then transmitted to structures within the central nervous system, which initially process the sensory information without conscious participation of the subject. However, those structures are also in communication with structures in the central nervous system that make it possible for the subject to subsequently become conscious of the sensory information, for example, by recognizing the novelty or significance of the stimulus.

As also shown in FIG. 1A, electrode sensors placed at well-defined locations on the scalp of the subject make it possible to measure electrical potentials that are evoked as the underlying structures of the central nervous system processes the sensory information, both unconsciously and consciously. Such neural processing generates ionic current flows within a brain of the subject that can be measured on the scalp. Actual measurement of the potentials is triggered by the activity of the sensory stimulus generator, so that the measured potentials are time-locked relative to the onset of the stimulus. When a transient response EP is measured, the EP waveform ordinarily consists of a series of peaks and valleys relative to the baseline potential, which are characterized by their amplitudes (positive or negative), as well as their times of occurrence relative to the stimulus (their latencies). The potentials that are so-measured are a mixture of the neural activity of structures involved in both the unconscious and conscious processing of the sensory information, as may be inferred by performing the EP measurement when the subject is or is not anesthetized, or awake versus asleep. Transient response EP data acquisition equipment may also be capable of averaging multiple successive evoked potentials (so as to increase the signal-to-noise of the EP data) and also automatically locate peaks or other features in the evoked potential waveform, such as a P300 peak that corresponds to a conscious evaluation on the part of the patient that the stimulus is interesting [KNIGHT R T, Scabini D. Anatomic bases of event-related potentials and their relationship to novelty detection in humans. J Clin Neurophysiol 15(1,1998):3-13; KECECI H, Degirmenci Y, Atakay S. Habituation and dishabituation of P300. Cogn Behav Neurol 19(3,2006):130-134].

Peaks and troughs in the transient response EP may often be identified by comparing their properties with those found in normative databases. Artifacts that appear in the EP may also be identified and preferably eliminated. In the case of electrical stimulation this may include a shock or stimulus artifact that is due to conduction through the skin from the stimulus to the recording electrode. It may also be a myogenic artifact that originates in scalp muscles in the vicinity of recording electrodes, or other muscles, and may be identified, for example, by the use of chemical muscle relaxants that cause the artifact to disappear.

The transient EP is produced as a response to a single brief stimulus, and for purposes of signal-averaging, the response is not evoked again until the potential has returned to its value prior to the stimulus. In contrast, a steady state EP is produced in response to stimuli that are repeated periodically, even though the potential may not have had time to return to its baseline value between stimuli. Such a steady-state EP will also exhibit a reproducible waveform, but because the waveform is dependent on factors such as the frequency of stimulus repetition, it is conventionally characterized in terms of its Fourier spectrum. However, it may also be characterized in terms of the amplitude and latency of peaks and troughs corresponding to the temporal summation of synaptic potentials [David REGAN. Distinction between the transient and steady-state responses of a system. Section 1.3, pp. 34-43 in: David REGAN. Human Brain Electrophysiology. Evoked potentials and evoked magnetic fields in science and medicine. New York: Elsevier Science Publishing Co., 1989; ZAKHAROVA I, Kornhuber M E. Facilitation of late somatosensory evoked potentials by electrical train stimuli. Neurosci Lett 557(Pt B, 2013):135-137].

Embodiments are shown in FIG. 1B, which are different from the prior art shown in FIG. 1A in several respects. First, in its non-invasive embodiment, it involves transcutaneous electrical stimulation applied to the skin over the vagus nerve in the neck, rather than the median, posterior tibial, or other nerves that are typically used in somatosensory evoked potential work. The vagus nerve at that cervical location comprises on the order of 100,000 axons that serve a large number of autonomic, sensory, and motor functions that are quantitatively and qualitatively greater than those served by the median, tibial, or other such nerves. Similarly, the vagus nerve axons at that cervical location serve many more functions than branches of the vagus nerve at other locations, such as auricular branch at the tragus of the ear. Therefore, the range of physiological effects that may be produced by the device shown in FIG. 1B is correspondingly greater than those that may be produced by the stimulation of other nerves or other vagal nerve branches.

As indicated in FIG. 1B the investigator initiates the operation of a vagus nerve stimulator, which generates cervical electrical stimulation through electrodes placed on the surface of the neck of the subject. The stimulation may involve the application of one or more superimposed stimulation waveforms, the parameters of which determine whether the stimulation preferably affects only receptors in the patient's skin, and/or whether the stimulation reaches the underlying vagus nerve. The somatosensory electrical stimulus used in FIG. 1A to stimulate nerves is ordinarily a monophasic square wave pulse having a duration of 100 microsecond to 1 second. In contrast, the devices shown in FIG. 1B use electrical stimulation waveforms that may be biphasic, bursting, sinusoidal and otherwise differ from monophasic square waves, in addition to the possibility that they involve the superposition of waveforms that are directed to the vagus nerve and cutaneous receptors. The stimulus waveforms produced by the devices shown in FIG. 1B may be either single-shot (to generate transient EP responses) or periodic (to generate steady-state EP responses).

FIG. 1B illustrates a closed-loop (feedback or biofeedback) system for acquiring evoked potential data. Unlike the system shown in FIG. 1A, the vagus nerve stimulator device may control and vary successive sensory stimuli, once the investigator has initiated its operation. Thus, in FIG. 1B, the vagus nerve stimulator may trigger the generation of the cervical sensory stimulus on its own, based upon its analysis of previous transient or steady-state evoked potentials that it had received from the scalp electrodes and/or from the analysis of other physiological data that it has received from other physiological sensors. Examples of such other physiological data are electrodermal voltages measured from sites such as the subject's hand or respiratory data that have been measured using impedance pneumography sensors.

The evoked potentials that are processed by such feedback methods may in some situations be generated primarily by the central nervous system structures for unconscious sensory processing. As an example of such feedback methods, the vagus nerve stimulator may vary a parameter of the stimulus waveform (e.g. amplitude, or frequency in the case of steady-state EP measurement), measure the resulting EP waveform, again vary the parameter based on that waveform measurement, and then repeat this procedure iteratively until it results in an EP waveform that exhibits preferred features that lie within some specified range. The use of such feedback would be particularly useful in establishing an initial set of stimulation parameter values for an individual, considering that different individuals may vary significantly with respect to the details of their preferred electrical stimulation waveforms. Furthermore, the plotting of some feature(s) of the EP waveform as a function of the varied parameters of the electrical stimulation waveform may be used to characterize the electrophysiology of the individual patient (stimulus/response gain, threshold, saturation, linearity or non-linearity, etc.). In fact, even the demonstrated ability to vary the EP waveform as a function of the parameters of the electrical stimulus waveform may be used to verify that the vagus nerve is in fact being stimulated, or that the position and/or orientation of the stimulation electrodes are optimal.

In other situations, the relevant features of the evoked potentials may be generated primarily by the central nervous system structures that are involved in conscious neural processing and control. As an example of that situation, the individual may consciously react to the sensations that result from the vagus nerve stimulation, as evidenced by the appearance of a P300 peak in his/her transient evoked potential. After detecting the P300 peak, the device can use that fact to vary the parameters of the next vagus nerve stimulation. For example, the P300 peak may appear once the stimulation amplitude reaches a sensory threshold that is recognized by the subject, or the properties of the P300 peak may change when the stimulation amplitude is so large that it produces pain. Because in that embodiment, the individual is consciously controlling the operation of the device via the P300 peak, this evoked potential application is a type of biofeedback, rather than purely automatic feedback.

Note that the type of biofeedback that is described above is different from other types of biofeedback, known as neurofeedback, that also measure potentials with scalp electrodes. This is because neurofeedback measures spontaneous (EEG) potentials, rather than evoked potentials. Thus, in neurofeedback, subjects are typically presented with an audio tone whenever their EEG contains significant EEG waves of a particular type (e.g., alpha, beta, high beta, theta, or sensorimotor). Some individuals can concentrate on the tone and then learn to voluntarily suppress and/or enhance the time spent in that EEG state, as evidenced by their ability to voluntarily increase or decrease the amplitude of the tone [John N. DEMOS. Getting Started with Neurofeedback. New York: W.W. Norton & Co., 2005. pp. 1-281].

Another novel feature of the system shown in FIG. 1B is that it may be used to train an individual to consciously and voluntarily control the “other physiological system” that is labeled in the figure. In such a biofeedback application, the skin at the subject's neck is stimulated in proportion to a previous or concurrently measured property of the “other physiological system” (e.g., electrodermal voltage measured on the subject's hand), such that the subject is made consciously aware of the magnitude of the measured physiological property through the magnitude of the skin stimulation. Alternatively, the stimulation applied to the subject's neck is a function of the features of the measured evoked potential (e.g., amplitude or latency of one or more particular EP waveform peaks or troughs). The subject then attempts to mentally control the magnitude of the skin stimulation, and thereby consciously control the magnitude of the measured physiological property through thought alone. The electrical signals that simulate cutaneous nerves within the skin may be analog signals that vary in some continuous way relative to the physiological property that is being transduced. Alternatively, the biofeedback signals may be digital, comprising recognizable coded pulse trains, as has been suggested in connection with tactile communication devices for the blind. For example, electrocutaneous signals with three discrete intensity levels and three discrete long-pulse durations can be discriminated.

It is understood that although the biofeedback component of FIG. 1B may be configured to use only electrical stimulation of the skin, the system may be configured to use additional sensory modalities as well, such as audio or visual biofeedback signals. However, the use of audio and visual sensory stimuli would ordinarily be used instead to evoke auditory or visual evoked potentials. Thus, FIG. 1B contains components “Other sensory stimuli” and “Other Sense Organs” that may refer to the stimulation of auditory or visual senses. In that situation, the vagus nerve stimulator/biofeedback device may also produce stimuli that stimulate vision or hearing (e.g., a flash of light or a click), thereby producing visual or auditory evoked potentials. Those “other sense organ” evoked potentials may then be measured via the scalp electrodes, and selected quantitative properties of the evoked potentials may then be automatically extracted by the vagus nerve stimulator/biofeedback device. Those properties may then be presented as a cutaneous sensation to the subject, via cervical electrical stimulation. In this embodiment, the subject becomes aware of the magnitude of the “other sense organ” evoked potential through the magnitude of the cutaneous sensation as biofeedback. It is understood that the cutaneous sensation itself may contribute to the evoked potential waveform, and preliminary experiments are used to distinguish which features of the EP waveform are due to the cutaneous stimulation and which are due to the “other sense organ”, such that the EP waveform feature used for the biofeedback arises primarily from stimulation of the “other sense organ.”

Generally, the devices shown in FIG. 1B will also be used to directly stimulate the vagus nerve, in addition to, or instead of, stimulating sensory nerves within the skin. As described below and in co-pending, commonly assigned patent application U.S. Ser. No. 13/222,087, entitled Devices and methods for non-invasive capacitive electrical stimulation and their use for vagus nerve stimulation on the neck of a patient, to SIMON et al. (which is hereby incorporated by reference), Applicant has developed a stimulator device that can noninvasively stimulate a vagus nerve directly in the patient's neck, without producing cutaneous discomfort to a patient. When the vagus nerve is being stimulated by the device, the quality of sensation in the patient's skin above the vagus nerve depends strongly on the stimulation current and frequency, such that when the currents are not much greater than the perception threshold, the cutaneous sensations may be described as tingle, itch, vibration, buzz, touch, pressure, or pinch. For situations in which the skin is being stimulated with a constant current and with a particular type of stimulation waveform that is described below, any such cutaneous sensation may be ignored by the patient, and the stimulator does not serve as an exteroceptive biofeedback device. In that case, the device resembles instead a physiological control device that may be used to stimulate structures of the central nervous system and/or “Other physiological systems”, via stimulation of the vagus nerve, as indicated in FIG. 1B. The particular structures of the central nervous system or other physiological systems that are affected by the vagus nerve stimulation depend on the parameters of the vagus nerve stimulation, which are selected to stimulate the particular system. Direct electrical stimulation of the vagus nerve will itself generate evoked potentials, as the resulting vagal action potentials and their sequelae propagate within the central nervous system.

In certain aspects, the measurement of an evoked potential as described above may be used to optimize non-invasive stimulation of the vagus nerve with, for example, one of the devices described below. Given that a particular evoked potential can be quantified that represents stimulation of the vagus nerve, the operator can use this measurement to confirm that the action potentials have been created in the vagus nerve during electrical stimulation. In this manner, the operator may, for example, vary a characteristic of the electrical impulses generator by the vagus nerve stimulator in order to ensure that such stimulation is effectively stimulating the vagus nerve at a therapeutic level. For example, if such stimulation does not initially generate the evoked potentials that would confirm the firing of the action potentials in the vagus nerve, the operator may vary aspects of the signal, such as the amplitude, frequency, pulse width and/or duty cycle until such an evoked potential is generated. In addition or alternatively, the operator may vary the placement or orientation of the device on the subject's neck to ensure proper stimulation of the vagus nerve. As another alternative, the operator may position the vagal nerve stimulator on the other side of the patient's neck (left to right or vice versa) in an attempt to optimize the stimulation.

In a more general embodiment of the system shown in FIG. 1B, a cutaneous biofeedback signal may be superimposed upon the electrical stimulation waveform that preferentially stimulates the vagus nerve directly. Thus, in addition to the mechanisms described in the previous two paragraphs, the stimulation waveform may also contain a time-varying signal with frequency components that are designed specifically to stimulate cutaneous nerves. The biofeedback signal will vary as a function of the physiological parameter that is being sensed by the physiological sensor (e.g., evoked potential feature or skin conductance level). The biofeedback signal may be a continuous analog signal, or it may be a digital signal, e.g., with three discrete intensity levels and three discrete long-pulse durations that can be discriminated. The patient may then consciously respond to the biofeedback signal, for example, by relaxing or tensing skeletal muscles or by eliciting a relaxing or agitated emotional response, thereby modulating the tone of the sympathetic nervous system [COSTA F, Biaggioni I. Role of adenosine in the sympathetic activation produced by isometric exercise in humans. J Clin Invest. 93(1994):1654-1660; KREIBIG S D. Autonomic nervous system activity in emotion: a review. Biol Psychol 84 (3,2010):394-421].

The three mechanisms illustrated in FIG. 1B (biofeedback, artificial interoceptive sensation, and direct stimulation via the vagus nerve) will collectively modulate the central nervous system or other physiological systems, interacting with one another to determine the value of the sensed physiological signal or feature of the evoked potential. Part of the interaction is determined by the manner in which the vagus nerve stimulator/biofeedback device/feedback controller is programmed. For example, direct stimulation of the physiological system via the vagus nerve may be programmed to follow and amplify or enhance changes that occur as a result of biofeedback. An embodiment of that example would occur when the individual uses galvanic skin response biofeedback alone to consciously reduce sympathetic tone through muscular and emotional modulation, whereupon the device in FIG. 1B senses that reduction through its programming and then amplifies the effect by increasing parasympathetic tone after a brief time delay, by directly stimulating vagal parasympathetic efferent nerve fibers.

In this example, it is clear what the biofeedback effect is initially (reduction of sympathetic tone), and the vagus stimulation is only applied thereafter to amplify it (stimulation of vagal parasympathetic fibers). In other embodiments that are disclosed herein, both biofeedback and vagus nerve stimulation are performed simultaneously, and mathematical modeling is used to infer the effects that are due to the biofeedback, thereby allowing the device to also infer the intentions of the individual and apply the vagus nerve stimulation accordingly. Consequently, the whole device shown in FIG. 1B has more functionality than its individual parts simply added together.

In certain embodiments, the system comprises software and hardware components to fix the parameters of the electrical impulses after they have been optimized. In one aspect, feedback provided by the physiological sensor optimizes the signal applied to the nerve. Once the signal has been optimized, the software and hardware components of the system fix the electrical impulse based on the parameters that have been sensed by the physiological sensor. The signal generator will then apply the fixed electrical impulse to the patient. For example, the physician may be able to optimize the electrical impulse in the hospital or office setting by applying electrical impulses and measuring their effect on certain body parameters. The impulses can then be varied either manually or automatically until the effect is optimized. If the stimulator is implanted, the signal generator may automatically apply the optimized electrical impulse to the patient at certain times throughout the day, or it may be designed to only apply the electrical impulses when activated by the patient. If the stimulator is a non-invasive device, the patient self-treats and applies the optimized electrical impulses according to the treatment algorithm set up by the physician.

The patient may be tested (without feedback or biofeedback) by stimulating “other sense organs” or the cervical cutaneous senses in FIG. 1B, and measuring the corresponding evoked potentials, over an extended period of time (e.g., visual, auditory, or traditional somatosensory EPs, as reviewed in COPPOLA G, Pierelli F, Schoenen J. Habituation and migraine. Neurobiol Learn Mem 92(2,2009):249-259). The patients who do not exhibit significant habituation in their evoked potentials, in response to the sensory stimulation over a prolonged period of time, are then subjected to an acute direct stimulation of the vagus nerve. The patient is then retested (again without feedback or biofeedback) by stimulating “other sense organs” and re-measuring the previously-measured evoked potentials (visual, auditory, or traditional somatosensory EPs). For some of the individuals (the “responders”), the effect of the intervening acute vagus nerve stimulation is to significantly reduce the magnitude of features of evoked potentials, thereby artificially effecting a form of EP habituation. Those individuals are therefore candidates for chronic treatment of their migraine headaches, by performing the vagus nerve stimulation on a regular basis, with the objective of reducing the duration, frequency and severity of symptoms associated with the disorder (e.g., migraine attacks, pain associated with fibromyalgia, etc.).

Vagus nerve stimulation may also be useful for the treatment of patients irrespective of whether the patient exhibits a deficit in the habituation of evoked potentials, and irrespective of whether the vagus nerve stimulation promotes the normalization of habituation of evoked potentials. In migraineurs, for example, the likely usefulness of the vagus nerve stimulation may more generally be based primarily upon the baseline characteristics of an evoked potential, measured during one or more phases of the migraine headache, particularly during the interictal phase. In fact, it is preferable to perform the measurements during multiple times throughout the interictal phase, in view of the changes in the evoked potential that occur throughout that phase. A method for using previously measured values of characteristics of the baseline evoked potential, to infer the likelihood of therapeutic success, is as follows. If the migraine attack is in progress, noninvasive vagus nerve stimulation is administered, and its effect on the reduction of headache pain is measured. The pain measurement may be based on self-reporting of the patient, or it may be based on an objective physiological measurement of pain. Note that evoked potentials themselves may be correlated with the level of pain and that EEG and autonomic physiological variables collectively (heart rate variability, electrodermal response) may also be measured as being correlated with the level of pain.

The measurement of pain may also be made following stimulation with multiple sets of vagus nerve stimulation parameters, in order to evaluate the stimulation parameters that have the greatest effect on the reduction of pain. After vagus nerve stimulation, the evoked potential may be measured again, and the features of the baseline evoked potential may then be compared with features of the post-stimulation evoked potential. Changes in the evoked potential may involve differences in amplitudes and latencies of peaks and troughs, which are of potential predictive value. When such measurements are performed on populations of migraineurs and control normal individuals, statistical methods may then be used to determine which features of the pre- and post-stimulation evoked potentials, as well as their differences, are most closely related to the reduction of pain in the migraineur. The statistical methods may also be used to predict which parameters of the vagus nerve stimulation have the greatest effect on the reduction of pain and on the features of the pre- and post-stimulation evoked potentials. The vagus nerve stimulation may then be re-applied to the patient, with a different set of stimulation parameters, selected on the basis of the relation between those parameters and pain reduction, as well as on characteristics of the pre- and/or post-stimulation evoked potentials.

The vagus nerve stimulation may also be used as a prophylaxis to reduce the frequency or severity of migraine attacks. In that case, the vagus nerve stimulation is applied to the patient over a prolonged period of time, and its quantitative effects on the frequency and severity of the migraine attacks is measured. When such measurements are performed on populations of migraineurs and control normal individuals, statistical methods may then be used to determine which features of the initial pre- and post-stimulation evoked potentials, as well as their differences, are most closely related to reduction in the chronic frequency and severity of migraine attacks. Thereafter, the likelihood that vagus nerve stimulation will be successful in treating a migraineur chronically may be inferred from the measured features of his/her initial pre- and post-stimulation evoked potentials, as well as differences between the pre- and post-stimulation evoked potentials.

In certain embodiments, the sensory stimulator comprises an auditory stimulator and a visual stimulator. In an exemplary embodiment, the auditory stimulator is configured to deliver binaural tones or beats to the brain of the user. Binaural beats (BNB) are defined as an auditory illusion created when two tones with different frequencies are delivered separately into each ear of the user. The brain perceives a third tone or a “binaural beat” which is the difference between the two frequencies.

In embodiments, the binaural tones have a carrier frequency of about 10 Hz to about 100 Hz, or about 20 Hz to about 80 Hz, or about 30 Hz to about 50 Hz, or about 40 Hz. The binaural tones have a beat frequency of about 0.1 Hz to about 10 Hz, or about 0.2 Hz to about 1 Hz, or about 0.4 Hz to about 0.6 Hz, or about 0.5 Hz. Lower frequencies (e.g., 0.5 −4 Hz delta waves) promote detailed memory consolidation and glymphatic clearance, characteristic of deep sleep (NREM N3), while higher frequencies (e.g., 25 to 50 Hz gamma waves) facilitate procedural memory consolidation and emotional processing, typically associated with REM sleep.

In embodiments, the visual stimulator is configured to deliver one or more light patterns to the user. In an exemplary embodiment, the light patterns are synchronized with the binaural tones. By delivering synchronized binaural tones and red light at these distinct frequencies (e.g., 0.5 Hz delta beat with soothing carrier, including 40 Hz gamma carrier if, or 40 Hz delta carrier), the system induces concurrent brainwave activities, enabling restorative and cognitive benefits within a single sleep session.

In certain embodiments, the light patterns may comprise waves that modulate at about 0.1 Hz to about 10 Hz, or about 0.2 Hz to about 1 Hz, or about 0.4 Hz to about 0.6 Hz, or about 0.5 Hz. The waves may flicker at about 10 Hz to about 100 Hz, or about 20 Hz to about 80 Hz, or about 30 Hz to about 50 Hz, or about 40 Hz.

In embodiments, the light waves may have a wavelength of about 380 nm to about 750 nm (i.e., visible light). In an exemplary embodiment, the wavelength may fall within a range of one or more specific colors, such as blue, red, green or the like. In one such embodiment, the wavelength falls in, or near, the red wavelength, or about 600 nm to about 750 nm, or about 610 nm to about 680 nm or about 620 nm to about 630 nm. The red light minimizes disruption of melatonin production in the body and provides circadian rhythm support.

In embodiments, the system further comprises one or more sensors coupled to the stimuli stimulator. The sensors are configured to detect one or more physiological parameters of the user. In an exemplary embodiment, the sensors comprise EEG sensors configured to measure voltage differences between pairs of electrodes positioned on a scalp of the user. These voltages may, for example, reflect summed electrical activity of neurons from the brain of the user. This summed electrical activity may represent differential electrical activity or the difference in voltage between two locations, which may, for example, represent a differential brainwave activity.

In embodiments, the system further comprises a computer readable media comprising non-transitory computer executable instructions which, when executed by at least one electronic processor, computes an effective brainwave frequency based on the differential brainwave activity detected by the sensors. In an exemplary embodiment, the computer readable media comprises non-transitory computer executable instructions which, when executed by at least one electronic processor synchronizes the binaural tones with the effective brainwave frequency.

In an exemplary embodiment, the stimuli generating device comprises a closed-loop, biofeedback system for enhancing sleep quality through synchronized auditory and visual stimulation. The system measures a user's brainwaves in real time using EEG sensors, processes the data to determine the effective brainwave frequency and sleep stage (e.g., NREM N1-N3, REM, or awake), and dynamically adjusts binaural tones and red light patterns to entrain the brain toward slower frequencies associated with deep sleep (e.g., 0.5-1 Hz). The synergistic effect of auditory (binaural tones) and visual (red LEDs) stimuli reinforces brainwave entrainment, reducing sleep onset time and increasing time in deep sleep stages. A three-axis accelerometer enhances REM sleep detection accuracy by confirming muscle atonia.

The system simultaneously entrains multiple brainwave frequencies, leveraging the brain's capacity to exhibit diverse brainwave activities concurrently. Lower frequencies (e.g., 0.5-4 Hz delta waves) promote detailed memory consolidation and glymphatic clearance, characteristic of deep sleep (NREM N3), while higher frequencies (e.g., 25-50 Hz gamma waves) facilitate procedural memory consolidation and emotional processing, typically associated with REM sleep. By delivering synchronized binaural tones and red light at these distinct frequencies (e.g., 0.5 Hz delta beat with soothing carrier, including 40 Hz gamma carrier if, or 40 Hz delta carrier), the system induces concurrent brainwave activities, enabling restorative and cognitive benefits within a single sleep session. This multi-frequency approach allows users to achieve equivalent sleep benefits in fewer hours of unconsciousness compared to natural sleep, addressing an unmet need for efficient sleep optimization. The system also can incorporate 40 Hz carrier tones and 40 Hz pulsing light during deep sleep to promote neurological health by clearing waste materials, such as beta-amyloid plaques.

In a preferred embodiment, the system comprises a wearable device, such as a headband or the like, with one or more of the following integrated components: (1) one or more speakers configured to deliver binaural tones for auditory stimulation. (2) one or more eye coverings, such as a mask, that includes one or more light emitters, such as light emitting diodes (LEDs) or the like. In embodiments, the LEDs emit red light (620-630 nm) for visual stimulation, avoiding melatonin disruption. (3) EEG Sensors, such as one or more dry electrodes (forehead and occipital, comb-style for hair) configured to measure differential brainwave activity. One electrode can yield acceptable performance and the addition of more than one can allow for more differentiation of activity around the brain. (4) a processor, microcontroller, integrated chip or the like and associated electronics; (5) a data storage unit, software application or computer readable media comprising non-transitory computer executable instructions which, when executed by at least the processor, performs real-time signal processing (e.g., Fast Fourier Transform, filtering) to compute effective brainwave frequency and control stimuli. (5) an energy source, such as a rechargeable battery or the like; (6) a user interface to, for example, adjust volume, light intensity, and auditory balance via the headband or software application. (7) a data storage unit, software application or computer readable media comprising non-transitory computer executable instructions which, when executed by at least the processor logs data, and records brainwave and stimuli data for machine learning personalization; and (8) a motion detector, such as an accelerometer configured to confirms REM sleep via muscle atonia detection.

Optional embodiments include the eye mask being detachable and re-attachable, single-modality stimulation modes, e.g., audio only or visual only, and a non-headband configuration employing adhesive EEG electrodes, wireless earbuds, and an LED sleep mask wirelessly connected. The invention overcomes prior art limitations by integrating closed-loop biofeedback, synchronized audio-visual stimulation, simultaneous multifrequency entrainment, and personalized machine learning, offering a scalable, noninvasive solution for sleep enhancement and neurological health.

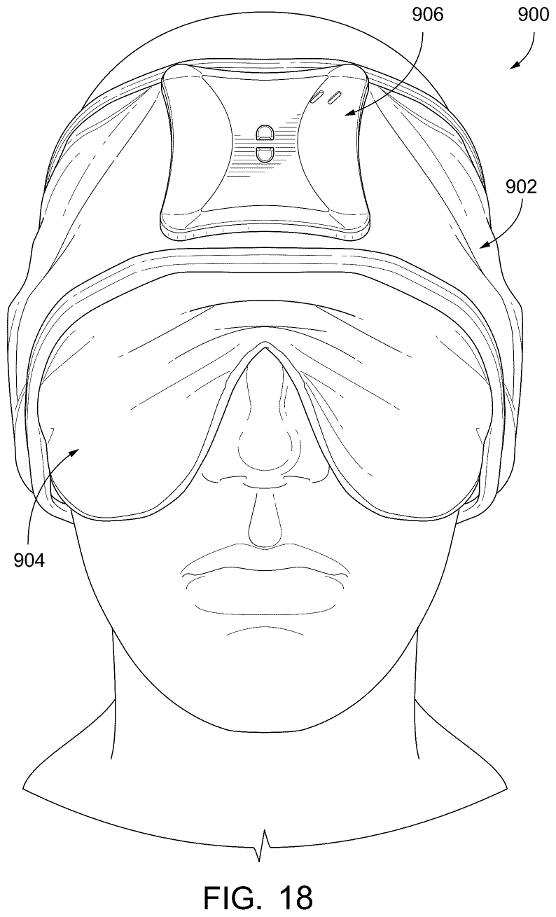

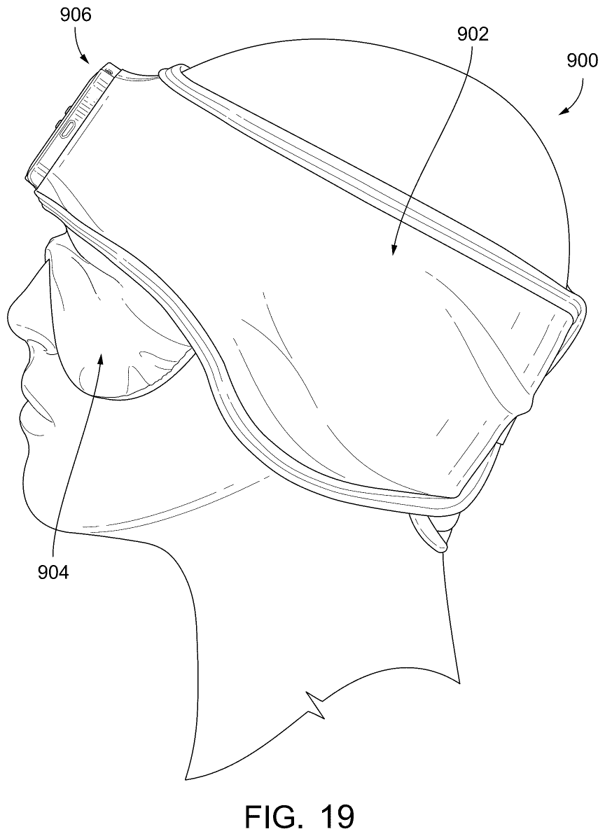

Referring now to FIGS. 18 and 19, a stimuli generation device or system 900 comprises a wearable device, such as a headband 902, an eye covering 904 and a controller 906 coupled to, or integrated within, the headband 902. In some embodiments, controller 906 may be remote from headband 902 and wirelessly coupled thereto. The system may comprise one or more of integrated speakers for delivering the auditory stimuli, light transmitters, such as LEDS or the like, for delivering the visual stimuli, and sensors, such as EEG sensors, for detecting a physiological parameter of the user. Alternatively, one or more of these components may be located remote to the system and wirelessly coupled thereto. The stimulator 906 may also include an accelerometer and the associated processor or electronics for synchronizing brainwave activity with the stimuli.

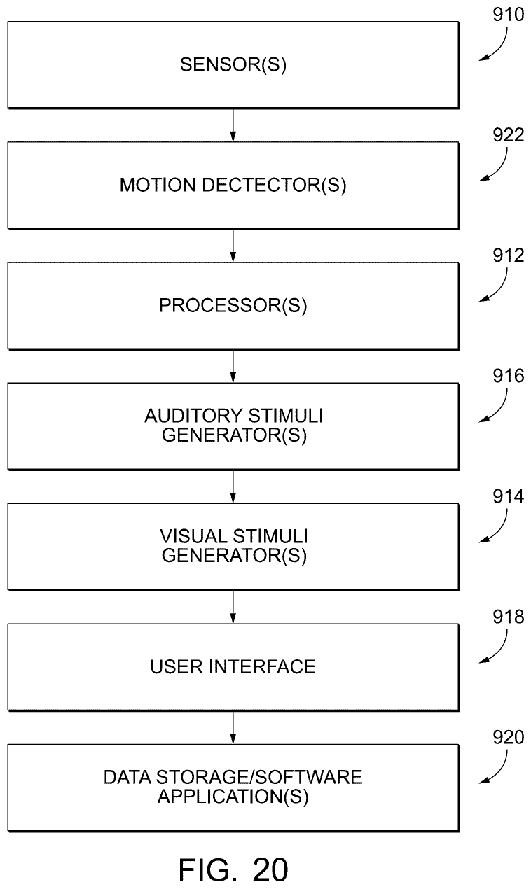

Referring now to FIG. 20, system 900 further includes one or more EEG sensors 910, such as two dry electrodes (e.g., forehead, occipital comb-style, PEDOT: PSS with rosinowax adhesives) that are configured to measure brainwave activity at about 200 Hz to about 300 Hz, or about 220 Hz to about 275 Hz or about 256 Hz. Sensors 910 may be housed in the controller 906 or the headband 902.

System 900 further comprises a processor and electronics 912, such as an ARM Cortex-M4 microcontroller (e.g., 256 KB RAM, 1 MB flash) that processes EEG signals via Fast Fourier Transform (FFT), smoothing, and bandpass filtering (e.g., about 0.5 Hz, to about 30 Hz) to compute the effective brainwave frequency and sleep stage.

System 900 further includes a rechargeable battery (not shown), such as a 400 mAh lithium-ion, 8-10 hours operation, USB-C rechargeable. The battery may be housed in the controller 906, the headband 902 or the eyemask 904.

System 900 further includes one or more visual stimuli generators 914 that are housed in eye mask 904. In an exemplary embodiment, the visual stimuli generators 914 comprise light transmitters, such as LEDs. In one such embodiment, the light transmitters comprise one or more red LEDs configured to deliver light at a wavelength of about 620-630 nm (max 10 cd/mQ, IEC 62471-compliant). The LEDs may emit gently varying light patterns synchronized with binaural tones, and/or 40 Hz flickers depending upon the EEG measures.

System further comprises one or more auditory stimuli generators 916, which are preferably housed within headband 902 (adjacent to, or near, the user's ears). In certain embodiments, auditory stimuli generators 916 includes speakers (not shown) configured to deliver binaural tones to one or both of the user's ears (e.g., a user perceives a tone which is the arithmetic difference of the left and right channels despite that tone not being played for either ear) for brainwave entrainment.

System 900 further comprises a user interface 918, which may be located on headband 902, controller 906 or eyemask 904. Alternatively, user interface 918 may be remotely located relative to system 900 and wirelessly coupled to controller 906. User interface 918 includes one or more controls for adjusting various parameters of the auditory and visual stimuli. In certain embodiments, user interface 918 comprises controls for adjusting the volume of the auditory stimuli and the intensity and/or balance of the visual stimuli. The user interface 918 may also include other controls, such as ON/OFF, or it may be configured to independently turn the auditory and visual stimuli ON/OFF (i.e., if the user chooses to use only one of them).

System 900 further includes a motion detector 922, such as a three-axis accelerometer configured to detect muscle detection or other movement of the user. This data is transmitted to the processor 912 or the software application 920.

System 900 further includes a data storage unit, software application or computer readable media 920 comprising non-transitory computer executable instructions which, when executed by at least one processor, logs data from the sensors and records brainwave and stimuli data for machine learning personalization. Data storage unit, software application or computer readable media 920 may also comprise non-transitory computer executable instructions which, when executed by at least one processor, determines whether the user is in REM sleep based on the motion data from the accelerometer and enhances sleep stage classification accuracy. These sleep stages may include: (1) NREM N1: 4-8 Hz theta and 8-13 Hz alpha; (2) NREM N2: 4-8 Hz theta, 11-16 Hz spindles, K-complexes; (3) NREM N3: 0.5-4 Hz delta (>20% epoch); (3) REM: 4-8 Hz theta, 13-30 Hz beta, 25-50 Hz gamma, 2-6 Hz sawtooth waves, spikes, muscle atonia; and/or (4) Awake: 8-13 Hz alpha, 13-30 Hz beta. The data storage unit or software application 920 may be housed within controller 906, or it may be remotely located relative to system 900 and wirelessly coupled to controller 906.

In certain embodiments, controller 906, processor 912 and/or software application 920 are configured to adjust the visual or auditory stimuli based on the effective brainwave frequency. For example, the binaural tones and/or red light may be modulated at a frequency slightly lower than the effective brainwave frequency to entrain slower frequencies.

In certain embodiments, controller 906, processor 912 and/or software application 920 are configured to adjust the visual or auditory stimuli for deep sleep optimization. For example, at 0.5-1 Hz (N3), the binaural tones may be slowed down to 0.5 Hz (including possibly with 40.25 Hz/39.75 Hz carriers (40 Hz gamma) for waste clearance) and the red light may be modulated to 0.5 Hz. The red light may also be modulated to flicker within a modulation envelope at 40 Hz, using pulse width modulation.

In certain embodiments, controller 906, processor 912 and/or software application 920 are configured to adjust the visual or auditory stimuli during REM sleep. For example, based on EEG spikes, which are detected by the sensors based on muscle atonia, the LEDs may flicker at 40 Hz and may be synchronized with the binaural tone envelope for neurological benefits.

In certain embodiments, controller 906, processor 912 and/or software application 920 are configured to dynamically adjust the visual or auditory stimuli as brainwaves slow. For example, the frequency of the auditory stimuli may be adjusted to within 0.5-1 Hz during this phase of sleep. A critical innovation is simultaneous multi-frequency entrainment, leveraging the brain's ability to exhibit concurrent brainwave activities. Applicant has discovered that lower frequencies (0.5-4 Hz delta) support detailed memory consolidation and glymphatic clearance during deep sleep, while higher frequencies (25-50 Hz gamma) facilitate procedural memory consolidation and emotional processing, typically seen in REM sleep. The system delivers binaural tones (e.g., 0.5 Hz beat for delta) with gamma carriers (40 Hz) and synchronized red light (e.g., 0.5 Hz or 40 Hz flicker), inducing these brainwave activities simultaneously. For example, during N3, delta entrainment enhances restorative functions, while gamma carriers promote memory replay, maximizing cognitive benefits. This multi-frequency approach allows users to achieve restorative and cognitive sleep benefits in fewer hours than natural sleep, as the system efficiently combines brainwave activities within a single session. The accelerometer ensures precise REM targeting, enhancing gamma stimulation efficacy.

In certain embodiments, controller 906, processor 912 and/or software application 920 are configured to provide carrier tone logic to support neurological health. For example, a 0.5 Hz binaural one may be delivered to the user at the deepest sleep stage with channels transmitting 40.25 Hz/39.75 Hz (a 40 Hz carrier). For sleep stages at higher predominate frequencies, the left and right channel tones may be increased asymmetrically so that the carrier tone is greater than 40 Hz when the binaural tone is greater than 0.5 Hz.

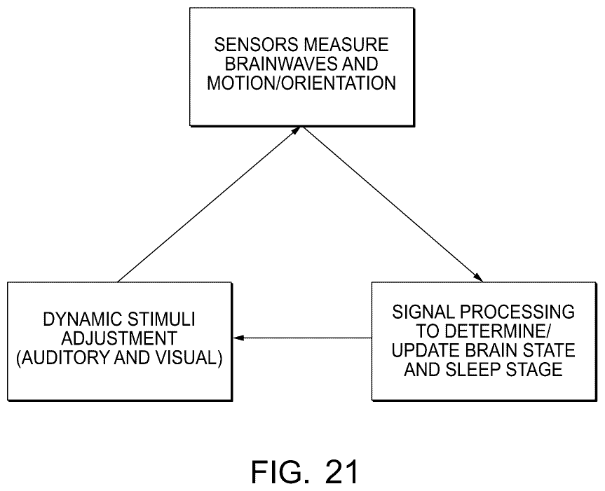

Referring now to FIG. 21, in operation, the user places headband 904 over his/her head such that the speakers 916 are positioned adjacent to, or near, the left and right ears. The user also places eyemask 904 over the eyes such that LEDs 914 are positioned over the left and right eyes. The EEG sensors are positioned around the patient's head within headband 904.

Once system 900 is in place, controller 906, processor 912 and/or software application 920 initiate the EEG sensors 910 to detect brainwave activity and accelerometer 922 to detect muscle atonia. Controller 906, processor 912 and/or software application 920 monitor brainwaves and motion/orientation and adjust stimuli in real time. Controller 906, processor 912 and/or software application 920 log the data for AI machine learning optimization of the therapy.

Applicant has discovered that the combination of nerve stimulation and visual/auditory stimulation provides a number of benefits to the user, including but not limited to, reduced sleep onset (10-20 minutes), increased N3 duration (20-30% more), neurological health via 40 Hz stimulation (i.e., beta-amyloid/tau reduction) and simultaneous delta (detailed memory) and gamma (procedural memory) benefits despite reduced sleep duration,

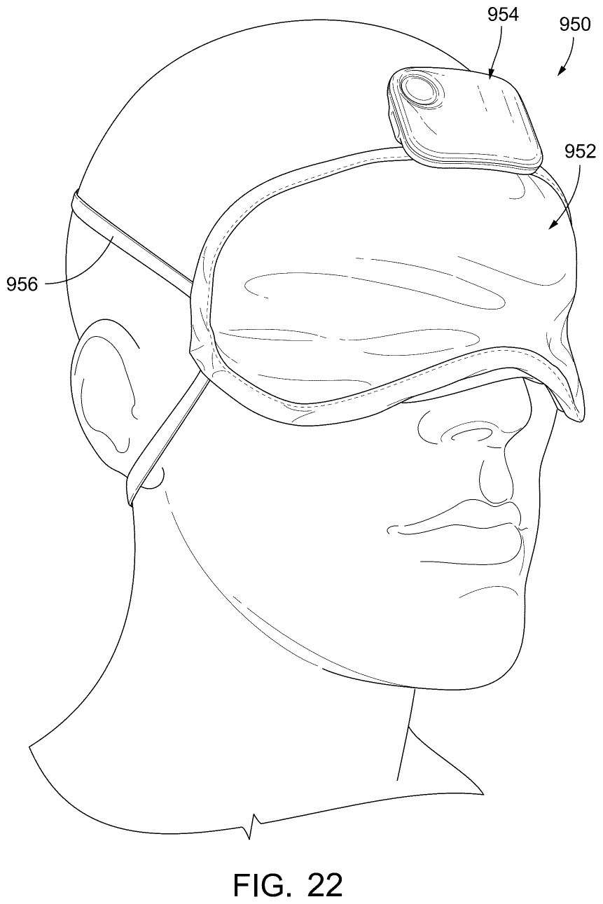

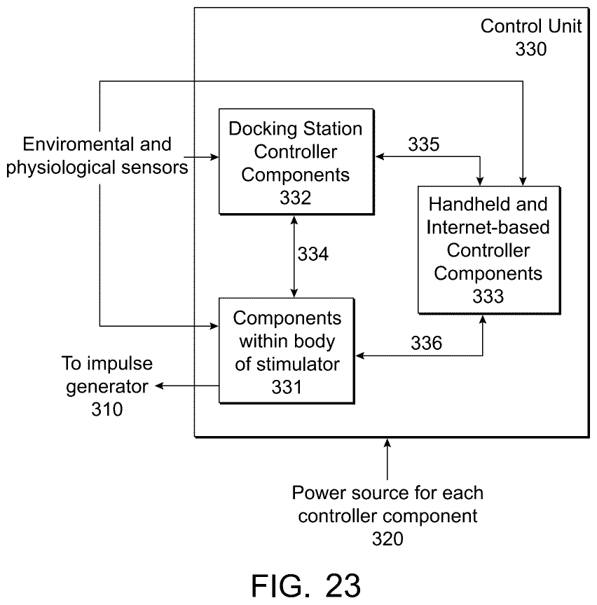

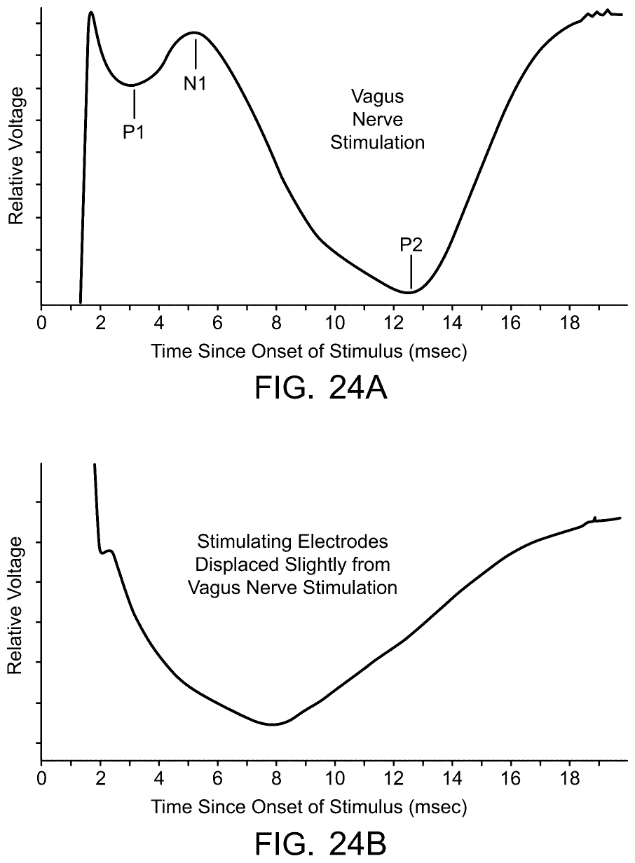

Referring now to FIG. 22, an alternative embodiment of a stimuli generation device or system 950 comprises a wearable device, such as an eye covering 952 and a controller 954 coupled to, or integrated within, the eye covering 952. Eye covering 952 may include one or more straps 956 or other coupling devices for attaching covering 952 to the user's head such that it covers the eyes. In some embodiments, controller 906 may be remote from eye covering 952 and wirelessly coupled thereto. The system may comprise one or more of integrated speakers for delivering the auditory stimuli, light transmitters, such as LEDS or the like, for delivering the visual stimuli, and sensors, such as EEG sensors, for detecting a physiological parameter of the user. Alternatively, one or more of these components may be located remote to the system and wirelessly coupled thereto. The stimulator 906 may also include an accelerometer and the associated processor or electronics for synchronizing brainwave activity with the stimuli.

In an exemplary embodiment, system 950 comprises one or more auditory stimuli generators 916, which are preferably housed within controller 954. In certain embodiments, auditory stimuli generators 916 includes speakers (not shown) configured to deliver binaural tones to one or both of the user's ears. System 950 may further comprise a motion detector 922 and one or more EEG sensors 910.

### Electronics And Software Of The Stimulator

In some embodiments, the signal waveform (FIG. 2) that is to be applied to electrodes of the stimulator is initially generated in a component of an impulse generator that is exterior to, and remote from, a mobile phone housing. The mobile phone preferably includes a software application that can be downloaded (e.g., mobile app store, USB cable, memory stick, Bluetooth connection) into the phone to receive, from the external control component, a wirelessly transmitted waveform, or to receive a waveform that is transmitted by cable, e.g., via a multi-purpose jack. If the waveforms are transmitted in compressed form, they are preferably compressed in a lossless manner, e.g., making use of FLAC (Free Lossless Audio Codec). Alternatively, the downloaded software application may itself be coded to generate a particular waveform that is to be applied to the electrodes and subsequently conveyed to the external interface of the electrode assembly. In some embodiments, the software application is not downloaded from outside the device, but is instead available internally, for example, within read-only-memory that is present within the housing of the stimulator.

In some embodiments, the waveform is first conveyed by the software application to contacts within the phone's speaker output or the earphone jack socket, as though the waveform signal were a generic audio waveform. That pseudo-audio waveform will generally be a stereo waveform, representing signals that are to be applied to the “left” and “right” electrodes. The waveform will then be conveyed to the housing of the stimulator. as follows. The housing of the stimulator may have an attached dangling audio jack that is plugged into the speaker output or the earphone jack socket whenever electrical stimulation is to be performed, or the electrical connection between the contacts of the speaker output or the earphone jack socket and the housing of the stimulator may be hard-wired. In either case, electrical circuits on a printed circuit board located under the belly of the housing of the stimulator may then shape, filter, and/or amplify the pseudo-audio signal that is received via the speaker output or earphone jack socket. An energy amplifier within the housing of the stimulator may then drive the signal onto the electrodes, in a fashion that is analogous to the use of an audio energy amplifier to drive loudspeakers. Alternatively, the signal processing and amplification may be implemented in a separate device that can be plugged into sockets on the phone and/or housing of the stimulator, to couple the software application and the electrodes.

In addition to passing the stimulation waveform from the smartphone to the stimulator housing as described herein, the smartphone may also pass control signals to the stimulator housing. Thus, the stimulation waveform may generally be regarded as a type of analog, pseudo-audio signal, but if the signal contains a signature series of pulses signifying that a digital control signal is about to be sent, logic circuitry in the stimulator housing may then be set to decode the series of digital pulses that follows the signature series of pulses, analogous to the operation of a modem.

Many of the steps that direct the waveform to the electrodes, including steps that may be controlled by the user via the touchscreen, are implemented in the above-mentioned software application. By way of example, the software application may be written for a phone that uses the Android operating system. Such applications are typically developed in the Java programming language using the Android Software Development Kit (SDK), in an integrated development environment (IDE), such as Eclipse [Mike WOLFSON. Android Developer Tools Essentials. Sebastopol, California: O'Reilly Media Inc., 2013; Ronan SCHWARZ, Phil Duston, James Steele, and Nelson To. The Android Developer's Cookbook. Building Applications with the Android SDK, Second Edition. Upper Saddle River, NJ: Addison-Wesley, 2013, the disclosure of which is incorporated herein by reference for all purposes as if copied and pasted herein; Shane CONDER and Lauren Darcey. Android Wireless Application Development, Second Edition. Upper Saddle River, NJ: Addison-Wesley, 2011; Jerome F. DIMARZIO. Android—A Programmer's Guide. New York: McGraw-Hill. 2008. pp. 1-319, the disclosure of which is incorporated herein by reference for all purposes as if copied and pasted herein]. Application programming interfaces (APIs) that are particularly relevant to the audio features of such an Android software application (e.g., MediaPlayer APIs) are described by: Android Open Source Project of the Open Handset Alliance. Media Playback, at web domain developer.android.com with subdomain/guide/topics/media/, Jul. 18, 2014, the disclosure of which is incorporated herein by reference for all purposes as if copied and pasted herein. Those APIs can be relevant to a use of the smartphone camera capabilities, as described below. Additional components of the software application are available from device manufacturers [Samsung Mobile SDK, at web domain developer.samsung.com with subdomain/samsung-mobile-sdk, Jul. 18, 2014, the disclosure of which is incorporated herein by reference for all purposes as if copied and pasted herein].

In some embodiments, the stimulator, the smartphone and/or the wearable electronic device will include a user control, such as a switch or button, that disables/enables the stimulator. Preferably, the switch will automatically disable some, many, most, or all smartphone or the wearable electronic device functions when the stimulator is enabled (and vice versa). This ensures that the medical device functionality of the smartphone or other device is completely segregated from the rest of the phone's functionality. In some embodiments, the switch will be password-controlled such that only the user/owner of the stimulator/phone will be able to enable the stimulator functionality. In one such embodiment, the switch will be controlled by a biometric scan (e.g., fingerprint, optical scan or the like) such that the stimulator functionality can only be used by the user. This ensures that only the user will be able to use the prescribed therapy in the event the phone is lost or stolen.

The stimulator and/or phone can also include software that allows the user to order more therapy doses over the internet (discussed in more detail below in connection with the docking station). The purchase of such therapy doses will require physician authorization through a prescription or the like. To that end, the software can include an authorization code for entry in order for the user to download authorization for more therapies. In some embodiments, without such authorization, the stimulator will be disabled and will not deliver therapy.

In some embodiments, the housing of the stimulator may also be joined to and/or energized by a wireless device that is not a phone (e.g., Wi-Fi enabled device, wearable electronic device, tablet). Alternatively, the stimulator may be coupled to a phone or other Wi-Fi enabled device through a wireless connection for exchanging data at short distances, such as Bluetooth or the like. In this embodiment, the stimulator housing is not attached to the smartphone and, therefore, may comprise a variety of other shapes and sizes that are convenient for the user to carry in his or her purse, wallet or pocket.

In some embodiments, the stimulator housing may be designed as part of a protective or decorative case for the phone that can be attached to the phone, similar to standard phone cases. In one such embodiment, the stimulator/case may also include additional battery life for the phone and may include an electrical connection to the phone's battery to recharge the battery (e.g., part of a Mophie® or the like). This electrical connection may also be used to couple the smartphone to the stimulator.

### Embodiments With Distributed Controllers

In some embodiments, at least some (if not all) portions of the control of the vagus nerve stimulation reside in controller components that are physically separate from the housing of the stimulator. In these embodiment, separate components of the controller and stimulator housing generally communicate with one another wirelessly, although wired or waveguide communication is possible. Suitable components include a remote computer or server, wearable computing devices, such as a smartwatch, Whoop®, Fitbit®, Garmin® or the like, a mobile phone, a mobile processing device (e.g., laptop computers or tablets) and the like. Thus, the use of wireless technology avoids the inconvenience and distance limitations of interconnecting cables.

First, the stimulator may be constructed with the minimum number of components needed to generate the stimulation pulses, with the remaining components placed in parts of the controller that reside outside the stimulator housing, resulting in a lighter and smaller stimulator housing. In fact, the stimulator housing may be made so small that it could be difficult to place, on the stimulator housing's exterior, switches and knobs that are large enough to be operated easily. Instead, the user may generally operate the device using the smartphone touchscreen.

Second, the controller 130 may be given additional functions when free from the limitation of being situated within or near the stimulator housing. For example, one may add to the controller a data logging component that records when and how stimulation has been applied to the user, for purposes of medical recordkeeping and billing. The complete electronic medical record database for the user may be located far from the stimulator (e.g., somewhere on the internet), and the billing system for the stimulation services that are provided may also be elsewhere, so it would be useful to integrate the controller into that recordkeeping and billing system, using a communication system that includes access to the internet or telephone networks.

Third, communication from the databases to the controller would also be useful for purposes of metering electrical stimulation of the user, when the stimulation is self-administered. For example, if the prescription for the user only permits only a specified amount of stimulation energy to be delivered during a single session of vagus nerve stimulation, followed by a wait-time before allowing the next stimulation, the controller can query the database and then permit the stimulation only when the prescribed wait-time has passed. Similarly, the controller can query the billing system to assure that the user's account is in order, and withhold the stimulation if there is a problem with the account.

Fourth, as a corollary of the previous considerations, the controller may be constructed to include a computer program separate from the stimulating device, in which the databases are accessed via cell phone or internet connections.

Fifth, in some applications, it may be desired that the stimulator housing and parts of the controller be physically separate. For example, when the user is a child, one wants to make it impossible for the child to control or adjust the vagus nerve stimulation. The best arrangement in that case is for the stimulator housing to have no touchscreen elements, control switches or adjustment knobs that could be activated by the child. Alternatively, any touchscreen elements, switches and knobs on the stimulator can be disabled, and control of the stimulation then resides only in a remote controller with a child-proof operation, which would be maintained under the control of a parent or healthcare provider.

Sixth, in some applications, the particular control signal that is transmitted to the stimulator by the controller will depend on physiological and environmental signals that are themselves transmitted to and analyzed by the controller. In such applications, many of the physiological and environmental signals may already be transmitted wirelessly, in which case it is most convenient to design an external part of the controller as the hub of all such wireless activity, including any wireless signals that are sent to and from the stimulator housing.

With these considerations in mind, an embodiment of can include a mobile device that may send/receive data to/from the stimulator, and may send/receive data to/from databases and other components of the system, including those that are accessible via the internet (or another network such as local area, wide area, satellite, cellular). Typically, the mobile device will be a laptop computer attached to additional components needed for it to accomplish its function. Thus, prior to any particular stimulation session, the mobile device may load into the stimulator parameters of the session, including waveform parameters, or the actual waveform.

In some embodiments, the mobile device is also used to limit the amount of stimulation energy that may be consumed by the user during the session, by charging the stimulator's rechargeable battery with only a specified amount of releasable electrical energy, which is different than setting a parameter to restrict the duration of a stimulation session. Thus, the mobile device may comprise a energy supply that may be connected to the stimulator's rechargeable battery, and the mobile device meters the recharge. As a practical matter, the stimulator may therefore use two batteries, one for applying stimulation energy to the electrodes (the charge of which may be limited by the mobile device) and the other for performing other functions. Methods for evaluating a battery's charge or releasable energy can be as disclosed in U.S. Pat. No. 7,751,891, entitled Energy supply monitoring for an implantable device, to ARMSTRONG et al, the disclosure of which is incorporated herein by reference for all purposes as if copied and pasted herein. Alternatively, some control components within the stimulator housing may monitor the amount of electrode stimulation energy that has been consumed during a stimulation session and stop the stimulation session when a limit has been reached, irrespective of the time when the limit has been reached.

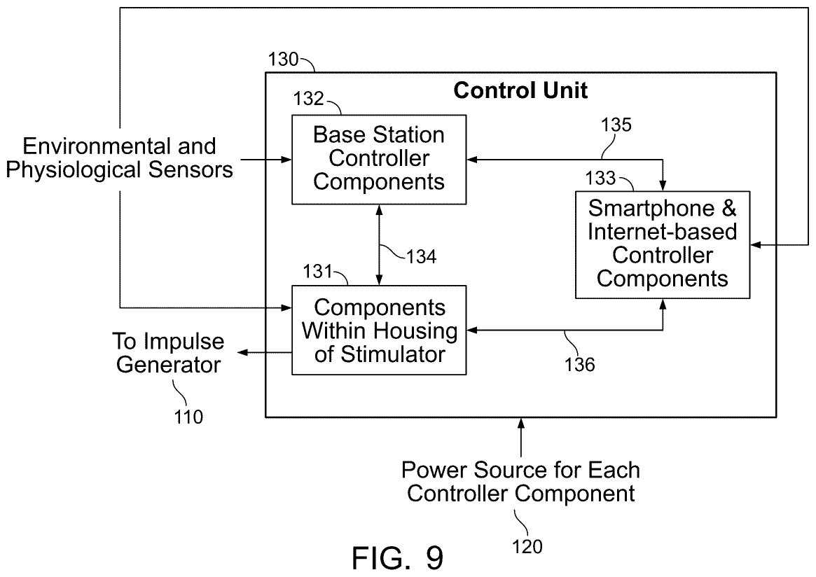

The communication connections between different components of the stimulator's controller are shown in FIG. 9, which is an expanded representation of the control unit 130 in FIG. 1. Connection between the mobile device controller components 132 and components within the stimulator housing 131 is denoted in FIG. 8 as 134. Connection between the mobile device controller components 132 and internet-based (or network based) or smartphone and/or wearable electronic components 133 is denoted as 135. Connection between the components within the stimulator housing 331 and internet-based or smartphone components 133 is denoted as 136. For example, control connections between the smartphone and stimulator housing via the audio jack socket would fall under this category, as would any wireless communication directly between the stimulator housing itself and a device situated on the internet. In principle, the connections 134, 135 and 136 in FIG. 9 may be either wired or wireless or waveguide-based. Different embodiments may lack one or more of the connections.

Although infrared or ultrasound wireless control might be used to communicate between components of the controller, they are not preferred because of line-of-sight limitations. Instead, the communication between devices preferably makes use of radio communication within unlicensed ISM frequency bands (260-470 MHz, 902-928 MHz, 2400-2.4835 GHz). Components of the radio frequency system in devices in 331, 332, and 333 typically comprise a system-on-chip transceiver with an integrated microcontroller; a crystal; associated balun & matching circuitry, and an antenna [Dag GRINI. RF Basics, RF for Non-RF Engineers. Texas Instruments, Post Office Box 655303, Dallas, Texas 75265, 2006, the disclosure of which is incorporated herein by reference for all purposes as if copied and pasted herein].

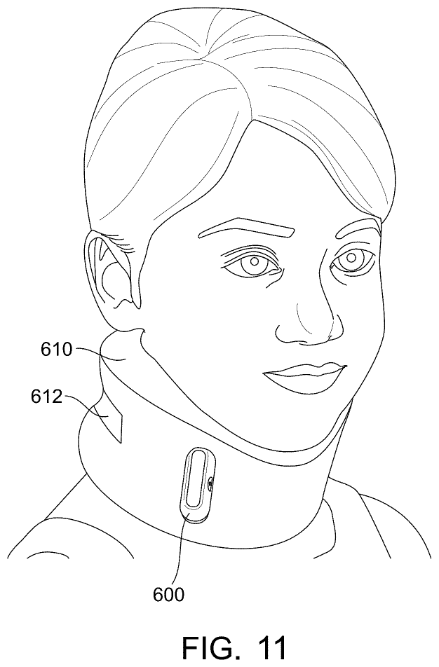

Transceivers based on 2.4 GHz offer high data rates (greater than 1 Mbps) and a smaller antenna than those operating at lower frequencies, which makes them suitable for with short-range devices. Furthermore, a 2.4 GHz wireless standard (e.g., Bluetooth, Wi-Fi, and ZigBee) may be used as the protocol for transmission between devices. Although the ZigBee wireless standard operates at 2.4 GHz in most jurisdictions worldwide, it also operates in the ISM frequencies 868 MHz in Europe, and 915 MHz in the USA and Australia. Data transmission rates vary from 20 to 250 kilobits/second with that standard. Because many commercially available health-related sensors may operate using ZigBee, its use may be recommended for applications in which the controller uses feedback and feedforward methods to adjust the user's vagus nerve stimulation based on the sensors' values, as described below in connection with FIG. 11 [ZigBee Wireless Sensor Applications for Health, Wellness and Fitness. ZigBee Alliance 2400 Camino Ramon Suite 375 San Ramon, CA 94583].

A 2.4 GHz radio has higher energy consumption than radios operating at lower frequencies, due to reduced circuit efficiencies. Furthermore, the 2.4 GHz spectrum is crowded and subject to significant interference from microwave ovens, cordless phones, 802.11b/g wireless local area networks, Bluetooth devices, etc. Sub-GHz radios enable lower energy consumption and can operate for years on a single battery. These factors, combined with lower system cost, make sub-GHz transceivers ideal for low data rate applications that need maximum range and multi-year operating life.

The antenna length needed for operating at different frequencies is 17.3 cm at 433 MHz, 8.2 cm at 915 MHz, and 3 cm at 2.4 GHz. Therefore, unless the antenna is included in a neck collar that supports the device shown in FIG. 3, the antenna length may be a disadvantage for 433 MHz transmission. The 2.4 GHz band has the advantage of enabling one device to serve in all major markets worldwide since the 2.4 GHz band is a global spectrum standard. However, 433 MHz is a viable alternative to 2.4 GHz for most of the world, and designs based on 868 and 915 MHz radios can serve the US and European markets with a single product.

Range is determined by the sensitivity of the transceiver and its output energy. A primary factor affecting radio sensitivity is the data rate. Higher data rates reduce sensitivity, leading to a need for higher output energy to achieve sufficient range. For many applications that require only a low data rate, the preferred rate is 40 Kbps where the transceiver can still use a standard off-the-shelf 20 parts per million crystal.

A signal waveform that might be transmitted wirelessly to the stimulator housing was shown in FIGS. 2B and 2C. As seen there, individual sinusoidal pulses have a period of tau, and a burst consists of N such pulses. This is followed by a period with no signal (the inter-burst period). The pattern of a burst followed by silent inter-burst period repeats itself with a period of T. For example, the sinusoidal period tau may be 200 microseconds; the number of pulses per burst may be N=5; and the whole pattern of burst followed by silent inter-burst period may have a period of T=40000 microseconds, which is comparable to 25 Hz stimulation (a much smaller value of T is shown in FIG. 2C to make the bursts discernable). When these exemplary values are used for T and tau, the waveform contains significant Fourier components at higher frequencies ( 1/200 microseconds=5000/sec). Such a signal may be easily transmitted using 40 Kbps radio transmission. Compression of the signal is also possible, by transmitting only the signal parameters tau, N, T, Emax, etc., but in that case the stimulator housing's control electronics would then have to construct the waveform from the transmitted parameters, which would add to the complexity of components of the stimulator housing.

However, because it is contemplated that sensors attached to the stimulator housing may also be transmitting information, the data transfer requirements may be substantially greater than what is required only to transmit the signal shown in FIG. 2. Therefore, the devices and methods disclosed herein may make use of any frequency band, not limited to the ISM frequency bands, as well as techniques known in the art to suppress or avoid noise and interferences in radio transmission, such as frequency hopping and direct sequence spread spectrum.

When a user is using the stimulation device to perform self-stimulation therapy, e.g., at home or at a workplace, he or she will follow the steps that are now described. It is assumed that the optimal stimulation position has already been marked on the user's neck, as described above and that a reference image of the fluorescent spots has already been acquired. The previous stimulation session will ordinarily have discharged the rechargeable batteries of the stimulator housing, and between sessions, the mobile device will have been used to recharge the stimulator at most only up to a minimum level. If the stimulator's batteries had charge remaining from the previous stimulation session, the mobile device will discharge the stimulator to a minimum level that will not support stimulation of the user.

The user can initiate the stimulation session using a wearable computing device, such as a smartwatch, Whoop® or the like, a Fitbit®, Garmin® or the like, a mobile phone, a mobile processing device (e.g., laptop computer) and the like, by invoking a computer program (on the laptop computer or through an app on the mobile phone) that is designed to initiate use of the stimulator. The programs in the smartphone and mobile device may initiate and interact with one another wirelessly, so in what follows, reference to the program (app) in the smartphone may also apply to the program in the mobile device, because both may be operating in tandem. For security reasons, the program would begin with the request for a user name and a password, and that user's demographic information and any data from previous stimulator experiences would already be associated with it in the login account. The smartphone may also be used to authenticate the user using a fingerprint or voice recognition app, or other reliable authentication methods. If the user's physician has not authorized further treatments, the mobile device will not charge the stimulator's batteries, and instead, the computer program will call or otherwise communicate with the physician's computer requesting authorization. After authorization by the physician is received, the computer program (on the laptop computer or through an app on the mobile phone) may also query a database that is ordinarily located somewhere on the internet to verify that the user's account is in order. If it is not in order, the program may then request prepayment for one or more stimulation sessions, which would be paid by the user using a credit card, debit card, PayPal, cryptocurrency, bitcoin, or the like. The computer program will also query its internal database or that of the mobile device to determine that sufficient time has elapsed between when the stimulator was last used and the present time, to verify that any required wait-time has elapsed.

Having received authorization to perform a nerve stimulation session, the user interface computer program will then ask the user questions that are relevant to the selection of parameters that the mobile device will use to make the stimulator ready for the stimulation session. The questions that the computer program asks are dependent on the condition for which the user is being treated, which for present purposes is considered to be treatment for an autoimmune disease or disorder. The questions may be things like (1) is this an acute or prophylactic treatment?(2) if acute, then how severe is your pain and in what locations, how long have you had it, (3) has anything unusual or noteworthy occurred since the last stimulation?etc.

Having received such preliminary information from the user, the computer programs will perform instrument diagnostic tests and make the stimulator ready for the stimulation session. In general, the algorithm for setting the stimulator parameters will have been decided by the physician and will include the extent to which the stimulator batteries should be charged, which the vagus nerve should be stimulated (right or left), and the time that the user should wait after the stimulation session is ended until initiation of a subsequent stimulation session. The computer will query the physician's computer to ascertain whether there have been any updates to the algorithm, and if not, will use the existing algorithm. The user will also be advised of the stimulation session parameter values by the interface computer program, so as to know what to expect.

Once the mobile device has been used to charge the stimulator's batteries to the requisite charge, the computer program (or smartphone app) will indicate to the user that the stimulator is ready for use. At that point, the user would clean the electrode surfaces, and make any other preliminary adjustments to the hardware. The stimulation parameters for the session will be displayed, and any options that the user is allowed to select may be made. Once the user is ready to begin, he or she will press a “start” button on the touchscreen and may begin the vagus nerve stimulation.

Multiple methods may be used to test whether the user is properly attempting to stimulate the vagus nerve (or another nerve or organ or muscle or bone) on the intended side of the neck (or another portion of a human body). For example, accelerometers and gyroscopes within the smartphone may be used to determine the position and orientation of the smartphone's touch screen relative to the user's expected view of the screen, and a decision by the stimulator's computer program as to which hand is being used to hold the stimulator may be made by measuring capacitance on the outside of the stimulator body, which may distinguish fingers wrapped around the device versus the ball of a thumb [Raphael WIMMER and Sebastian Boring. HandSense: discriminating different ways of grasping and holding a tangible user interface. Proceedings of the 3rd International Conference on Tangible and Embedded Interaction, pp. 359-362. ACM New York, NY, 2009, the disclosure of which is incorporated herein by reference for all purposes as if copied and pasted herein]. Pressing of the electrodes against the skin will result in a resistance drop across the electrodes, which can initiate operation of the rear camera. A fluorescent image should appear on the smartphone screen only if the device is applied to the side of the neck in the vicinity of the fluorescent spots that had been applied as a tattoo earlier. If the totality of these data indicates to the computer program that the user is attempting to stimulate the wrong vagus nerve or that the device is being held improperly, the stimulation will be withheld, and the stimulator may then communicate with the user via the interface computer program (in the mobile phone or laptop computer) to alert the user of that fact. T

Before logging off of the interface computer program, the user may also review database records and summaries about all previous treatment sessions, so as to make his or her own judgment about treatment progress. If the stimulation was part of a prophylactic treatment regimen that was prescribed by the user's physician, the user interface computer program will remind the user about the schedule for the upcoming self-treatment sessions and allow for a rescheduling if necessary.

For some users, the stimulation may be performed for as little as 60 seconds, but it may also be for up to 30 minutes or longer. The treatment is generally performed once or twice daily or several times a week, for 12 weeks or longer before a decision is made as to whether to continue the treatment. For users experiencing intermittent symptoms, the treatment may be performed only when the user is symptomatic. However, it is understood that parameters of the stimulation protocol may be varied in response to heterogeneity in the pathophysiology of users. Different stimulation parameters may also be used as the course of the user's condition changes.

### Applications Of Stimulators To The User

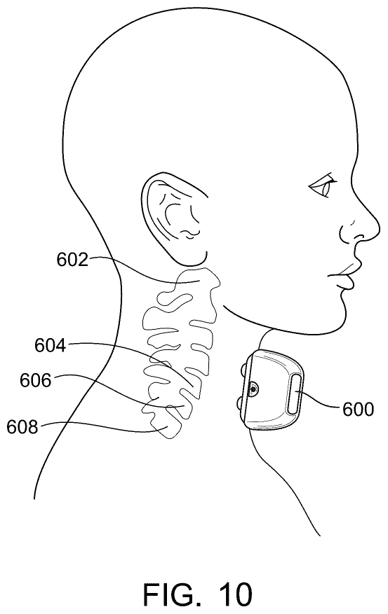

Selected nerve fibers are stimulated in different embodiments of methods that make use of the disclosed electrical stimulation devices, including stimulation of the vagus nerve at a location in the user's neck. FIG. 10 illustrates use of a stimulator 600 to stimulate the vagus nerve at that location in the neck, in which the stimulator device 600 is shown to be applied to the target location on the user's neck as described herein. For reference, FIG. 10 shows the locations of the following vertebrae: first cervical vertebra 602, the fifth cervical vertebra 604, the sixth cervical vertebra 606, and the seventh cervical vertebra 608.

Of course, it will be recognized that the vagus nerve may be stimulated through other mechanisms. For example, auricular vagal nerve stimulation involves stimulation of the auricular branch of the vagus nerve, often termed the Alderman's nerve or Arnold's nerve. This nerve may be stimulated through the transcutaneous systems and methods described herein by transmitting electrical impulses through the outer skin surface of the user's ear to the auricular branch of the vagus nerve.

FIG. 11 shows the stimulator 600 applied to the neck of a child, which is partially immobilized with a foam cervical collar 610 that is similar to ones used for neck injuries and neck pain. The collar is tightened with a strap 612, and the stimulator is inserted through a hole in the collar to reach the child's neck surface. In such applications, the stimulator may be turned on and off remotely, using a wireless controller that may be used to adjust the stimulation parameters of the controller (e.g., on/off, stimulation amplitude, frequency, etc.).

### SYSTEMS OF THE PRESENT DISCLOSURE

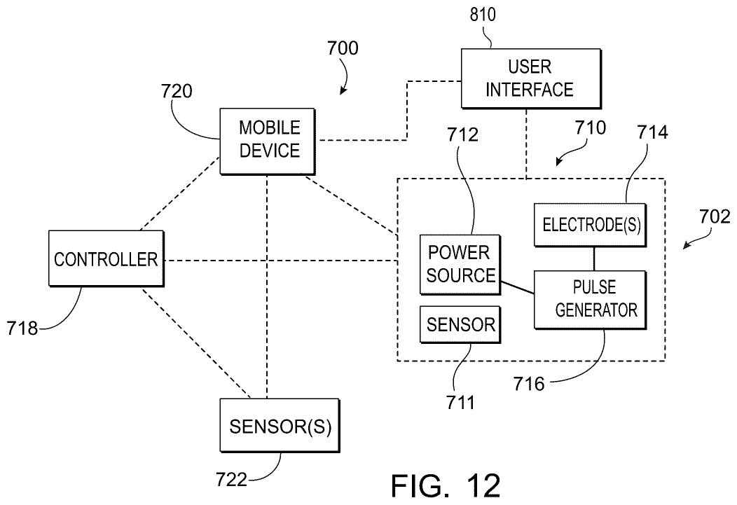

Referring now to FIG. 12, a system 700 for stimulating a nerve in a user, such as the vagus nerve, includes a stimulator 712, which may include one or more electrodes 714, a pulse generator 716 and an energy source 712. Stimulator 700 may also include one or more sensors 711, such as the position sensors described above. Electrodes 714, sensors 711, pulse generator 716 and energy source 812 may all be housed in a single housing, as described in detail above. In an alternative embodiment, electrodes 714 and/or sensors 711 are disposed separately from energy source 712 and pulse generator 716. Electrodes 714 and/or sensors 711 may be coupled to these components via wired connections or wirelessly. In the latter configuration, electrodes 714 and/or sensors 711 may include suitable electronic components coupled thereto to receive the electrical impulse(s) from pulse generator 716 and to apply those electrical impulse(s) through electrodes 714 to the user. Such electronic components may include, for example, a wireless receiver or similar component that receives the signal from a wireless transmitter coupled to pulse generator 716.

In still another embodiment, pulse generator 716 and energy source 712 are coupled to each other, either wirelessly, via wired connections, or directly in a housing that contains both components. This housing may, for example, include a wireless transmitter and may be worn by the user in manners known to those skilled in the art, so that the signal can be transmitted from the housing to electrodes 714.

System 700 further includes a controller 718 that is coupled to stimulator 702 and may be used to select or set parameters for the stimulation protocol (amplitude, frequency, pulse width, burst number, electrode positioning etc.), the treatment regimen discussed above (i.e., duration and number of doses, etc.) or alert the user as to the need to use or adjust the stimulator (i.e., an alarm). Controller 718 may be directly coupled to stimulator 702 via wired connectors or within the same housing, or it may be wirelessly coupled to stimulator 702.

Significant portions of the control of the vagus nerve stimulation may reside in controller components that are physically separate from stimulator 702. In this embodiment, separate components of the controller 718 and stimulator 702 generally communicate with one another wirelessly. Thus, the use of wireless technology avoids the inconvenience and distance limitations of interconnecting cables.

In certain embodiments, system 700 may further include one or more mobile device(s) 720 that either couple controller 718 to stimulator 702 or vice versa. Mobile device 720 may comprise a mobile phone, such as a smartphone, wearable electronic device, such as a smartwatch, iPad, laptop computer or any other mobile device having a computing function and wireless transmission technology.

In certain embodiments, system includes a suitable user interface 810 and a computer-readable storage device and/or one or more software applications that allow a user to input current user status information into controller 718. User interface 810 may be located on, for example, stimulator 702, one or more mobile devices 720 or controller 718. The mobile device(s) 720 or the stimulator 702 may include an alert or other alarm that reminds the user to input user status information on a regular time schedule. The user status information may include, for example, a current level of pain, a satisfaction level, a current mood, an amount of recent medication use (e.g., pain medication), a perceived activity level, the amount of sleep that the user has recently received or any other data related to the user's general health or recovery. This user status information is stored within controller 718 and may be displayed in a variety of different forms for the user: list form, graphical form, activity reports and the like. The user status information allows the user (and the prescribing physician) to document the user status information, and it may provide historical trends of this information (e.g., have pain levels or medication use gone down over time) to provide a more holistic picture of his/her progress with the therapy regimen.

In certain embodiments, controller 718 includes a processor that correlates the user status information with other data received from sensors 722, with the parameters of the electrical impulse and/or the overall treatment protocol (i.e., the intensity of the electrical impulse, the duration of single doses, or the number of single doses in total, or over a period of time, such as doses/day, doses/week or the like).

The physiological parameters that may include, but are not limited to, heart rate and variability, ECG, respiration depth and rate, core temperature, hydration, blood pressure, brain function, oxygenation, skin impedance, and skin temperature. Alternatively, the user status information may be correlated with certain parameters of the treatment regimen, such as the duration, amplitude and/or frequency of each single dose applied by the stimulator, the number of single doses applied by the stimulator over certain intervals of time (e.g., doses per day, per week, per month, etc.), the specific locations in which stimulation was applied (i.e., the relative position of the electrodes and the target nerve) and the like.

The processor may be configured to allow the display of this correlated information on the mobile device so that the user and/or physician can compare and track the user status information with the physiological parameters and/or the actual treatment parameters. This provides valuable data to both the user and the physician to help them visualize the effectiveness of the stimulation therapy or to allow them to modify the stimulation therapy regimen to optimize its benefits to the users. For example, the processor may determine that the therapy was more effective when the user applied the single doses for a longer duration or at a higher amplitude. In another example, the processor may determine that the therapy was more effective when the user included certain intervals between doses (e.g., 4-6 hours), or applied a certain number of doses per day or week. In yet another example, the processor may determine that the therapy was more effective when the relative positions of the electrodes and the target nerve were within a certain range. In this latter embodiment, the data could be used to demonstrate that the user received more effective therapy if the electrodes were positioned close enough to the target nerve to achieve effective stimulation thereof. In addition, the processor could pinpoint the distance between the electrodes and the target nerve wherein the stimulation becomes less effective.

In addition, this provides a historical record of this effectiveness so that the user does not have to remember the user status information at, for example, follow-up visits with the physician. For example, if the user sees that certain dosing levels and/or electrode positions of the device (and/or dosing levels that substantially track the prescribing physician's recommendations) correlate with lower pain levels, higher satisfaction, better moods, etc., the user will understand that compliance with the therapy regimen (e.g., routine, timing and duration) provides better outcomes. This understanding may provide better user compliance with the therapy regimen.

One or more of the mobile device(s) 702 preferably includes one or more software applications that display information that enhances the user experience with stimulator 702 and enables the user to track the progress he/she has made with the therapy regimen. For example, upon opening the application and creating a profile, the user may be prompted to provide baseline information on user status, such as mood, pain-level, prescribed medications and the like. The software application may also be configured to prompt the user to set goals or milestones for his/her treatment, such as pain-free activities. The software application may provide a dashboard or similar display that provides a summary of the data that has been collected during the therapy regimen. This summary data may include, for example, progress towards milestones or goals achieved, progress on recovery, such as pain levels, emotional state and/or activity levels and the like. This information may help the user avoid recovery setbacks and improve compliance with the therapy regimen.

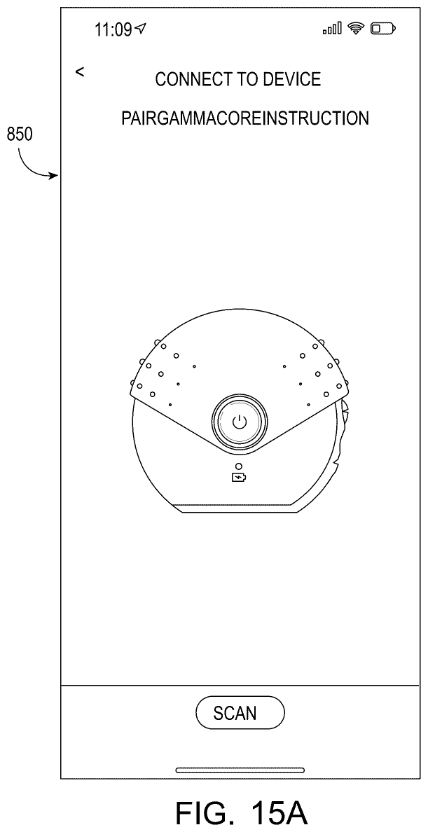

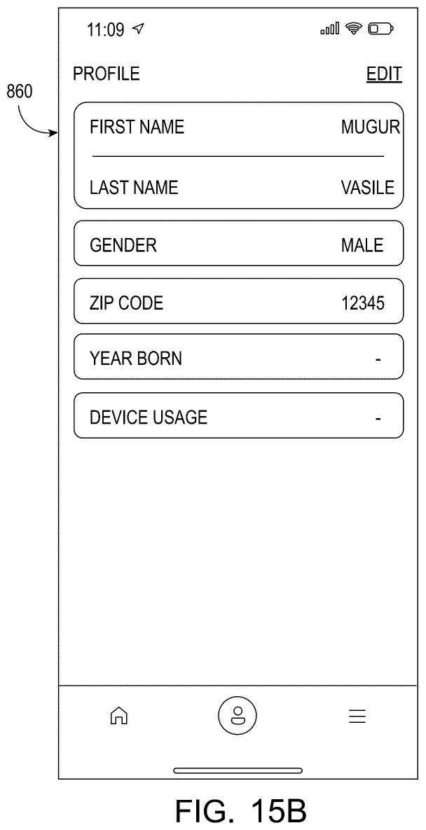

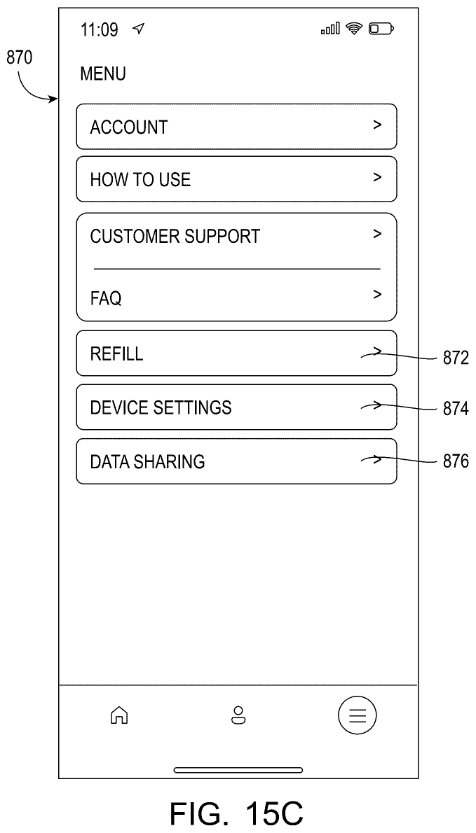

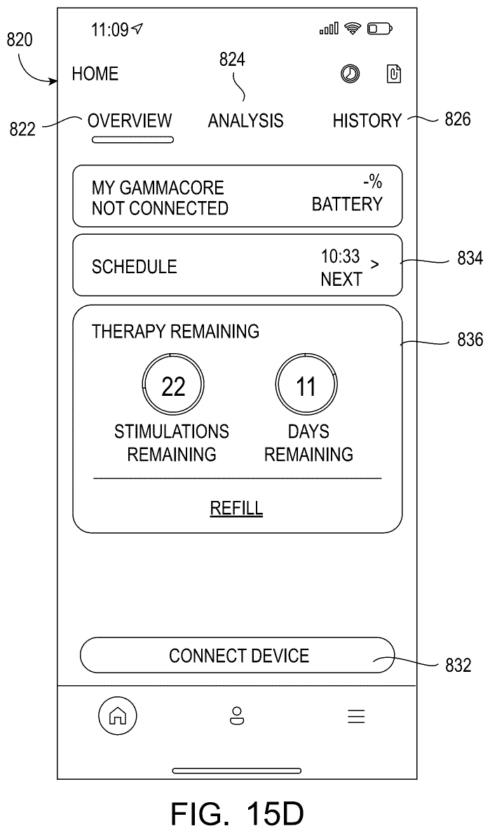

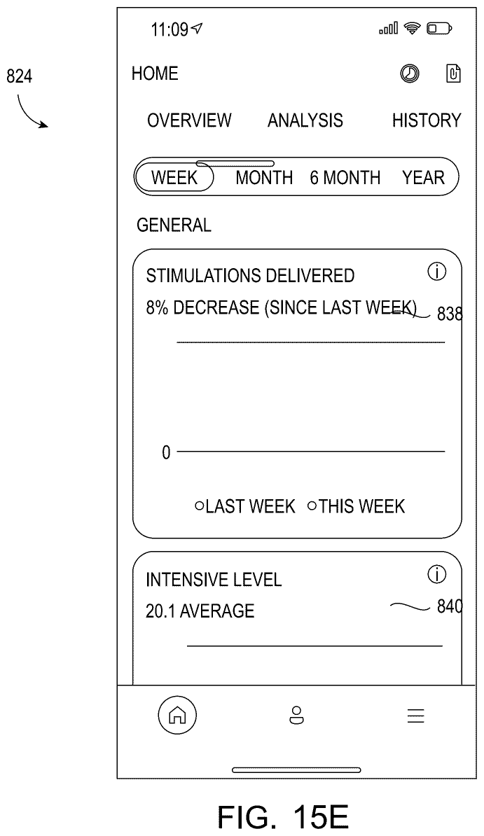

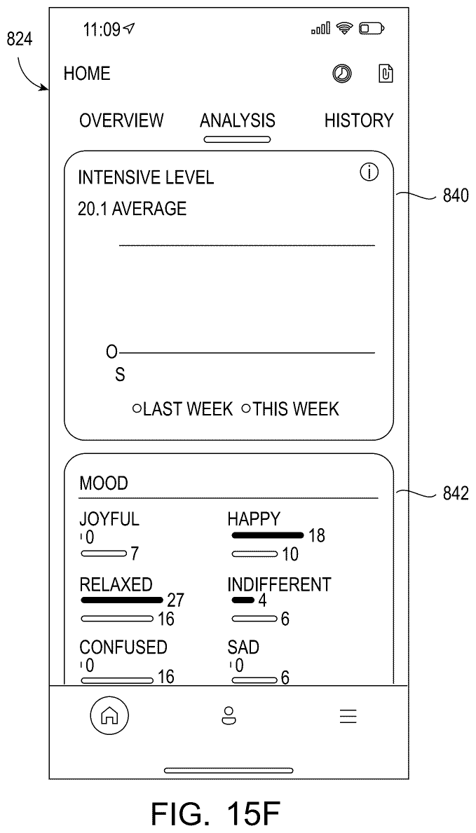

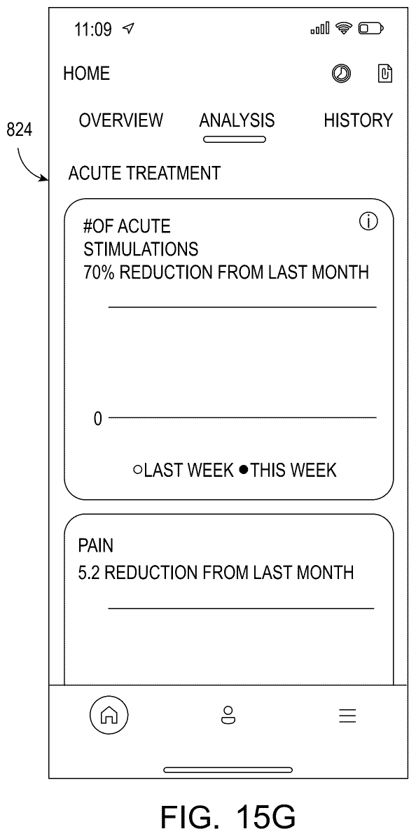

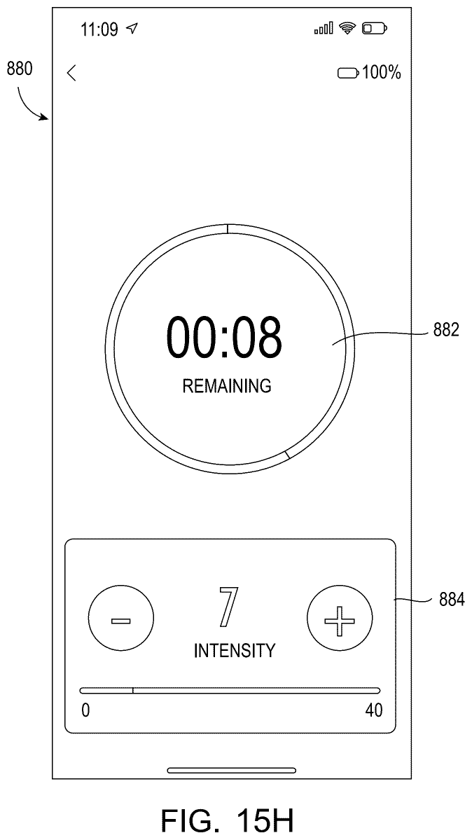

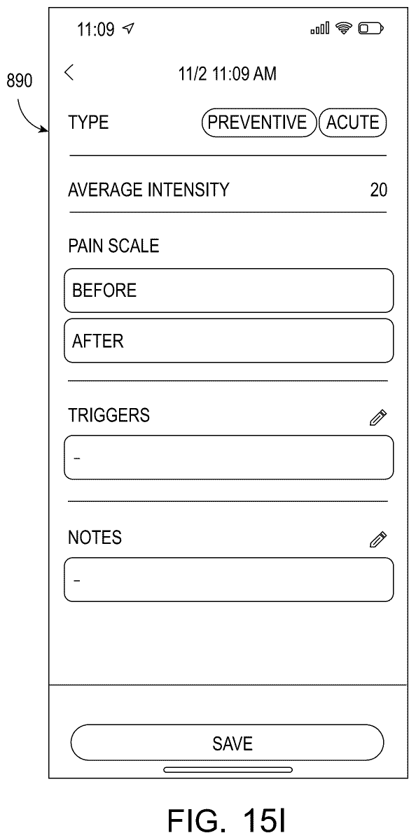

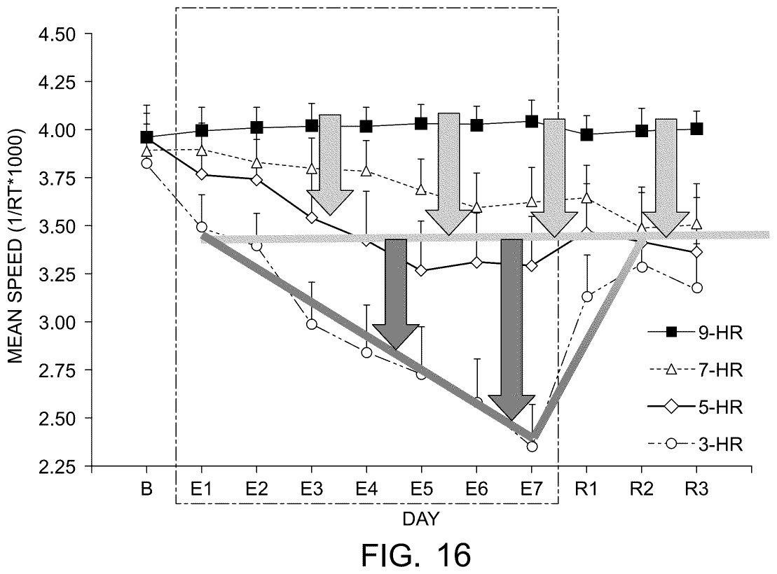

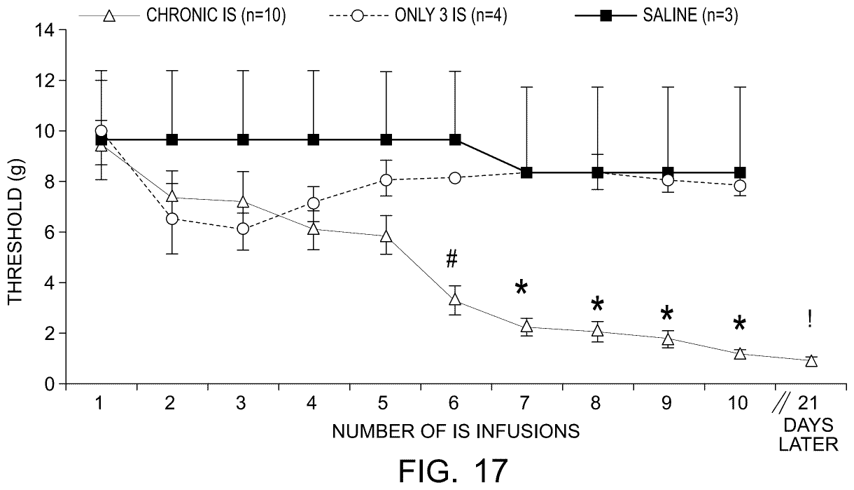

FIGS. 15A-15I illustrates one embodiment of a user interface 810 for use with system 700. Referring now to FIG. 15A, when the user opens up the software application, there is a screen or page 850 that provides instructions for wirelessly pairing the stimulator with the application. These instructions may include a prompt to input an authorization code that allows the stimulator to pair with the application. This authorization code may be the same, or different, from the authorization code that the patient submits in order to fill or refill the stimulator with a certain number of authorized or prescribed doses for treatment.

Referring now to FIG. 15B, a profile screen 860 may include a variety of personal information inputted by the user, such as name, gender, address, age, or the disorder or disease being treated. The profile screen 860 may also include a user status page (not shown) that includes various prompts for the user to enter certain status information at the beginning, during or between stimulation sessions. As discussed above, the status information may include general status information, such as mood, level of pain, or the like, or more detailed status information specifically related to the patient's disorder or disease. In one such example, the stimulator may be used for treatment of addiction, such as opioid use disorder or others. In this example, the user status information may include, any drugs taken prior to, or during, the time period of the treatment, desires or cravings to take drugs, withdrawal symptoms, tolerance and the like. In another example, the stimulator may be used to treat headache, such as migraine, cluster headache, tension headache, PTSD or the like. In this example, the user status information may include pain level or the location of the pain (i.e., one side of head, diffuse, etc.), light, noise or smell sensitivities, nausea, vomiting, loss of appetite, fatigue, dizziness or blurred vision and the like. This status information is stored within the application and may be correlated with the treatment parameters.

Referring now to FIG. 15C, the software application may include an account page 870 that allows the user to view his account (i.e., payments made for prescriptions and the like), view pages that provide information on how to operate the device, contact customer support through the software application or other methods (phone numbers, emails, etc.), or review FAQs that are provided in the software application. The account page 870 may also include a refill page 872 that allows the patient to refill the stimulator, i.e., increase the number of single doses and/or duration of time that the stimulator is authorized to apply to the user. This refill page 872 may be linked, for example, to a caregiver's processing device such that the caregiver can provide authorization for such refill. The refill page 872 may also be linked to a payment screen that allows the patient to pay for the refill.

Account page 870 may also include a device settings page 874 that provides information on the current device settings (intensity, duration, waveform, frequency, etc.). Alternatively, or in addition, the device setting pages 874 may allow the user to adjust the device settings from the mobile application (rather than directly on the device). This provides a more convenient method for the patient to adjust settings. For example, the patient may adjust the settings prior to placing the device against his/her skin surface so that the patient only needs to hold the device against the skin to apply the stimulation therapy (rather than also adjusting settings at the same time). Alternatively, the patient may find it easier to adjust the settings during stimulation from the application than on the device itself.

Account page 870 may also include a data sharing screen 876 that allows the patient to share certain data in the application with, for example, a caregiver. In some embodiments, this data will automatically be shared with the caregiver. In other embodiments, the patient may select the type of data that is shared.

Referring now to FIG. 15H, the software application may include a stimulation screen 880 that provides information during a stimulation session and/or allows the patient to adjust a parameter of stimulation during the session. For example, stimulation screen 880 may include a time counter 882 that tracks the duration of the session and provides a time countdown for the user to visualize how much time is left in the stimulation session. Stimulation screen 880 may also include an intensity display 884 that indicates the current intensity level of the stimulation session and/or allows the patient to adjust the intensity during the stimulation session.

Referring now to FIG. 15D, a home screen 820 may include a variety of data and information for the user. In this embodiment, home screen 820 includes an overview page 822, an analysis page 824 and a history page 826. Overview page 822 includes a device connection indicator 832, a schedule 834 for when the user should apply the next dose of electrical stimulation, and a schedule 836 of how many doses and/or how much time are left on the patient's prescription. The application may also include alerts that can be turned ON to remind the patient that it is time for a dose according to the treatment regimen prescribed by the physician and/or agreed to by the patient. The physician may also have the ability edit this reminder feature in the event that the treatment regimen is changed.

The stimulator 702 may be configured to deactivate and automatically turn OFF and not deliver any further doses when either the number of doses or number of days left reaches zero. In one embodiment, the stimulator 702 requires a new authorization code in order to turn back ON once it has been deactivated. A more complete description of this authorization code can be found in co-pending, commonly assigned U.S. patent application Ser. No. 16/229,299, filed Dec. 21, 2018 and U.S. Pat. No. 17,002,347, filed Aug. 25, 2022, the complete disclosures of which are incorporated herein by reference in their entirety for all purposes.

Referring now to FIG. 15E, analysis page 824 may provide a variety of data and information that allows the user to track usage of the device and/or compare the usage of the device over time. For example, analysis page 824 provides information related to the change in the number of stimulations applied by the device over a time period, such as a week, month, six months, or a year (838). These stimulations may be broken up into single doses, multiple doses/day, or the like. Analysis page 824 also provides information related to the average, maximum or minimum intensity or amplitude of the electrical signal applied over a time period, such as a week, month, six months, or a year (840).

In certain embodiments, analysis page 824 may provide additional information, such as a comparison between user status information inputted by the user and/or physiological parameters measured by one of the sensors with the actual treatment parameters (see FIG. 15G). This may, for example, indicate that a decrease in number of stimulations over a weekly period resulted in a reduced in treatment effectiveness, which may be evidenced by changes in user status information (e.g., more pain, increased pain medication, change in mood or the like) and/or changes in physiological parameters, such as those parameters associated with the symptoms of the disorder or disease being treated.

FIG. 15F illustrates one example wherein the patient has inputted status information 842 related to their mood at certain time points. This data can be broken up into certain categories, e.g., joyful, happy, relaxed, indifferent, etc. These categories may be automatically included in the software application, or the application may allow the user to input their own categories. With each category, the patient may be able to rank his/her mood with respect to that category on a scale, or 1-5, 1-10, 1-20, 1-100 or the like. The application may be further configured to average the user's rankings of moods over a period of time. This information can then be compared to the actual parameters of stimulation and/or the treatment paradigm that has been applied by the patient over that time period.

For example, analysis page 824 may indicate that the intensity level of the stimulation has decreased over a time period. This reduction in intensity may be, for example, correlated with a change in mood or another user status data point (e.g., less joyful, more pain, etc.). In this instance, the software application may be configured to automatically adjust the intensity level of the stimulation and/or simply recommend to the patient that he/she increase the intensity level by a certain amount.

In another example, analysis page 824 may indicate that the number of single doses of stimulation or the duration of each single dose has decreased over a time period. This reduction in duration or number of doses may be, for example, correlated with a change in mood or another user status data point (e.g., less joyful, more pain, etc.). In this instance, the software application may be configured to automatically adjust the intensity level of the stimulation and/or simply recommend to the patient that he/she increase the intensity level by a certain amount.

Referring now to FIG. 15I, the software application may include a treatment regimen page 890 that provides separate pages depending on the type of treatment, e.g., acute, chronic or preventative. These pages may contain a variety of useful information related to the history of the device parameters applied for each treatment regimen, e.g., average intensity, average duration, cumulative number of single doses applied, number of single doses applied over a particular time period and the like. These pages may also provide screens or prompts that allow the user to input status information, such as pain on a scale before and after stimulation. As discussed above, this status information may be correlated with the actual device parameters used to provide the patient or caregiver with valuable information as to the treatment effectiveness.

Stimulator 702 may also transmit other information to mobile device (s) 720, controller 718 or directly to a separate processing device (e.g., one operated by a caregiver). This information may include, for example, error data and/or incomplete circuit data produced by stimulator 702. For example, if the stimulator 702 produces an incomplete circuit data, this could mean that the patient requires assistance in placement of the electrodes. If the stimulator 702 produces error data, this could mean that the patient requires assistance troubleshooting stimulator 702.

In certain applications, system 700 may include a patient or user software application and a separate caregiver (e.g., physician) software application. In an exemplary embodiment, the physician software application may be configured to allow the data from individual patients to be aggregated together to form data across a plurality of different patients. This aggregated data may allow the physician to determine the overall effectiveness of the therapy across multiple patients. In addition, it may allow the physician to better understand the impact of usage of the device with the effectiveness of the therapy. For example, the data may show that increased usage of the device and/or improved compliance with the therapy regimen increases overall effectiveness or reduction in pain.

In certain embodiments, the physician software application may be configured to automatically produce reports of complied data from system 700 and/or stimulator 702 that may include, for example, patient compliance with the therapy regimen, patient status data (e.g., pain), physiological parameters and/or the actual treatment parameters. The software application may be designed to aggregate these data into single reports that allow the physician to easily compare, for example, treatment parameters with pain, patient satisfaction, medication user, activity levels and the like.

System 700 may further include a recharging outlet or station (also not shown) configured to receive a rechargeable battery. Alternatively, the battery may comprise an outlet or other coupling element for directly charging the battery with a suitable electrical connector (i.e., without removing the battery from the stimulator housing). Providing a rechargeable battery that may be easily switched out allows 24 hour use of the device, which may increase the effectiveness of the device. In other embodiments, the energy source may be located exterior to the housing and either directly connected thereto with wires or other electrical connections, or wireless coupled to the housing via a suitable wireless energy transmitter/receiver device.

In certain embodiments, the energy source includes a data storage component (not shown) coupled to a processor within stimulation device 710. The processor is configured to transfer data, such as motion data, usage levels, or any other data collected by the processor, to the data storage component. The data storage component may be accessed by a separate processor external to the stimulation device (e.g., in the mobile device or a separate processing device) when the battery is removed for recharging. This allows large amounts of data to be transferred from the stimulation device to the mobile device, i.e., larger amounts of data that may be possible through wireless transmission alone.

In addition to position sensors 711, system 700 may further include one or more additional sensors (not shown) used for detecting certain physiological parameters of the patient based on the stimulation of the nerve. The preferred sensors will include ones ordinarily used for ambulatory monitoring. For example, the sensors may comprise those used in conventional Holter and bedside monitoring applications, for monitoring heart rate and variability, ECG, respiration depth and rate, core temperature, hydration, blood pressure, brain function, oxygenation, skin impedance, and skin temperature. The sensors may be embedded in garments or placed in sports wristwatches, as currently used in programs that monitor the physiological status of soldiers [G. A. SHAW, A. M. Siegel, G. Zogbi, and T. P. Opar. Warfighter physiological and environmental monitoring: a study for the U.S. Army Research Institute in Environmental Medicine and the Soldier Systems Center. MIT Lincoln Laboratory, Lexington MA. 1 Nov. 2004, pp. 1-141]. The ECG sensors should be adapted to the automatic extraction and analysis of particular features of the ECG, for example, indices of P-wave morphology, as well as heart rate variability indices of parasympathetic and sympathetic tone. Measurement of respiration using noninvasive inductive plethysmography, mercury in silastic strain gauges or impedance pneumography is particularly advised, in order to account for the effects of respiration on the heart. A noninvasive accelerometer may also be included among the ambulatory sensors, in order to identify motion artifacts. An event marker may also be included in order for the patient to mark relevant circumstances and sensations.

For brain monitoring, the sensors may comprise ambulatory EEG sensors [CASSON A, Yates D, Smith S, Duncan J, Rodriguez-Villegas E. Wearable electroencephalography. What is it, why is it needed, and what does it entail?IEEE Eng Med Biol Mag. 29(3,2010):44-56] or optical topography systems for mapping prefrontal cortex activation [Atsumori H, Kiguchi M, Obata A, Sato H, Katura T, Funane T, Maki A. Development of wearable optical topography system for mapping the prefrontal cortex activation. Rev Sci Instrum. 2009 April; 80(4):043704]. Signal processing methods, comprising not only the application of conventional linear filters to the raw EEG data, but also the nearly real-time extraction of non-linear signal features from the data, may be considered to be a part of the EEG monitoring [D. Puthankattil SUBHA, Paul K. Joseph, Rajendra Acharya U, and Choo Min Lim. EEG signal analysis: A survey. J Med Syst 34(2010):195-212]. In the present application, the features would include EEG bands (e.g., delta, theta, alpha, beta).

For any given position of the stimulator relative to the vagus nerve, it is also possible to infer the amplitude of the electric field that it produces in the vicinity of the vagus nerve. This is done by calculation or by measuring the electric field that is produced by the stimulator as a function of depth and position within a phantom that simulates the relevant bodily tissue [Francis Marion MOORE. Electrical Stimulation for pain suppression: mathematical and physical models. Thesis, School of Engineering, Cornell University, 2007; Bartosz SAWICKI, Robert Szmurlo, Przemyslaw Plonecki, Jacek Starzynski, Stanislaw Wincenciak, Andrzej Rysz. Mathematical Modelling of Vagus Nerve Stimulation. pp. 92-97 in: Krawczyk, A. Electromagnetic Field, Health and Environment: Proceedings of EHE'07. Amsterdam, IOS Press, 2008]. Thus, in order to compensate for movement, the controller may increase or decrease the amplitude of the output from the stimulator (u) in proportion to the inferred deviation of the amplitude of the electric field in the vicinity of the vagus nerve, relative to its desired value.

Various corresponding structures, materials, acts, and equivalents of all means or step plus function elements in various claims below are intended to include any structure, material, or act for performing the function in combination with other claimed elements as specifically claimed. Various embodiments were chosen and described in order to best explain various principles of this disclosure and various practical applications thereof, and to enable others of ordinary skill in a pertinent art to understand this disclosure for various embodiments with various modifications as are suited to a particular use contemplated.

Various diagrams depicted herein are illustrative. There can be many variations to such diagrams or steps (or operations) described therein without departing from various spirits of this disclosure. For instance, various steps can be performed in a differing order or steps can be added, deleted or modified. All of these variations are considered a part of this disclosure. People skilled in an art to which this disclosure relates, both now and in future, can make various improvements and enhancements which fall within various scopes of various claims which follow.

For example, it will also be appreciated that some the devices and methods can be applied to other tissues and nerves of the body, including but not limited to other parasympathetic nerves, sympathetic nerves, spinal or cranial nerves, muscles, peripheral nerve stimulation, spinal cord stimulation, neuromuscular electrical stimulation (NMES) or other nerves, such as abdominal aortic plexus, abducens nerve, accessory nerve, accessory obturator nerve, alderman's nerve, anococcygeal nerve, ansa cervicalis, anterior interosseous nerve, anterior superior alveolar nerve, auerbach's plexus, auriculotemporal nerve, axillary nerve, brachial plexus, buccal branch of the facial nerve, buccal nerve, cardiac plexus, cavernous nerves, cavernous plexus, celiac ganglia, cervical branch of the facial nerve, cervical plexus, chorda tympani, ciliary ganglion, coccygeal nerve, cochlear nerve, common fibular nerve, common palmar digital nerves of median nerve, deep branch of the radial nerve, deep fibular nerve, deep petrosal nerve, deep temporal nerves, diagonal band of broca, digastric branch of facial nerve, dorsal branch of ulnar nerve, dorsal nerve of clitoris, dorsal nerve of the penis, dorsal scapular nerve, esophageal plexus, ethmoidal nerves, external laryngeal nerve, external nasal nerve, facial nerve, femoral nerve, frontal nerve, gastric plexuses, geniculate ganglion, genital branch of genitofemoral nerve, genitofemoral nerve, glossopharyngeal nerve, greater auricular nerve, greater occipital nerve, greater petrosal nerve, hepatic plexus, hypoglossal nerve, iliohypogastric nerve, ilioinguinal nerve, inferior alveolar nerve, inferior anal nerves, inferior cardiac nerve, inferior cervical ganglion, inferior gluteal nerve, inferior hypogastric plexus, inferior mesenteric plexus, inferior palpebral nerve, infraorbital nerve, infraorbital plexus, infratrochlear nerve, intercostal nerves, intercostobrachial nerve, intermediate cutaneous nerve, internal carotid plexus, internal laryngeal nerve, interneuron, jugular ganglion, lacrimal nerve, lateral cord, lateral cutaneous nerve of forearm, lateral cutaneous nerve of thigh, lateral pectoral nerve, lateral plantar nerve, lateral pterygoid nerve, lesser occipital nerve, lingual nerve, long ciliary nerves, long root of the ciliary ganglion, long thoracic nerve, lower subscapular nerve, lumbar nerves, lumbar plexus, lumbar splanchnic nerves, lumboinguinal nerve, lumbosacral plexus, lumbosacral trunk, mandibular nerve, marginal mandibular branch of facial nerve, masseteric nerve, maxillary nerve, medial cord, medial cutaneous nerve of arm, medial cutaneous nerve of forearm, medial cutaneous nerve, medial pectoral nerve, medial plantar nerve, medial pterygoid nerve, median nerve, meissner's plexus, mental nerve, middle cardiac nerve, middle cervical ganglion, middle meningeal nerve, motor nerve, muscular branches of the radial nerve, musculocutaneous nerve, mylohyoid nerve, nasociliary nerve, nasopalatine nerve, nerve of pterygoid canal, nerve to obturator internus, nerve to quadratus femoris, nerve to the piriformis, nerve to the stapedius, nerve to the subclavius, nervus intermedius, nervus spinosus, nodose ganglion, obturator nerve, oculomotor nerve, olfactory nerve, ophthalmic nerve, optic nerve, otic ganglion, ovarian plexus, palatine nerves, palmar branch of the median nerve, palmar branch of ulnar nerve, pancreatic plexus, patellar plexus, pelvic splanchnic nerves, perforating cutaneous nerve, perineal branches of posterior femoral cutaneous nerve, perineal nerve, petrous ganglion, pharyngeal branch of vagus nerve, pharyngeal branches of glossopharyngeal nerve, pharyngeal nerve, pharyngeal plexus, phrenic nerve, phrenic plexus, posterior auricular nerve, posterior branch of spinal nerve, posterior cord, posterior cutaneous nerve of arm, posterior cutaneous nerve of forearm, posterior cutaneous nerve of thigh, posterior scrotal nerves, posterior superior alveolar nerve, proper palmar digital nerves of median nerve, prostatic plexus (nervous), pterygopalatine ganglion, pudendal nerve, pudendal plexus, pulmonary branches of vagus nerve, radial nerve, recurrent laryngeal nerve, renal plexus, sacral plexus, sacral splanchnic nerves, saphenous nerve, sciatic nerve, semilunar ganglion, sensory nerve, short ciliary nerves, sphenopalatine nerves, splenic plexus, stylohyoid branch of facial nerve, subcostal nerve, submandibular ganglion, suboccipital nerve, superficial branch of the radial nerve, superficial fibular nerve, superior cardiac nerve, superior cervical ganglion, superior ganglion of glossopharyngeal nerve, superior ganglion of vagus nerve, superior gluteal nerve, superior hypogastric plexus, superior labial nerve, superior laryngeal nerve, superior lateral cutaneous nerve of arm, superior mesenteric plexus, superior rectal plexus, supraclavicular nerves, supraorbital nerve, suprarenal plexus, suprascapular nerve, supratrochlear nerve, sural nerve, sympathetic trunk, temporal branches of the facial nerve, third occipital nerve, thoracic aortic plexus, thoracic splanchnic nerves, thoraco-abdominal nerves, thoracodorsal nerve, tibial nerve, transverse cervical nerve, trigeminal nerve, trochlear nerve, tympanic nerve, ulnar nerve, upper subscapular nerve, uterovaginal plexus, vagus nerve, ventral ramus, vesical nervous plexus, vestibular nerve, vestibulocochlear nerve, zygomatic branches of facial nerve, zygomatic nerve, zygomaticofacial nerve, or zygomaticotemporal nerve.

1. A system for enhancing sleep, the system comprising:
a nerve stimulator comprising an electrode configured for contacting the outer skin surface at, or near a target location;
an energy source coupled to the stimulator, wherein the energy source is configured to generate at least one electrical impulse and to transmit the at least one electrical impulse transcutaneously from the electrode through the outer skin surface of the user to a selected nerve in the user adjacent to, or near, the target location; and
a sensory stimulator configured to deliver one or more stimuli to a sense organ or a brain of a user.

2. The system of claim 1, wherein the sensory stimulator comprises one or more of a visual stimulator, an auditory stimulator, an olfactory stimulator, a tactile stimulator or a combination thereof.

3. The system of claim 1, wherein the sensory stimulator comprises an auditory stimulator and a visual stimulator.

4. The system of claim 3, wherein the auditory stimulator is configured to deliver binaural tones to the brain of the user.

5. The system of claim 4, further comprising a wearable device comprising one or more sensors for measuring brainwave activity and a processor configured to compute an effective brainwave frequency based on the brainwave activity.

6. The system of claim 5, wherein the binaural tones are synchronized with the effective brainwave frequency.

7. The system of claim 4, wherein the visual stimulator is configured to deliver light patterns synchronized with the binaural tones.

8. The system of claim 7, wherein the processor is configured to adjust the binaural tones and the light patterns to entrain the brain of the user to slower frequencies for deep sleep.

9. The system of claim 8, wherein the binaural tones have frequencies of about 10 Hz to about 100 Hz.

10. The system of claim 9, wherein the binaural tones have a beat frequency of about 0.5 to about 4 Hz.

11. The system of claim 7, wherein the light patterns comprise light waves have a wavelength of about 600 nm to about 720 nm.

12. The system of claim 1, wherein the nerve stimulator comprises a housing, wherein the electrode is coupled to the housing.

13. The system of claim 9, wherein the energy source is disposed within the housing.

14. The system of claim 9, further comprising a signal generator disposed within the housing and electrically coupled to the energy source and the electrode.

15. The system of claim 1, wherein the electrical impulse comprises pulses having a frequency of about 1 kHz to about 20 kHz.

16. The system of claim 1, wherein the electrical impulse comprises bursts of pulses, with each burst having a frequency of about 1 to about 100 bursts per second and each pulse has a duration of about 50 to about 1000 microseconds in duration.

17. The system of claim 14, wherein the bursts each comprise about 2 to 20 pulses and the bursts are separated by an inter-burst period that comprises zero pulses.

18. The system of claim 1, wherein the nerve is a vagus nerve.

19. The system of claim 1, wherein the electrode is configured for contacting an outer skin surface of the neck of the user.

20. The system of claim 1, wherein the energy source is configured to transmit a plurality of electrical impulses to the selected nerve according to a treatment paradigm.

21. The system of claim 20, wherein the treatment paradigm is sufficient to reduce inflammation in the brain of the user.

22. The system of claim 20, wherein the treatment paradigm is sufficient to alter a microglia in a central nervous system of the user from a substantially pro-inflammatory state to a substantially non-inflammatory state.

23. The system of claim 22, wherein the treatment paradigm is sufficient to reduce astrocytic activation with the central nervous system of the user.

24. The system of claim 20, wherein the treatment paradigm is sufficient to increase glymphatic clearance of waste products within the brain of the user.

25. The system of claim 24, wherein the waste products comprise beta-amyloid, tau proteins and oxidative byproducts.

26. A method for enhancing sleep, the method comprising:
transmitting at least one electrical impulse transcutaneously from an electrode through the outer skin surface of the user to a selected nerve in the user adjacent to, or near, the target location; and
delivering one or more sensory stimuli to a sense organ or a brain of the user.

27. The method of claim 25, wherein the electrical impulse is transmitted to the selected nerve before the user is asleep.

28. The method of claim 25, wherein the sensory stimuli is delivered to the brain of the user while the user is asleep.

29. The method of claim 25, further comprising delivering a plurality of the electrical impulses to the selected nerve according to a treatment paradigm.

30. The method of claim 28, wherein the treatment paradigm is sufficient to reduce inflammation in the brain of the user.

31. The method of claim 28, wherein the treatment paradigm is sufficient to alter a microglia in a central nervous system of the user from a substantially pro-inflammatory state to a substantially non-inflammatory state.

32. The method of claim 30, wherein the treatment paradigm is sufficient to reduce astrocytic activation with the central nervous system of the user.

33. The method of claim 28, wherein the treatment paradigm is sufficient to increase glymphatic clearance of waste products within the brain of the user.

34. The method of claim 32, wherein the waste products comprise beta-amyloid, tau proteins and oxidative byproducts.

35. The method of claim 26, wherein the sensory stimuli comprises one or more of a visual stimuli, an auditory stimuli, an olfactory stimuli, a tactile stimuli or a combination thereof.

36. The method of claim 35, wherein the sensory stimuli comprises a visual stimuli and an auditory stimuli.

37. The method of claim 36, further comprising delivering binaural tones to the brain of the user.

38. The method of claim 37, further comprising measuring brainwave activity and determining an effective brainwave frequency.

39. The method of claim 38, further comprising synchronizing the binaural tones with the effective brainwave frequency.

40. The method of claim 39, further comprising delivering light patterns synchronized with the binaural tones.

41. The method of claim 40, further comprising adjusting the binaural tones and the light patterns to entrain the brain of the user to slower frequencies of slow wave sleep.

42. The method of claim 41, wherein the binaural tones have frequencies of about 10 Hz to about 100 Hz.

43. The method of claim 41, wherein the binaural tones have a beat frequency of about 0.1 to about 1 Hz.

44. The method of claim 41, wherein the light patterns comprise light waves have a wavelength of about 600 nm to about 650 nm.

45. The method of claim 26, wherein the electrical impulse comprises pulses having a frequency of about 1 kHz to about 20 kHz.

46. The method of claim 26, wherein the electrical impulse comprises bursts of pulses, with each burst having a frequency of about 1 to about 100 bursts per second and each pulse has a duration of about 50 to about 1000 microseconds in duration.

47. The method of claim 46, wherein the bursts each comprise about 2 to 20 pulses and the bursts are separated by an inter-burst period that comprises zero pulses.

48. The method of claim 26, wherein the selected nerve is a vagus nerve.
